SEQUENCE LISTING (30 sequences)

SEQ ID NO: 1 - protein, 361 aa, Triticum aestivum
  VARIANT (112)..(112): Glu
  VARIANT (241)..(241): Val
MANQDVVLRPDMGGIAAAAAMPGSSSRAIFACRGAASSSLRRRSAVDDEFRAGSPCATSWVVQAMRASSPARSAAVDEYAAWTRKHPSALASFEQIAAAAKGKQVVVFLDYDGTLSPIVADPDTAVISGEMREAVRGVAKHFPAAIVTGRCVEKVCSFVGLSELYYAGSHGMDIKGPGSNAEEVLLQPAREFLPVIAEVYEALVEKTKSTPGARVENNKFCLSVHFRCVDEKRWSPLAEQAKEVLRDYPDLSLNEGRKVLEIRPSIMWDKGKAVEFLLQSLGFDGRSDVLPLYLGDDRTDEDAFKMLRKRGHGLGILVSKCPRETDASYSLQDPTEVMEFLHRLVQWRRRRSSSSAMRARV

SEQ ID NO: 2 - DNA, 1086 bp, Artificial Sequence
  cDNA for TaTPP-7A
  misc_feature (1)..(19): sequence corresponding to primer TaTPP1cDNA-F1
  misc_feature (1069)..(1081): sequence corresponding to primer TaTPP1cDNA-R1 (complement)
atggcgaaccaggacgtggtgctccgcccggacatgggcggcatagcggcggccgcggccatgccgggctcctccagccgcgcgatcttcgcgtgccgcggcgccgcgtcctcctccctgcggcgccgctccgccgtcgacgacgagttccgcgccgggtcaccctgcgccacaagctgggtcgtccaggccatgcgggcttcttcgccggcccgctccgcagccgtcgacgagtacgccgcgtggacgaggaagcacccgtcggcccttgctagcttcgagcagatcgcggcggccgccaaggggaagcaggtggtcgtgttcctggactacgacggcacgctctcgcccatcgtcgccgaccccgacacggcggtcatcagcggagagatgcgggaggcagtgcgcggcgttgcgaagcacttcccggcggcgatcgtcaccggccggtgcgtggagaaggtgtgcagcttcgtaggcctctcggagctctactacgcgggcagccacggcatggacatcaaaggcccaggctctaacgcggaggaggtgctcctgcaacctgctcgcgagttcctcccggtcatcgccgaggtctacgaggctctggtggagaagaccaagagcacgccgggggccagggtggaaaacaacaagttctgcctctccgtgcacttccggtgcgtggatgaaaagagatggagtccattggccgagcaagccaaggaggtgctccgggactaccccgatctcagcctcaacgaaggcagaaaggtcctggagatccggccttccatcatgtgggacaagggcaaggccgtggagttcttgctccaatccctgggattcgacggacgcagcgacgtcctgccgctgtacctcggggacgaccggactgacgaggatgctttcaagatgctgagaaagagaggtcatggcctcggcatccttgtctccaagtgccctagggagaccgacgcctcctactctctccaggaccccactgaggtcatggagttccttcaccgcttggtgcagtggaggcgccggcgttcgtcatcgtcggcgatgcgcgcaagagtgtag

SEQ ID NO: 3 - DNA, 2254 bp, Artificial Sequence
  genomic region (gDNA) encoding TaTPP-7A
  5′UTR (1)..(22)
  misc_feature (1)..(18): sequence corresponding to TaTPP-F1 primer
  exon (23)..(270): exon 1
  misc_feature (23)..(39): sequence corresponding to TaTPP-cDNA F1 primer (complement)
  exon (401)..(540): exon 2
  variation (488)..(488): A
  exon (629)..(780): exon 3
  exon (900)..(951): exon 4
  exon (1052)..(1153): exon 5
  exon (1273)..(1350): exon 6
  variation (1300)..(1300): T
  exon (1465)..(1535): exon 7
  exon (1632)..(1701): exon 8
  exon (1845)..(1937): exon 9
  exon (2036)..(2115): exon 10
  misc_feature (2098)..(2115): sequence corresponding to TaTPP-cDNA R1 primer (complement)
  3′UTR (2116)..(2254)
  variation (2144)..(2144): A
  misc_feature (2231)..(2254): sequence corresponding to TaTPP-R1 primer (complement)
cgtgtggttgtttgcgtgcgccatggcgaaccaggacgtggtgctccgcccggacatgggcggcatagcggcggccgcggccatgccgggctcctccagccgcgcgatcttcgcgtgccgcggcgccgcgtcctcctccctgcggcgccgctccgccgtcgacgacgagttccgcgccgggtcaccctgcgccacaagctgggtcgtccaggccatgcgggcttcttcgccggcccgctccgcagccgtcgacgagtacgccgcgtggacggtgagtttggcggctggtttggtgaggcgcgcgtgcccacgcattgctgtgagagcgcgccggccgggctgttttccgtccattctgatgattgatctgcgtttcttttttccttggttggcgcgcgcagaggaagcacccgtcggcccttgctagcttcgagcagatcgcggcggccgccaaggggaagcaggtggtcgtgttcctggactacgacggcacgctctcgcccatcgtcgccgaccccgacacggcggtcatcagcggagaggtaagataagagaccgcgataaccagcctggctagctacttcaagcaacgctcacatcgccgaccatgccttgtccttgatgttctagatgcgggaggcagtgcgcggcgttgcgaagcacttcccggcggcgatcgtcaccggccggtgcgtggagaaggtgtgcagcttcgtaggcctctcggagctctactacgcgggcagccacggcatggacatcaaaggcccaggctctaacgtacgccatctctctcaagcatctctccgtcgcgcaagaatctaactggccatgcatggcccatccctctccaatcattcgtgcctgattccatcaattattcctcgccctatttgcaggcggaggaggtgctcctgcaacctgctcgcgagttcctcccggtcatcgccgaggtacacgacttttgttagttagttgagccacgaggaacttaccaacggcgcatgtactgatctctcgcttttcttggttttattttggtttgggtgccaggtctacgaggctctggtggagaagaccaagagcacgccgggggccagggtggaaaacaacaagttctgcctctccgtgcacttccggtgcgtggatgaaaaggtaaccagagagctcaccaatcaaattaatcagcaacaatctagctaagtcaaacaagaaggttctaagttaatgagagagatcctcatcgatctcaactgctccgatcccctctgcagagatggagtccattggccgagcaagccaaggaggtgctccgggactaccccgatctcagcctcaacgaaggcagaaaggttggttgattggccatggatcgatcatggagttaattactagcagcgcttactaaatcatgttatttaacatttgctcactgagcccgtgaatattgaatctactgggcgtaggtcctggagatccggccttccatcatgtgggacaagggcaaggccgtggagttcttgctccaatccctgggtaggtgcccctgccaccctgcccgctgcgtcgtcttcgtcctcgtcggataaaatgacgggttgcatataaaatctgctgctgtgctgtctctaggattcgacggacgcagcgacgtcctgccgctgtacctcggggacgaccggactgacgaggatgctttcaaggtgcacggattaaactaacacacgtcgcgtccaactgatttcatttgtagttttgtacatatatatgcatgctagctacgaacctagtgatttcatttgttttggctgcttctttatcttctgattttgttgatgtcaactagatgctgagaaagagaggtcatggcctcggcatccttgtctccaagtgccctagggagaccgacgcctcctactctctccaggaccccactgaggtgtatatacatacatatatcatgacgcagatccctgctaagtctagtcatcagtgattgtagcttacatagtattaatttctccttgttttcgtcaggtcatggagttccttcaccgcttggtgcagtggaggcgccggcgttcgtcatcgtcggcgatgcgcgcaagagtgtagcacagtcacagactgccacatcagcggctgctcctggcactgatttatcaagaagcttcagcatgattaattaagtagtttgttagctaatttggttatatcatattccatgcatgtaataaccctcgcctatatctag

SEQ ID NO: 4 - DNA, 18 bp, Artificial Sequence
  forward primer TaTPP-F1
cgtgtggttgtttgcgtg

SEQ ID NO: 5 - DNA, 24 bp, Artificial Sequence
  reverse primer TaTPP-R1
ctagatataggcgagggttattac

SEQ ID NO: 6 - DNA, 17 bp, Artificial Sequence
  forward primer TaTPPcDNA-F1
atggcgaaccaggacgt

SEQ ID NO: 7 - DNA, 18 bp, Artificial Sequence
  reverse primer TaTPPcDNA-R1
ctacactcttgcgcgcat

SEQ ID NO: 8 - DNA, 20 bp, Artificial Sequence
  forward primer QST-TPP-7A-F
ccatgccttgtccttgatgt

SEQ ID NO: 9 - DNA, 22 bp, Artificial Sequence
  reverse primer QST-TPP-7A-R
aaaccaagaaaagcgagagatc

SEQ ID NO: 10 - DNA, 25 bp, Artificial Sequence
  forward primer (cloning) TPP-TaA-F
cgggatccatggcgaaccaggacgt

SEQ ID NO: 11 - DNA, 26 bp, Artificial Sequence
  reverse primer (cloning) TPP-TaA-R
cggaattcctacactcttgcgcgcat

SEQ ID NO: 12 - DNA, 20 bp, Artificial Sequence
  forward primer TPP-P-1F (promoter amplication)
gaatgtagcagtccacctat

SEQ ID NO: 13 - DNA, 20 bp, Artificial Sequence
  reverse primer TPP-P-1R (promoter amplication)
acgcagatcaatcatcagaa

SEQ ID NO: 14 - DNA, 2497 bp, Artificial Sequence
  TPP-7A promoter  version  1
  variation (409)..(410): tg is tcg in promoter version 2 SNP409/410
  variation (493)..(493): t is c in promoter version 2 SNP493
  variation (1208)..(1208): A is G in promoter version 2 SNP1208
  variation (1708)..(1708): T is G in promoter version 2 SNP1708
  variation (1980)..(1980): G is A in promoter version 2 SNP1980
  misc_feature (2472)..(2472): transcription start site
gaatgtagcagtccacctattgcccctaagcccaacaatcatgtgagctttacaatcatgacacaacatcctaatcttttgacgtttgtctatcacatccatttcaacctcatccttcttgctagatgagatcccttatgttgtgatcccttttgaagcactactagtctcaaccctatacttgttggcttccctgtgtggacacactggaaaacactcttgataagtgtcggaggacctttcttctagccattgttagaagcagctatgtgtgtggcgaatcccaatttgaaggcgtagtcattttagaattttttagcatcatcaatcgagtcgaactctatgccaacatatggtgcaagcagttgggatcaaatattgccatcttcttccttttcatgcattgtgtggtagcattgagcatgggatctgaatccacgccaccgtgagttgtaaaatgctctcctcacctcctatcccaccttcggtatcttactaacattgcaacaaaagtagaacatcattcatcaatagcatttagaacaaaaggtagaacataattcctttttcggtgcaaactttgtttaggtagaaacaataagatcagagagggccagagtaaccctctattttggcaacaaacatttatagaccaacactatcaattgaacattccagattggtggatttctttagtagagcgtgcacaatttgctatttttagatagccatgctcaatgaaagtaaacaacatttgctacaacattctcattttctgaagactaggtctatttctgcaacatcatgtttttgaaggattttccattcatcaaaatatatcattgttacttttgcatcctcacttttttccaaaacaaatactaggtttagagaagggagacaaaaatgttttctgggtgcacttttttgaattgggtttttgcgctttcctgaatcataacatataaatttgaatgggagccccctctgctactggtattcaacatcaacatatagaaaattgaactttgtaactttcaatgaattctacatcaatggggatgcaaattgctgaattgaacattaacattcagctaattcggctcaccaacattcaattttttttagctcatcaacattcataaaatttcaggtcttgcaacttactgaattcaacatcatctttgttgggatgcaactatttactaaattcaacattaacattcggtaaattcatatatgcatgcaacaaggaaattgtttctcaggaaaaaaactaggttcatagaaaccaacatatcaaatccaatgttgctaggcagcctcccacttgctagagcttaattcgtcggccagggaatagtcaacccctaagagctttgagccatggcgtaggagagcatatctatgacgatatggtgcgccacctggccagattaggggcggggtgcaagggaggtactatgggtgccagtgttgcaatggtggtgtagggatgttgcggcggacaccggtcatagattctggatcccttaccgcggggcaaaaggggccgccactcacgattcacacgcatgggggaatgcggtttggaggctggttgttgtgttccaacacgtcgtctgttgcaacggtcaagttgggggaggaggctgcgcgtggtccagcttgcggcaaatcggacgactccgcagcacctgatctaactgctcgcatgagagcttacttttggcatgcatcacagccacgataaaacaaggctaacatagtcttggtccatctataatacatgttggaccatgcttctctctccccactaatcgattgctttctcctttgaccgtatttgatcttattttttcttctagtattttattttctcttgacattgggtttattggatgtgcgcggctcccgcatgtcagtgaccaacatcaaaggacactccttccgccaaagtccctctgattcttcgagtcgattttcccccttgcaacagatggctatatgtgactgatcgagaaatggccacacatttcatccaaaaatgaagaatatttgaatttccacagcctccagagcaccactttgatttgaactcgaaatatgaatatagtaaaagggtctacatataatttgaaagtattttgcaggacaaaaacaacaatgttattctcgaatcttaatctatagtcgtcaattaaattttcagaatgttaactgttcatataattgtgcaccctgcaattgtgaatgagaaaacgacacatgtccactccggttagaaaaaacgcagtagttccactagtatgggtaccgacccaacgccgctccgcctttataagtaccgacacttcgccattggcttcttcacccagggaaaaccggtctggtcgcctcttcctcgtgaactattccccactgtcactgctgggcaccactctcacgcagcgggaggcgcgtgtcgtgagcacacgtgtggttgtttgcgtgcgcc

SEQ ID NO: 15 - DNA, 2498 bp, Artificial Sequence
  TPP-7A promoter version 2
  variation (409)..(411): TCG is TG in promoter version 1  SNP 409/410
  variation (494)..(494): C is T in promoter version 1  SNP 409/410
  variation (1209)..(1209): G is A in promoter version 1  SNP 1208
  variation (1709)..(1709): G is T in promoter version 1  SNP 1708
  variation (1981)..(1981): A is G in promoter version 1  SNP 1980
  misc_feature (2473)..(2473): Transcription start site
gaatgtagcagtccacctattgcccctaagcccaacaatcatgtgagctttacaatcatgacacaacatcctaatcttttgacgtttgtctatcacatccatttcaacctcatccttcttgctagatgagatcccttatgttgtgatcccttttgaagcactactagtctcaaccctatacttgttggcttccctgtgtggacacactggaaaacactcttgataagtgtcggaggacctttcttctagccattgttagaagcagctatgtgtgtggcgaatcccaatttgaaggcgtagtcattttagaattttttagcatcatcaatcgagtcgaactctatgccaacatatggtgcaagcagttgggatcaaatattgccatcttcttccttttcatgcattgtgtcggtagcattgagcatgggatctgaatccacgccaccgtgagttgtaaaatgctctcctcacctcctatcccaccttcggtatcctactaacattgcaacaaaagtagaacatcattcatcaatagcatttagaacaaaaggtagaacataattcctttttcggtgcaaactttgtttaggtagaaacaataagatcagagagggccagagtaaccctctattttggcaacaaacatttatagaccaacactatcaattgaacattccagattggtggatttctttagtagagcgtgcacaatttgctatttttagatagccatgctcaatgaaagtaaacaacatttgctacaacattctcattttctgaagactaggtctatttctgcaacatcatgtttttgaaggattttccattcatcaaaatatatcattgttacttttgcatcctcacttttttccaaaacaaatactaggtttagagaagggagacaaaaatgttttctgggtgcacttttttgaattgggtttttgcgctttcctgaatcataacatataaatttgaatgggagccccctctgctactggtattcaacatcaacatatagaaaattgaactttgtaactttcaatgaattctacatcaatggggatgcaaattgctgaattgaacattaacattcagctaattcggctcaccaacattcaattttttttagctcatcaacattcataaaatttcaggtcttgcaacttactgaattcaacatcatctttgttgggatgcaactatttactgaattcaacattaacattcggtaaattcatatatgcatgcaacaaggaaattgtttctcaggaaaaaaactaggttcatagaaaccaacatatcaaatccaatgttgctaggcagcctcccacttgctagagcttaattcgtcggccagggaatagtcaacccctaagagctttgagccatggcgtaggagagcatatctatgacgatatggtgcgccacctggccagattaggggcggggtgcaagggaggtactatgggtgccagtgttgcaatggtggtgtagggatgttgcggcggacaccggtcatagattctggatcccttaccgcggggcaaaaggggccgccactcacgattcacacgcatgggggaatgcggtttggaggctggttgttgtgttccaacacgtcgtctgttgcaacggtcaagttgggggaggaggctgcgcgtggtccagcttgcggcaaatcggacgactccgcagcacctgatctaacggctcgcatgagagcttacttttggcatgcatcacagccacgataaaacaaggctaacatagtcttggtccatctataatacatgttggaccatgcttctctctccccactaatcgattgctttctcctttgaccgtatttgatcttattttttcttctagtattttattttctcttgacattgggtttattggatgtgcgcggctcccgcatgtcagtgaccaacatcaaaggacactccttccgccaaagtccctctgattcttcgagtcaattttcccccttgcaacagatggctatatgtgactgatcgagaaatggccacacatttcatccaaaaatgaagaatatttgaatttccacagcctccagagcaccactttgatttgaactcgaaatatgaatatagtaaaagggtctacatataatttgaaagtattttgcaggacaaaaacaacaatgttattctcgaatcttaatctatagtcgtcaattaaattttcagaatgttaactgttcatataattgtgcaccctgcaattgtgaatgagaaaacgacacatgtccactccggttagaaaaaacgcagtagttccactagtatgggtaccgacccaacgccgctccgcctttataagtaccgacacttcgccattggcttcttcacccagggaaaaccggtctggtcgcctcttcctcgtgaactattccccactgtcactgctgggcaccactctcacgcagcgggaggcgcgtgtcgtgagcacacgtgtggttgtttgcgtgcgcc

SEQ ID NO: 16 - DNA, 54 bp, Artificial Sequence
  forward primer TPP-P-TF
ggggacaagtttgtacaaaaaagcaggcttcctcttgataagtgtcggaggacc

SEQ ID NO: 17 - DNA, 47 bp, Artificial Sequence
  reverse primer TPP-P-TR
ggggaccactttgtacaagaaagctgggtcggcgcacgcaaacaacc

SEQ ID NO: 18 - DNA, 1488 bp, Artificial Sequence
  nucleotide sequence between SNP493 and SNP1980 of promoter version 1 as in SEQ ID No. 14
ttactaacattgcaacaaaagtagaacatcattcatcaatagcatttagaacaaaaggtagaacataattcctttttcggtgcaaactttgtttaggtagaaacaataagatcagagagggccagagtaaccctctattttggcaacaaacatttatagaccaacactatcaattgaacattccagattggtggatttctttagtagagcgtgcacaatttgctatttttagatagccatgctcaatgaaagtaaacaacatttgctacaacattctcattttctgaagactaggtctatttctgcaacatcatgtttttgaaggattttccattcatcaaaatatatcattgttacttttgcatcctcacttttttccaaaacaaatactaggtttagagaagggagacaaaaatgttttctgggtgcacttttttgaattgggtttttgcgctttcctgaatcataacatataaatttgaatgggagccccctctgctactggtattcaacatcaacatatagaaaattgaactttgtaactttcaatgaattctacatcaatggggatgcaaattgctgaattgaacattaacattcagctaattcggctcaccaacattcaattttttttagctcatcaacattcataaaatttcaggtcttgcaacttactgaattcaacatcatctttgttgggatgcaactatttactaaattcaacattaacattcggtaaattcatatatgcatgcaacaaggaaattgtttctcaggaaaaaaactaggttcatagaaaccaacatatcaaatccaatgttgctaggcagcctcccacttgctagagcttaattcgtcggccagggaatagtcaacccctaagagctttgagccatggcgtaggagagcatatctatgacgatatggtgcgccacctggccagattaggggcggggtgcaagggaggtactatgggtgccagtgttgcaatggtggtgtagggatgttgcggcggacaccggtcatagattctggatcccttaccgcggggcaaaaggggccgccactcacgattcacacgcatgggggaatgcggtttggaggctggttgttgtgttccaacacgtcgtctgttgcaacggtcaagttgggggaggaggctgcgcgtggtccagcttgcggcaaatcggacgactccgcagcacctgatctaactgctcgcatgagagcttacttttggcatgcatcacagccacgataaaacaaggctaacatagtcttggtccatctataatacatgttggaccatgcttctctctccccactaatcgattgctttctcctttgaccgtatttgatcttattttttcttctagtattttattttctcttgacattgggtttattggatgtgcgcggctcccgcatgtcagtgaccaacatcaaaggacactccttccgccaaagtccctctgattcttcgagtcg

SEQ ID NO: 19 - DNA, 48 bp, Artificial Sequence
  nucleotide sequence  between positions 467-514 of the 5′ end  of of PCR amplification TaTPP version 1
ggtcgtgttcctggactacgacggcacgctctcgcccatcgtcgccga

SEQ ID NO: 20 - DNA, 48 bp, Artificial Sequence
  nucleotide sequence  between positions 467-514 of the 5′ end  of of PCR amplification TaTPP version 2
ggtcgtgttcctggactacgaaggcacgctctcgcccatcgtcgccga

SEQ ID NO: 21 - DNA, 43 bp, Artificial Sequence
  nucleotide sequence of KASP based primer 488F1
gaaggtgaccaagttcatgctggtcgtgttcctggactacgac

SEQ ID NO: 22 - DNA, 43 bp, Artificial Sequence
  nucleotide sequence of KASP based primer 488F2
gaaggtcggagtcaacggattggtcgtgttcctggactacgaa

SEQ ID NO: 23 - DNA, 23 bp, Artificial Sequence
  nucleotide sequence of KASP primer 488C
tcggcgacgatgggcgagagcgt

SEQ ID NO: 24 - DNA, 48 bp, Artificial Sequence
  nucleotide sequence of SNP 488 marker
  misc_feature (22)..(22): N is a or c
ggtcgtgttcctggactacganggcacgctctcgcccatcgtcgccga

SEQ ID NO: 25 - DNA, 48 bp, Artificial Sequence
  nucleotide sequence  between positions 2121-2168 of the 5′ end of of PCR amplification TaTPP version 1
tcacagactgccacatcagcggctgctcctggcactgatttatcaaga

SEQ ID NO: 26 - DNA, 48 bp, Artificial Sequence
  nucleotide sequence  between positions 2121-2168 of the 5′ end of of PCR amplification TaTPP version 2
tcacagactgccacatcagcggcagctcctggcactgatttatcaaga

SEQ ID NO: 27 - DNA, 45 bp, Artificial Sequence
  nucleotide sequence of KASP based primer 2144F1
gaaggtgaccaagttcatgcttcacagactgccacatcagcggct

SEQ ID NO: 28 - DNA, 45 bp, Artificial Sequence
  nucleotide sequence of KASP based primer 2144F2
gaaggtcggagtcaacggatttcacagactgccacatcagcggca

SEQ ID NO: 29 - DNA, 23 bp, Artificial Sequence
  nucleotide sequence of KASP primer 2144C
tcttgataaatcagtgccaggag

SEQ ID NO: 30 - DNA, 48 bp, Artificial Sequence
  nucleotide sequence of SNP 2144C marker
  misc_feature (24)..(24): n is a or t
tcacagactgccacatcagcggcngctcctggcactgatttatcaaga

(19) United States
(12) Patent Application Publication
(10) Pub. No.: US 2019/0330649 A1
(43) Pub. Date: Oct. 31, 2019

(54) Plant Grain Trait-Related Protein, Gene, Promoter and SNPS and Haplotypes

(71) Applicant: INSTITUTE OF CROP SCIENCES, THE CHINESE ACADEMY OF AGRICULTURAL SCIENCES, Beijing (CN)

(72) Inventors: Xueyong ZHANG, Beijing (CN); Hongxia LIU, Beijing (CN)

(21) Appl. No.: 16/474,660

(22) PCT Filed: Dec. 20, 2017
(86) PCT No.: PCT/CN2017/117519

§ 371 Date: Jun. 28, 2019

(30) Foreign Application Priority Data
Dec. 21, 2016 (CN) 201611190833.4
Dec. 21, 2016 (CN) 201611195844.1

(51) Int. Cl. C12N 15/82; C12N 9/16; C12Q 1/6895

(52) U.S. Cl. CPC C12N 15/8251; C12N 9/16; C12Y 301/03012; C12Q 2600/156; C12Q 1/6895; C12Q 2600/13; C12N 15/8262

(57) ABSTRACT

The present invention discloses a plant grain trait-related protein as well as a coding gene and use thereof. The present invention provides protein TaTPP-7A, which is a protein consisting of the amino acid sequence as shown by SEQ ID NO: 1 in Sequence Listing. The gene encoding the protein TaTPP-7A is also within the protection scope of the present invention. The present invention is further directed to a method of cultivating transgenic plants, comprising the step of introducing the gene TaTPP-7A into a starting plant to obtain a transgenic plant; said transgenic plant satisfies at least one of the following (e1) to (e6):(e1) having a heavier thousand-kernel weight in grains than said starting plant; (e2) having a heavier kernel weight in grains than said starting plant; (e3) having a larger size in grains than said starting plant; (e4) having a longer kernel length in grains than said starting plant; (e5) having a wider kernel width in grains than said starting plant; (e6) having a thicker kernel thickness in grains than said starting plant. Therefore, the protein and coding gene thereof provided by the present invention can be used for improving the quality of plants and increasing the yield of plant gains, and has broad application prospects. The invention also provides for SNP markers and haplotypes associated with the above grain characteristics.

### TECHNICAL FIELD

The present invention relates to a plant grain trait-related protein encoding trehalose-6 phosphate phosphatase (TPP) as well as a coding gene from wheat (TaTPP) and use thereof to modify grain traits, such as increasing grain length, grain width, thousand grain weight, spike length, grain number and ultimately grain yield. The present invention also provides single nucleotide polymorphism (SNP) markers, associated with increased grain length, width and thousand grain or kernel weight, both in the TPP coding region, as well as in the promoter region. The invention also provides promoter regions, and identified the stronger promoter region associated with increase in grain length, grain width and thousand grain weight, which can be used to increase expression in cereal plants, such as wheat, of any coding region of interest. The invention further identifies haplotypes favorable to increase in grain length, grain width, thousand grain weight, and ultimately yield in cereals such as wheat.

### BACKGROUND ART

Wheat is one of the important food crops in China and worldwide, and it directly affects humans' living standard and the national food security. It has always been the long-term pursuit of wheat breeders in China to improve the yield of wheat per unit and allow a high and stable output. The desire to increase wheat yield contrast with conflicting circumstances such as increasingly decreased food planting areas, land desertification, salinization, global warming and ever-increasing population base. Accordingly, ways to improve or increase the yield of wheat per unit and solve the growing demand for food has become a more and more prominent and important task in breeding. Therefore, the use of molecular biology techniques in cloning functional genes associated with the yield of wheat, and further in-depth analysis of the function thereof can provide important reference gene resources for developing markers in wheat molecular marker-assisted breeding, and are of great significance in both science and practical application for accelerating the process of wheat breeding in China and improving China's wheat yield.

Kernel weight is one of the three elements of yield, and the key factors that determine kernel weight include grain shape and grain filling rate. In the practice of grain production as well as in breeding, thousand-kernel weight is often used as an indicator of grain size, the latter itself mainly composed of grain-type trait parameters (such as kernel length, kernel width and kernel thickness) as well as a positive indicator of yield.

### SUMMARY OF THE INVENTION

The invention provides for a protein having trehalose-6 phosphate phosphatase enzymatic activity selected from:
a. a protein comprising the amino acid sequence of SEQ ID NO: 1;
b. a protein comprising an amino acid sequence having at least 90% sequence identity to the amino acid sequence of SEQ ID No: 1;
c. a protein comprising the amino acid sequence of SEQ ID NO: 1 wherein one or more amino acid residues are substituted or deleted or inserted, and wherein the presence of the protein is associated with increased grain length, grain width or increased thousand kernel weight, such as a protein according to SEQ ID No: 1, wherein the Asp residue at position 112 is substituted by a Glu residue, and/or wherein the Ala residue at position 241 is substituted by a Val residue.

In another embodiment the invention provides a nucleic acid, such as a DNA or RNA molecule comprising a nucleotide sequence encoding the protein according to claim 1. The nucleic acid may be selected from:
a. a nucleic acid, such as a DNA molecule, comprising the nucleotide sequence of SEQ ID NO: 2;
b. a nucleic acid, such as a DNA molecule, comprising the nucleotide sequence of SEQ ID NO: 3 from nucleotide positions 23 to nucleotide position 2115;
c. a nucleic acid, such as a DNA molecule, comprising the nucleotide sequence of SEQ ID NO: 3
d. a nucleic acid, such as a DNA molecule, which hybridizes with a DNA molecule according to any one of a to c above under stringent conditions and codes for a protein according to claim 1;
e. a nucleic acid, such as a DNA molecule which comprises a nucleotide sequence having at least 90% sequence identity to the nucleotide sequence of SEQ ID NO: 3 from nucleotide positions 23 to nucleotide position 2115 or the nucleotide sequence of SEQ ID NO: 2.

In yet another embodiment, the invention provides a recombinant expression cassette comprising the following operably linked DNA elements
a. a plant-expressible promoter, such as a heterologous plant expressible promoter
b. A DNA region encoding a protein according to claim 1 or a DNA region according to claim 2;
c. a DNA region which is a transcription termination and polyadenylation region, such as a transcription termination and polyadenylation region functional in plants.

The invention also provides a recombinant expression vector, transgenic cell line, transgenic plant tissue, transgenic plant or recombinant strain, or grain or seed containing the a nucleic acid as herein described or a recombinant expression cassette as herein described. The plant may be a cereal plant, such as a wheat plant.

In yet another embodiment the invention provides the use of a protein as herein described for:

a. regulating the size of plant grains, such as increase or decrease the grain length or grain width, particularly of grains of wheat plants;

b. increasing the size of plant grains, particularly of grains of wheat plants;

c. regulating the thousand-kernel weight of plant grains such as increase or decrease the grain length or grain width, particularly of grains of wheat plants;

d. increasing the thousand-kernel weight, particularly of grains of wheat plants;

e. regulating the kernel weight of plant grains such as increase or decrease the grain length or grain width, particularly of grains of wheat plants;

f. increasing the kernel weight of plant grains, particularly of wheat grains;

g. regulating the kernel length of plant grains such as increase or decrease the grain length or grain width, particularly of grains of wheat plants;

h. increasing the kernel length of plant grains particularly of grains of wheat plants;

i. regulating the kernel width of plant grains such as increase or decrease the grain length or grain width, particularly of grains of wheat plants;

j. increasing the kernel width of plant grains particularly of grains of wheat plants;

k. regulating the kernel thickness of plant grains such as increase or decrease the grain length or grain width, particularly of grains of wheat plants;

l. increasing the kernel thickness of plant grains particularly of grains of wheat plants;

m. increasing the tiller length of plants, particularly of cereal plants such as wheat;

n. increasing the spike length of plants, particularly of cereal plants such as wheat;

o. increasing the grain yield of plants, such as cereal plants, such as wheat.

In another embodiment, a method is provided of producing plants, such as cereal plants, including wheat plants, comprising the step of

a) increasing the level and/or activity of a protein as herein described; or

b) increasing the expression of a nucleic acid as herein described in a plant cell or plant

c) introducing a recombinant expression cassette as herein described into a plant cell or a plant, to obtain a transgenic plant,

wherein the plant has

1) an increased thousand-kernel weight in grains than said starting plant or a control plant;

2) an increased kernel weight in grains than said starting plant or control plant;

3) a larger size in grains than said starting plant or control plant;

4) a longer kernel length in grains than said starting plant or control plant;

5) a wider kernel width in grains than said starting plant or control plant;

6) a thicker kernel thickness in grains than said starting plant or control plant;

7) an increased tiller length than said starting plant or control plant;

8) an increased spike length than said starting plant or control plant;

9) an increased grain number than said starting plant or control plant; or

10) an increased grain yield than said starting plant or control plant.

The invention also provides a method to

(1) increase thousand-kernel weight in grains;

(2) increase kernel weight in grains;

(3) increase size in grains;

(4) increase length in grains;

(5) increase width in grains;

(6) increase thickness in grains;

57) increase tiller length in plants;

(8) increase spike length in plants;

(9) increase grain number in plants; or

(10) increase grain yield in plants

comprising the step of increasing the content or activity of the protein as herein described in the plant, such as a cereal plant, including a wheat plant.

In another aspect of the invention, an isolated promoter region comprising the nucleotide sequence of SEQ ID No:14 or SEQ ID No: 15 or a nucleotide sequence comprising at least 90%, 95% or 99% sequence identity thereto is provided.

In yet another embodiment, the invention provides a recombinant gene comprising the following operably linked DNA fragments:
a. a promoter region as herein described;
b. a DNA region encoding an RNA molecule or a protein of interest
c. a transcription termination and polyadenylation region functional in plant cells.

Also provided is a plant, such as a cereal plant, including a wheat plant comprising the recombinant gene of the invention.

In yet another embodiment, the invention provides a method for identifying or assisting in identifying wheat grain traits, such as thousand kernel weight of wheat grains, or kernel length of wheat grains comprising the step of:

detecting whether the genotype based on 488 SNP site in the genomic DNA of the wheat to be tested is AA genotype, AC genotype or CC genotype; the wheat of AA genotype has better grain traits than the wheat of CC genotype;

the better grain traits are shown as higher thousand-kernel weight and/or longer kernel length;

the 488 SNP site refers to the nucleotide at position 22 from 5′ end of SEQ ID NO: 24.

The invention also provides the use of a material for detecting the genotype based on 488 SNP site in the genomic DNA of wheat, for identifying or assisting in identifying wheat grain traits; the grain traits being thousand-kernel weight and/or kernel length, as well as a primer set I, which consists of 488F1, 488F2 and 488C;

said primer 488F1 is (b1) or (b2) as follows:

(b1) a single-stranded DNA molecule as shown by SEQ ID NO:21;

(b2) a DNA molecule obtained by subjecting SEQ ID NO: 21 to substitution and/or deletion and/or addition of one or several nucleotides and having the same function as SEQ ID NO:21;

said primer 488F2 is (b3) or (b4) as follows:

(b3) a single-stranded DNA molecule as shown by SEQ ID NO:22

(b4) a DNA molecule obtained by subjecting SEQ ID NO: 22 to substitution and/or deletion and/or addition of one or several nucleotides and having the same function as SEQ ID NO:22;

said primer 488C is (b5) or (b6) as follows:

(b5) a single-stranded DNA molecule as shown by SEQ ID NO:23;

(b6) a DNA molecule obtained by subjecting SEQ ID NO:23 to substitution and/or deletion and/or addition of one or several nucleotides and having the same function as SEQ ID NO:23.

In yet another embodiment, the invention provides a method for identifying or assisting in identifying wheat grain traits, such as thousand kernel weight or kernel length, comprising the step of:

detecting whether the genotype based on 2144 SNP site in the genomic DNA of the wheat to be tested is AA genotype, AT genotype or TT genotype; the wheat of AA genotype has better grain traits than the wheat of TT genotype;

the better grain traits are shown as higher thousand-kernel weight and/or longer kernel length;

the 2144 SNP site refers to the nucleotide at position 24 from 5′ end of SEQ ID NO: 30.

The invention also provides a primer set I, which consists of 2144F1, 2144F2 and 2144C;

said primer 2144F1 is (b1) or (b2) as follows:

(b1) a single-stranded DNA molecule as shown by SEQ ID NO:27;

(b2) a DNA molecule obtained by subjecting SEQ ID NO: 27 to substitution and/or deletion and/or addition of one or several nucleotides and having the same function as SEQ ID NO:21;

said primer 2144F2 is (b3) or (b4) as follows:

(b3) a single-stranded DNA molecule as shown by SEQ ID NO:28

(b4) a DNA molecule obtained by subjecting SEQ ID NO: 28 to substitution and/or deletion and/or addition of one or several nucleotides and having the same function as SEQ ID NO:22;

said primer 2144C is (b5) or (b6) as follows:

(b5) a single-stranded DNA molecule as shown by SEQ ID NO:29;

(b6) a DNA molecule obtained by subjecting SEQ ID NO:29 to substitution and/or deletion and/or addition of one or several nucleotides and having the same function as SEQ ID NO:29 and use thereof for identifying or assisting in identifying wheat grain traits; the grain traits being thousand-kernel weight and/or kernel length; or

for identifying or assisting in identifying the thousand-kernel weight of wheat grains; or

for identifying or assisting in identifying the kernel length of wheat grains;

The invention also provides a method for obtaining a wheat plant with

(1) increased thousand-kernel weight in grains;

(2) increased kernel weight in grains;

(3) increased size in grains;

(4) increased length in grains;

(5) increased width in grains;

(6) increased thickness in grains;

57) increased tiller length in plants;

(8) increased spike length in plants;

(9) increased grain number in plants; or

(10) increased grain yield in plants

comprising the step of selecting a wheat plant with haplotype Hap I.

### DESCRIPTION OF DRAWINGS

FIG. 1: Grain characteristics of grains from wheat lines wherein TPP expression is increased through overexpression of TaTPP chimeric gene (TaTPP-OE), or wheat lines wherein TPP expression is decreased through a chimeric gene expressing silencing RNA (TaTPP-RNAi). Panel A. Effect of overexpression of TaTPP in wheat on grains. TaTPP5-3; TaTPP-10-4 and TaTPP-13-7 are TPP overexpressing lines. Negative control: untransformed wheat variety Fielder. Panel B. Effect of overexpression or reducing expressing of TPP in wheat on the grain length. TaTPP-OE: grain from transgenic wheat line overexpressing TaTPP. TaTPP-RNai: grain from transgenic wheat line wherein expression of TPP is reduced through silencing RNA.

FIG. 2 shows the average kernel length and average thousand-kernel weight of grains in each transgenic wheat line. Panel A: average grain length (GL) (cm) of transgenic TPP overexpressing lines TaTPP5-3, TaTPP-10-4 and TaTPP-13-7. NTCK: untransformed fielder. Panel B: Thousand grain weight (g) of grains from transgenic lines and control line as in panel A. Panel C: graphic representation of thousand kernel weight (TKW) (in gram left Y-axis), grain length (GL) and grain weight (GW) (in cm—right Y-axis) for wild type control wheat line (WT—left bar), TPP overexpressing wheat lines (TPO—middle bar), TPP reduced expression wheat lines (TPR—right bar). For TKW and GL, there is a statistically significant difference for average TKW and GL both between WT and TPO, TPO and TPR and WT and TPR lines. For GW, there is a statistically significant difference between the TPO and WT and the TPO and TPR lines.

FIG. 3 shows the effect of increase (TPO) or decrease (TPR) of TPP expression in wheat compared to wild type wheat line (Fielder, WT) on lemma length, width, as well as palea length and palea width. Panel A. visual representation of palea and lemma of the different transgenic lines. Panel B. Graphic representation of lemma length (mm) lemma width (mm), palea length (mm) and palea width for wild type control wheat line (WT—left bar), TPP overexpressing wheat lines (TPO—middle bar), TPP reduced expression wheat lines (TPR—right bar). For lemma and palea length there is a statistically significant difference between WT and TPR, as well as between TPO and TPR lines. For lemma and palea width there is a statistically significant difference between TPO and both WT and TPR lines.

FIG. 4 shows the effect of increase (TPO lines) or decrease (TPR lines) of TPP expression in wheat on spike length and tiller length. Lane 1: Fielder; Lane 2: TPR 47-1-1; Lane 3: TPR 7-2-3; Lane 4: TPR-68-12-4; Lane 5: TPO-6-5-3; Lane 6: TPO-5-4-2; Lane 7: TPO-14-3-9.

FIG. 5 shows the effect of TaTPP overexpression in transgenic Arabidopsis lines (TaTPP-OE) on growth and development in comparison to untransformed WT Arabidopsis lines (Panel A) as well as on pod size and morphology (Panel B) and grain size and morphology (Panel C).

FIG. 6 is a graphic representation of the TaTPP promoter region and coding region (genomic) with an indication of the different SNPs. Due to the use of difference reference points in the nucleotide sequences, the SNP at position −2090 corresponds to SNP 409/410, SNP at position −2006 corresponds to SNP 493, the SNP at position −1291 corresponds to SNP 1208, the SNP at position −783 corresponds to SNP 1708, the SNP at position −511 corresponds to position corresponds to SNP 1980, the SNP at position +466 corresponds to SNP 488, the SNP at position 1278 corresponds to position 1300 and the SNP at position 2122 corresponds to SNP 2144. The boxes correspond to TaTPP-7A exons (for nucleotide and positions of the exons see SEQ ID No. 3). For the nucleotide sequence of the promoter region(s) see SEQ ID Nos 14 and 15. ATG: start codon; TSS: transcription start site; TAG: translation stop codon; polyA: polyadenylation site. Hap I, Hap II and Hap III represent frequently occurring haplotypes in wheat and indicate the nucleotides of the SNP present at the different SNP positions in the different haplotypes which occur together.

FIG. 7. Expression of luciferase under control of the TaTPP promoter of HapI (Luc-HapI P; SEQ ID No 14) and of HapII (Luc-HapII P; SEQ ID No 15) in Nicotiana tabacum compared to transgenic tobacco transformed with an empty vector (LUC-EV). Panel A: Fluorescence image and average values. Panel B: fluorescence in leaves at different stages. As can be seen, the HapI promoter is significantly stronger in expressing than the HapII promoter (about 3 times stronger).

FIG. 8. Panel A. Relative occurrence of the different haplotypes Hap I, Hap II and Hap III in Chinese wheat varieties developed in history. Whereas in the 1930s all Chinese varieties analyzed had Hap II haplotype (middle bar), from the 1940s on, the relative occurrence of Hap I haplotype increased steadily (left bar) while HapII (middle bar) and Hap III occurrence gradually decreased. This correlated with the increase in Thousand Kernel Weight (indicated by the dashed line) over time. Panel B. Geographic distribution of the different Haplotypes. In China, the majority of the analyzed wheat lines exhibit Hap I haplotype. In the Russian Federation, the Hap I haplotype is also predominantly present, but Hap III presence is also significant, and even Hap II is represented. In North and Middle America, Europe and Australia, the predominant haplotype of the analyzed lines is Hap III, with only a minor relative occurrence of HapI.

### VARIOUS DEFINITIONS

TaTPP genes in related monocot species or in other cultivars or varieties can also be identified using hybridization with a probe having the nucleotide sequence of an TaTPP gene or part thereof. Stringent hybridization conditions, such as those described below, can be used to identify nucleotide sequences, which are substantially identical to a given nucleotide sequence. For example, TaTPPC genes from other monocot species than the specific sequences disclosed herein are said to be substantially identical or essentially similar if they can be detected by hybridization under stringent, preferably highly stringent conditions. Stringent conditions are sequence dependent and will be different in different circumstances. Generally, stringent conditions are selected to be about 5° C. lower than the thermal melting point (Tm) for the specific sequences at a defined ionic strength and pH. The Tm is the temperature (under defined ionic strength and pH) at which 50% of the target sequence hybridizes to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridizations (Northern blots using a probe of e.g. 100 nt) are for example those which include at least one wash in 0.2×SSC at 63° C. for 20 min, or equivalent conditions.

“High stringency conditions” can be provided, for example, by hybridization at 65° C. in an aqueous solution containing 6×SSC (20×SSC contains 3.0 M NaCl, 0.3 M Na-citrate, pH 7.0), 5×Denhardt's (100×Denhardt's contains 2% Ficoll, 2% Polyvinyl pyrollidone, 2% Bovine Serum Albumin), 0.5% sodium dodecyl sulphate (SDS), and 20 μg/ml denaturated carrier DNA (single-stranded fish sperm DNA, with an average length of 120-3000 nucleotides) as non-specific competitor. Following hybridization, high stringency washing may be done in several steps, with a final wash (about 30 min) at the hybridization temperature in 0.2-0.1×SSC, 0.1% SDS.

“Moderate stringency conditions” refers to conditions equivalent to hybridization in the above described solution but at about 60-62° C. Moderate stringency washing may be done at the hybridization temperature in 1×SSC, 0.1% SDS.

“Low stringency” refers to conditions equivalent to hybridization in the above described solution at about 50-52° C. Low stringency washing may be done at the hybridization temperature in 2×SSC, 0.1% SDS. See also Sambrook et al. (1989) and Sambrook and Russell (2001).

Monocot plants, also known as monocotyledons or monocotelydon plants, are well known in the art and are plants which have one cotyledon in their seeds. Monocot plants comprise Oryza sp. (including rice), Zea sp. (including maize), Saccharum sp. (including sugarcane), Triticum sp. (including wheat), Hordeum, Secale, Avena, Lolium, Festuca Brachypodium distachion, Musa sp. (including banana).

The terms “expressing in said plant” as well as “expressing in a plant, plant part, plant organ or plant cell” as used throughout the present application relate to the occurrence of an expression product of a nucleic acid resulting from transcription of said nucleic acid. In connection with some embodiments of the methods according to the invention, the term may additionally include introducing a chimeric gene comprising the nucleic acid to be expressed in the plant.

A chimeric gene is an artificial gene constructed by operably linking fragments of unrelated genes or other nucleic acid sequences. In other words “chimeric gene” denotes a gene which is not normally found in a plant species or refers to any gene in which the promoter or one or more other regulatory regions of the gene are not associated in nature with a part or all of the transcribed nucleic acid, i. e. are heterologous with respect to the transcribed nucleic acid. The term “heterologous” refers to the relationship between two or more nucleic acid or protein sequences that are derived from different sources. For example, a promoter is heterologous with respect to an operably linked nucleic acid sequence, such as a coding sequence, if such a combination is not normally found in nature. In addition, a particular sequence may be “heterologous” with respect to a cell or organism into which it is inserted (i.e. does not naturally occur in that particular cell or organism). For example, the chimeric gene disclosed herein is a heterologous nucleic acid.

The chimeric gene may also comprise a transcription termination or polyadenylation sequence functional in a plant cell, particularly a monocot, more preferably a cereal or wheat plant cell. As a transcription termination or polyadenylation sequence, use may be made of any corresponding sequence of bacterial origin, such as for example the nos terminator of Agrobacterium tumefaciens, of viral origin, such as for example the CaMV 35S terminator, or of plant origin, such as for example a histone terminator as described in published Patent Application EP 0 633 317 A1.

Increasing the expression and/or activity of the TATPP protein can be increasing the amount of (functional) TATPP protein produced or increasing the expression and/or activity of TATPP. Said increase in the amount of (functional) TATPP protein produced can be an increase of at least 2-fold, 4-fold, 10-fold, 25-fold, 50-fold, 75-fold, 100-fold or even more as compared to the amount of (functional) TATPP protein produced by a cell with wild type TATPP expression levels. Said increase in expression and/or activity can be a constitutive increase in the amount of (functional) TATPP protein produced. Said increase can also be a temporal decrease in the amount of (functional) TATPP protein produced. An increase in the amount or activity of TATPP can be measured as described elsewhere in this application. An increase in the expression and/or activity of TATPP can be achieved for example by operably linking an TATPP coding region to a promoter, such as any of the promoters described herein below, thereby driving TATPP expression in e.g. a constitutive, inducible, temporal or tissue specific fashion depending on the choice of promoter.

In one embodiment, the nucleic acid encodes a zinc finger protein that binds to the gene encoding an TATPP protein present in the plant, resulting in an increased expression of the target gene. In particular embodiments, the zinc finger protein binds to a regulatory region of said gene, thereby activating its expression. Methods of selecting sites for targeting by zinc finger proteins have been described, for example, in U.S. Pat. No. 6,453,242, and methods for using zinc finger proteins to inhibit the expression of genes in plants are described, for example, in US2003/0037355, each of which is herein incorporated by reference.

In another embodiment, the nucleic acid encodes a TALE protein that binds to a gene encoding an TATPP protein present in the plant, resulting in an increased expression of the gene. In particular embodiments, the TALE protein binds to a regulatory region of said gene, thereby activating its expression. In other embodiments, the TALE protein binds to a messenger RNA encoding said protein and prevents its translation. Methods of selecting sites for targeting by TALE proteins have been described in e.g. Moscou M J, Bogdanove A J (2009) (A simple cipher governs DNA recognition by TAL effectors. Science 326:1501) and Morbitzer R, Romer P, Boch J, Lahaye T (2010) (Regulation of selected genome loci using de novo-engineered transcription activator-like effector (TALE)-type transcription factors. Proc Natl Acad Sci USA 107:21617-21622).

In again a further embodiment, said nucleic acid encodes an TATPP protein, such as an TATPP protein as described elsewhere in this application.

As used herein, the term “plant-expressible promoter” means a DNA sequence that is capable of controlling (initiating) transcription in a plant cell. This includes any promoter of plant origin, but also any promoter of non-plant origin which is capable of directing transcription in a plant cell, i.e., certain promoters of viral or bacterial origin such as the CaMV35S (Harpster et al. (1988) Mol Gen Genet. 212(1):182-90, the subterranean clover virus promoter No 4 or No 7 (WO9606932), or T-DNA gene promoters but also tissue-specific or organ-specific promoters including but not limited to seed-specific promoters (e.g., WO89/03887), organ-primordia specific promoters (An et al. (1996) Plant Cell 8(1):15-30), stem-specific promoters (Keller et al., (1988) EMBO J. 7(12): 3625-3633), leaf specific promoters (Hudspeth et al. (1989) Plant Mol Biol. 12: 579-589), mesophyl-specific promoters (such as the light-inducible Rubisco promoters), root-specific promoters (Keller et al. (1989) Genes Dev. 3: 1639-1646), tuber-specific promoters (Keil et al. (1989) EMBO J. 8(5): 1323-1330), vascular tissue specific promoters (Peleman et al. (1989) Gene 84: 359-369), stamen-selective promoters (WO 89/10396, WO 92/13956), dehiscence zone specific promoters (WO 97/13865) and the like. “Plant-expressible promoters” can also be inducible promoters, such as temperature-inducible promoters or chemically inducible promoters.

Suitable promoters for the invention are constitutive plant-expressible promoters leading to constitutive expression of the chimeric gene of the invention and thus to e. g. a constitutive increase or decrease in the expression and/or activity of an TATPP gene and/or protein. Constitutive plant-expressible promoters are well known in the art, and include the CaMV35S promoter (Harpster et al. (1988) Mol Gen Genet. 212(1):182-90), Actin promoters, such as, for example, the promoter from the Rice Actin gene (McElroy et al., 1990, Plant Cell 2:163), the promoter of the Cassava Vein Mosaic Virus (Verdaguer et al., 1996 Plant Mol. Biol. 31: 1129), the GOS promoter (de Pater et al., 1992, Plant J. 2:837), the Histone H3 promoter (Chaubet et al., 1986, Plant Mol Biol 6:253), the Agrobacterium tumefaciens Nopaline Synthase (Nos) promoter (Depicker et al., 1982, J. Mol. Appl. Genet. 1: 561), or Ubiquitin promoters, such as, for example, the promoter of the maize Ubiquitin-1 gene (Christensen et al., 1992, Plant Mol. Biol. 18:675).

Other suitable promoters for the invention are inducible promoters, such as inducible promoters (e.g. stress-inducible promoters, drought-inducible promoters, hormone-inducible promoters, chemical-inducible promoters, etc.), tissue-specific promoters, developmentally regulated promoters and the like. A variety of plant gene promoters that regulate gene expression in response to environmental, hormonal, chemical, developmental signals, and in a tissue-active manner can be used for expression of a sequence in plants. Choice of a promoter is based largely on the phenotype of interest and is determined by such factors as tissue (e.g., seed, fruit, root, pollen, vascular tissue, flower, carpel, etc.), inducibility (e.g., in response to wounding, heat, cold, drought, light, pathogens, etc.), timing, developmental stage, and the like.

Examples of promoters that can be used to practice this invention are those that elicit expression in response to stresses, such as the RD29 promoters that are activated in response to drought, low temperature, salt stress, or exposure to ABA (Yamaguchi-Shinozaki et al., 2004, Plant Cell, Vol. 6, 251-264; WO12/101118), but also promoters that are induced in response to heat (e.g., see Ainley et al. (1993) Plant MoI. Biol. 22: 13-23), light (e.g., the pea rbcS-3A promoter, Kuhlemeier et al. (1989) Plant Cell 1: 471-478, and the maize rbcS promoter, Schaffher and Sheen (1991) Plant Cell 3: 997-1012); wounding (e.g., wunl, Siebertz et al. (1989) Plant Cell 1: 961-968); pathogens (such as the PR-I promoter described in Buchel et al. (1999) Plant MoI. Biol. 40: 387-396, and the PDF 1.2 promoter described in Manners et al. (1998) Plant MoI. Biol. 38: 1071-1080), and chemicals such as methyl jasmonate or salicylic acid (e.g., see Gatz (1997) Annu. Rev. Plant Physiol. Plant MoI. Biol. 48: 89-108). In addition, the timing of the expression can be controlled by using promoters such as those acting at senescence (e.g., see Gan and Amasino (1995) Plant Cell 13(4): 935-942); or late seed development (e.g., see Odell et al. (1994) Plant Physiol. 106: 447-458).

Use may also be made of salt-inducible promoters such as the salt-inducible NHX1 promoter of rice landrace Pokkali (PKN) (Jahan et al., 6th International Rice Genetics symposium, 2009, poster abstract P4-37), the salt inducible promoter of the vacuolar H+-pyrophosphatase from Thellungiella halophila (TsVP1) (Sun et al., BMC Plant Biology 2010, 10:90), the salt-inducible promoter of the Citrus sinensis gene encoding phospholipid hydroperoxide isoform gpxl (Avsian-Kretchmer et al., Plant Physiology July 2004 vol. 135, p1685-1696).

In alternative embodiments, tissue-specific and/or developmental stage-specific promoters are used, e.g., promoter that can promote transcription only within a certain time frame of developmental stage within that tissue. See, e.g., Blazquez (1998) Plant Cell 10:791-800, characterizing the Arabidopsis LEAFY gene promoter. See also Cardon (1997) Plant J 12:367-77, describing the transcription factor SPL3, which recognizes a conserved sequence motif in the promoter region of the A. thaliana floral meristem identity gene API; and Mandel (1995) Plant Molecular Biology, Vol. 29, pp 995-1004, describing the meristem promoter eIF4. Tissue specific promoters which are active throughout the life cycle of a particular tissue can be used. In one aspect, the nucleic acids of the invention are operably linked to a promoter active primarily only in cotton fiber cells, in one aspect, the nucleic acids of the invention are operably linked to a promoter active primarily during the stages of cotton fiber cell elongation, e.g., as described by Rinehart (1996) supra. The nucleic acids can be operably linked to the Fb12A gene promoter to be preferentially expressed in cotton fiber cells (Ibid). See also, John (1997) Proc. Natl. Acad. Sci. USA 89:5769-5773; John, et al., U.S. Pat. Nos. 5,608,148 and 5,602,321, describing cotton fiber-specific promoters and methods for the construction of transgenic cotton plants. Root-specific promoters may also be used to express the nucleic acids of the invention. Examples of root-specific promoters include the promoter from the alcohol dehydrogenase gene (DeLisle (1990) Int. Rev. Cytol. 123:39-60) and promoters such as those disclosed in U.S. Pat. Nos. 5,618,988, 5,837,848 and 5,905,186. Other promoters that can be used to express the nucleic acids of the invention include, e.g., ovule-specific, embryo-specific, endosperm-specific, integument-specific, seed coat-specific promoters, or some combination thereof; a leaf-specific promoter (see, e.g., Busk (1997) Plant J. 11:1285 1295, describing a leaf-specific promoter in maize); the ORF 13 promoter from Agrobacterium rhizogenes (which exhibits high activity in roots, see, e.g., Hansen (1997) supra); a maize pollen specific promoter (see, e.g., Guerrero (1990) MoI. Gen. Genet. 224:161 168); a tomato promoter active during fruit ripening, senescence and abscission of leaves, a guard-cell preferential promoter e.g. as described in PCT/EP12/065608, and, to a lesser extent, of flowers can be used (see, e.g., Blume (1997) Plant J. 12:731 746); a pistil-specific promoter from the potato SK2 gene (see, e.g., Ficker (1997) Plant MoI. Biol. 35:425 431); the Blec4 gene from pea, which is active in epidermal tissue of vegetative and floral shoot apices of transgenic alfalfa making it a useful tool to target the expression of foreign genes to the epidermal layer of actively growing shoots or fibers; the ovule-specific BEL1 gene (see, e.g., Reiser (1995) Cell 83:735-742, GenBank No. U39944); and/or, the promoter in Klee, U.S. Pat. No. 5,589,583, describing a plant promoter region is capable of conferring high levels of transcription in meristematic tissue and/or rapidly dividing cells. Further tissue specific promoters that may be used according to the invention include: seed-specific promoters (such as the napin, phaseolin or DC3 promoter described in U.S. Pat. No. 5,773,697), fruit-specific promoters that are active during fruit ripening (such as the dru 1 promoter (U.S. Pat. No. 5,783,393), or the 2A1 1 promoter (e.g., see U.S. Pat. No. 4,943,674) and the tomato polygalacturonase promoter (e.g., see Bird et al. (1988) Plant MoI. Biol. 11: 651-662), flower-specific promoters (e.g., see Kaiser et al. (1995) Plant MoI. Biol. 28: 231-243), pollen-active promoters such as PTA29, PTA26 and PTA1 3 (e.g., see U.S. Pat. No. 5,792,929) and as described in e.g. Baerson et al. (1994 Plant MoI. Biol. 26: 1947-1959), promoters active in vascular tissue (e.g., see Ringli and Keller (1998) Plant MoI. Biol. 37: 977-988), carpels (e.g., see OhI et al. (1990) Plant Cell 2:), pollen and ovules (e.g., see Baerson et al. (1993) Plant MoI. Biol. 22: 255-267). In alternative embodiments, plant promoters which are inducible upon exposure to plant hormones, such as auxins, are used to express the nucleic acids used to practice the invention. For example, the invention can use the auxin-response elements El promoter fragment (AuxREs) in the soybean {Glycine max L.) (Liu (1997) Plant Physiol. 115:397-407); the auxin-responsive Arabidopsis GST6 promoter (also responsive to salicylic acid and hydrogen peroxide) (Chen (1996) Plant J. 10: 955-966); the auxin-inducible parC promoter from tobacco (Sakai (1996) 37:906-913); a plant biotin response element (Streit (1997) MoI. Plant Microbe Interact. 10:933-937); and, the promoter responsive to the stress hormone abscisic acid (ABA) (Sheen (1996) Science 274:1900-1902). Further hormone inducible promoters that may be used include auxin-inducible promoters (such as that described in van der Kop et al. (1999) Plant MoI. Biol. 39: 979-990 or Baumann et al., (1999) Plant Cell 11: 323-334), cytokinin-inducible promoter (e.g., see Guevara-Garcia (1998) Plant MoI. Biol. 38: 743-753), promoters responsive to gibberellin (e.g., see Shi et al. (1998) Plant MoI. Biol. 38: 1053-1060, Willmott et al. (1998) Plant Molec. Biol. 38: 817-825) and the like.

In alternative embodiments, nucleic acids used to practice the invention can also be operably linked to plant promoters which are inducible upon exposure to chemical reagents which can be applied to the plant, such as herbicides or antibiotics. For example, the maize In2-2 promoter, activated by benzenesulfonamide herbicide safeners, can be used (De Veylder (1997) Plant Cell Physiol. 38:568-577); application of different herbicide safeners induces distinct gene expression patterns, including expression in the root, hydathodes, and the shoot apical meristem. Coding sequence can be under the control of, e.g., a tetracycline-inducible promoter, e.g., as described with transgenic tobacco plants containing the Avena sativa L. (oat) arginine decarboxylase gene (Masgrau (1997) Plant J. 11:465-473); or, a salicylic acid-responsive element (Stange (1997) Plant J. 11:1315-1324). Using chemically- {e.g., hormone- or pesticide-) induced promoters, i.e., promoter responsive to a chemical which can be applied to the transgenic plant in the field, expression of a polypeptide of the invention can be induced at a particular stage of development of the plant. Use may also be made of the estrogen-inducible expression system as described in U.S. Pat. No. 6,784,340 and Zuo et al. (2000, Plant J. 24: 265-273) to drive the expression of the nucleic acids used to practice the invention.

In alternative embodiments, a promoter may be used whose host range is limited to target plant species, such as corn, rice, barley, wheat, potato or other crops, inducible at any stage of development of the crop.

In alternative embodiments, a tissue-specific plant promoter may drive expression of operably linked sequences in tissues other than the target tissue. In alternative embodiments, a tissue-specific promoter that drives expression preferentially in the target tissue or cell type, but may also lead to some expression in other tissues as well, is used.

According to the invention, use may also be made, in combination with the promoter, of other regulatory sequences, which are located between the promoter and the coding sequence, such as transcription activators (“enhancers”), for instance the translation activator of the tobacco mosaic virus (TMV) described in Application WO 87/07644, or of the tobacco etch virus (TEV) described by Carrington & Freed 1990, J. Virol. 64: 1590-1597, for example.

Other regulatory sequences that enhance the expression of the nucleic acid of the invention may also be located within the chimeric gene. One example of such regulatory sequences are introns. Introns are intervening sequences present in the pre-mRNA but absent in the mature RNA following excision by a precise splicing mechanism. The ability of natural introns to enhance gene expression, a process referred to as intron-mediated enhancement (IME), has been known in various organisms, including mammals, insects, nematodes and plants (WO 07/098042, p11-12). IME is generally described as a posttranscriptional mechanism leading to increased gene expression by stabilization of the transcript. The intron is required to be positioned between the promoter and the coding sequence in the normal orientation. However, some introns have also been described to affect translation, to function as promoters or as position and orientation independent transcriptional enhancers (Chaubet-Gigot et al., 2001, Plant Mol Biol. 45(1):17-30, p2′7-28).

In connection with the present invention suitable examples of genes containing such introns include the 5′ introns from the rice actin 1 gene (see U.S. Pat. No. 5,641,876), the rice actin 2 gene, the maize sucrose synthase gene (Clancy and Hannah, 2002, Plant Physiol. 130(2):918-29), the maize alcohol dehydrogenase-1 (Adh-1) and Bronze-1 genes (Callis et al. 1987 Genes Dev. 1(10):1183-200; Mascarenhas et al. 1990, Plant Mol Biol. 15(6):913-20), the maize heat shock protein 70 gene (see U.S. Pat. No. 5,593,874), the maize shrunken 1 gene, the light sensitive 1 gene of Solanum tuberosum, and the heat shock protein 70 gene of Petunia hybrida (see U.S. Pat. No. 5,659,122), the replacement histone H3 gene from alfalfa (Keleman et al. 2002 Transgenic Res. 11(1):69-72) and either replacement histone H3 (histone H3.3-like) gene of Arabidopsis thaliana (Chaubet-Gigot et al., 2001, Plant Mol Biol. 45(1):17-30).

Other suitable regulatory sequences include 5′ UTRs. As used herein, a 5′UTR, also referred to as leader sequence, is a particular region of a messenger RNA (mRNA) located between the transcription start site and the start codon of the coding region. It is involved in mRNA stability and translation efficiency. For example, the 5′ untranslated leader of a petunia chlorophyll a/b binding protein gene downstream of the 35S transcription start site can be utilized to augment steady-state levels of reporter gene expression (Harpster et al., 1988, Mol Gen Genet. 212(1):182-90). WO95/006742 describes the use of 5′ non-translated leader sequences derived from genes coding for heat shock proteins to increase transgene expression. A “3′ end region involved in transcription termination and polyadenylation functional in plants” as used herein is a sequence that drives the cleavage of the nascent RNA, whereafter a poly(A) tail is added at the resulting RNA 3′ end, functional in plant cells. Transcription termination and polyadenylation signals functional in plant cells include, but are not limited to, 3′nos, 3′35S, 3′his and 3′g7.

“Introducing” in this respect, relates to the placing of genetic information in a plant cell or plant by artificial means, such as transformation. This can be effected by any method known in the art for introducing RNA or DNA into plant cells, tissues, protoplasts or whole plants. In addition to artificial introduction as described above, “introducing” also comprises introgressing genes as defined further below.

Transformation means introducing a nucleotide sequence into a plant in a manner to cause stable or transient expression of the sequence. Transformation and regeneration of both monocotyledonous and dicotyledonous plant cells is now routine, and the selection of the most appropriate transformation technique will be determined by the practitioner. The choice of method will vary with the type of plant to be transformed; those skilled in the art will recognize the suitability of particular methods for given plant types. Suitable methods can include, but are not limited to: electroporation of plant protoplasts; liposome-mediated transformation; polyethylene glycol (PEG) mediated transformation; transformation using viruses; micro-injection of plant cells; micro-projectile bombardment of plant cells; vacuum infiltration; and Agrobacterium-mediated transformation.

In alternative embodiments, the invention uses Agrobacterium tumefaciens mediated transformation. Also other bacteria capable of transferring nucleic acid molecules into plant cells may be used, such as certain soil bacteria of the order of the Rhizobiales, e.g. Rhizobiaceae (e.g. Rhizobium spp., Sinorhizobium spp., Agrobacterium spp); Phyllobacteriaceae (e.g. Mesorhizobium spp., Phyllobacterium spp.); Brucellaceae (e.g. Ochrobactrum spp.); Bradyrhizobiaceae (e.g. Bradyrhizobium spp.), and Xanthobacteraceae (e.g. Azorhizobium spp.), Agrobacterium spp., Rhizobium spp., Sinorhizobium spp., Mesorhizobium spp., Phyllobacterium spp. Ochrobactrum spp. and Bradyrhizobium spp., examples of which include Ochrobactrum sp., Rhizobium sp., Mesorhizobium loti, Sinorhizobium meliloti. Examples of Rhizobia include R. leguminosarum by, trifolii, R. leguminosarum bv, phaseoli and Rhizobium leguminosarum, by, viciae (U.S. Pat. No. 7,888,552). Other bacteria that can be employed to carry out the invention which are capable of transforming plants cells and induce the incorporation of foreign DNA into the plant genome are bacteria of the genera Azobacter (aerobic), Closterium (strictly anaerobic), Klebsiella (optionally aerobic), and Rhodospirillum (anaerobic, photosynthetically active). Transfer of a Ti plasmid was also found to confer tumor inducing ability on several Rhizobiaceae members such as Rhizobium trifolii, Rhizobium leguminosarum and Phyllobacterium myrsinacearum, while Rhizobium sp. NGR234, Sinorhizobium meliloti and Mesorhizobium loti could indeed be modified to mediate gene transfer to a number of diverse plants (Broothaerts et al., 2005, Nature, 433:629-633).

In alternative embodiments, making transgenic plants or seeds comprises incorporating sequences used to practice the invention and, in one aspect (optionally), marker genes into a target expression construct (e.g., a plasmid), along with positioning of the promoter and the terminator sequences. This can involve transferring the modified gene into the plant through a suitable method. For example, a construct may be introduced directly into the genomic DNA of the plant cell using techniques such as electroporation and microinjection of plant cell protoplasts, or the constructs can be introduced directly to plant tissue using ballistic methods, such as DNA particle bombardment. For example, see, e.g., Christou (1997) Plant MoI. Biol. 35:197-203; Pawlowski (1996) MoI. Biotechnol. 6:17-30; Klein (1987) Nature 327:70-73; Takumi (1997) Genes Genet. Syst. 72:63-69, discussing use of particle bombardment to introduce transgenes into wheat; and Adam (1997) supra, for use of particle bombardment to introduce YACs into plant cells. For example, Rinehart (1997) supra, used particle bombardment to generate transgenic cotton plants. Apparatus for accelerating particles is described U.S. Pat. No. 5,015,580; and, the commercially available BioRad (Biolistics) PDS-2000 particle acceleration instrument; see also, John, U.S. Pat. No. 5,608,148; and Ellis, U.S. Pat. No. 5,681,730, describing particle-mediated transformation of gymnosperms.

In alternative embodiments, protoplasts can be immobilized and injected with a nucleic acids, e.g., an expression construct. Although plant regeneration from protoplasts is not easy with cereals, plant regeneration is possible in legumes using somatic embryogenesis from protoplast derived callus. Organized tissues can be transformed with naked DNA using gene gun technique, where DNA is coated on tungsten microprojectiles, shot 1/100th the size of cells, which carry the DNA deep into cells and organelles. Transformed tissue is then induced to regenerate, usually by somatic embryogenesis. This technique has been successful in several cereal species including maize and rice.

In alternative embodiments, a third step can involve selection and regeneration of whole plants capable of transmitting the incorporated target gene to the next generation. Such regeneration techniques rely on manipulation of certain phytohormones in a tissue culture growth medium, typically relying on a biocide and/or herbicide marker that has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee (1987) Ann. Rev. of Plant Phys. 38:467-486. To obtain whole plants from transgenic tissues such as immature embryos, they can be grown under controlled environmental conditions in a series of media containing nutrients and hormones, a process known as tissue culture. Once whole plants are generated and produce seed, evaluation of the progeny begins.

Viral transformation (transduction) may also be used for transient or stable expression of a gene, depending on the nature of the virus genome. The desired genetic material is packaged into a suitable plant virus and the modified virus is allowed to infect the plant. The progeny of the infected plants is virus free and also free of the inserted gene. Suitable methods for viral transformation are described or further detailed e. g. in WO 90/12107, WO 03/052108 or WO 2005/098004.

In alternative embodiments, after the chimeric gene is stably incorporated in transgenic plants, it can be introduced into other plants by sexual crossing or introgression. Any of a number of standard breeding techniques can be used, depending upon the species to be crossed. Since transgenic expression of the nucleic acids of the invention leads to phenotypic changes, plants comprising the recombinant nucleic acids of the invention can be sexually crossed with a second plant to obtain a final product. Thus, the seed of the invention can be derived from a cross between two transgenic plants of the invention, or a cross between a plant of the invention and another plant. The desired effects (e.g., expression of the polypeptides of the invention to produce a plant in which flowering behavior is altered) can be enhanced when both parental plants express the polypeptides, e.g., an TaTPP gene of the invention. The desired effects can be passed to future plant generations by standard propagation means.

Successful examples of the modification of plant characteristics by transformation with cloned sequences which serve to illustrate the current knowledge in this field of technology, and include for example: U.S. Pat. Nos. 5,571,706; 5,677,175; 5,510,471; 5,750,386; 5,597,945; 5,589,615; 5,750,871; 5,268,526; 5,780,708; 5,538,880; 5,773,269; 5,736,369 and 5,619,042.

In some embodiments, following transformation, plants are selected using a dominant selectable marker incorporated into the transformation vector. Such a marker can confer antibiotic or herbicide resistance on the transformed plants, and selection of transformants can be accomplished by exposing the plants to appropriate concentrations of the antibiotic or herbicide.

In some embodiments, after transformed plants are selected and grown to maturity, those plants showing a modified trait are identified. The modified trait can be any of those traits described above. In alternative embodiments, to confirm that the modified trait is due to changes in expression levels or activity of the transgenic polypeptide or nucleic acid can be determined by analyzing mRNA expression using Northern blots, RT-PCR or microarrays, or protein expression using immunoblots or Western blots or gel shift assays.

“Introgressing” means the integration of a gene in a plant's genome by natural means, i.e. by crossing a plant comprising the chimeric gene or mutant allele described herein with a plant not comprising said chimeric gene or mutant allele. The offspring can be selected for those comprising the chimeric gene or mutant allele.

Cereal plants, also called grain plants, include, but are not limited to, Rice (Oryza sativa), Wheat (Triticum aestivum) Durum wheat, macaroni wheat (Triticum durum), Corn or maize (Zea mays), Job's Tears, salay, tigbe, pawas (Coix lachryma-jobi), Barley (Hordeum vulgare), Millet (Panicum miliaceum, Eleusine coracana, Setaria italica, Pennisetum glaucum), Sorghum (Sorghum bicolor), Oat (Avena sativa), Rye (Secale cereale), Triticale (xTriticosecale), Teff, taf or khak shir (Eragrostis tef), Fonio (Digitaria exilis), Wild rice, Canada rice, Indian rice, water oats (Zizania spp.), Spelt (Triticum spelta), Canary grass (Phalaris sp.).

Wheat plants as used herein are plants of the Triticum ssp, such as Triticum aestivum and Triticum durum or Triticum spelta

As used herein, at least 80% sequence identity can be at least 80%, at least 85%, at least 90%, at least 95%, at least 98%, at least 99% or 100% sequence identity.

A nucleic acid or polynucleotide, as used herein, can be DNA or RNA, single- or double-stranded. Nucleic acids can be synthesized chemically or produced by biological expression in vitro or even in vivo. Nucleic acids can be chemically synthesized using appropriately protected ribonucleoside phosphoramidites and a conventional DNA/RNA synthesizer. Suppliers of RNA synthesis reagents are for example Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). In connection with the chimeric gene of the present disclosure, DNA includes cDNA and genomic DNA.

The terms “protein” or “polypeptide” as used herein describe a group of molecules consisting of more than 30 amino acids, whereas the term “peptide” describes molecules consisting of up to 30 amino acids. Proteins and peptides may further form dimers, trimers and higher oligomers, i.e. consisting of more than one (poly)peptide molecule. Protein or peptide molecules forming such dimers, trimers etc. may be identical or non-identical. The corresponding higher order structures are, consequently, termed homo- or heterodimers, homo- or heterotrimers etc. The terms “protein” and “peptide” also refer to naturally modified proteins or peptides wherein the modification is effected e.g. by glycosylation, acetylation, phosphorylation and the like. Such modifications are well known in the art.

The term “comprising” is to be interpreted as specifying the presence of the stated parts, steps or components, but does not exclude the presence of one or more additional parts, steps or components. A plant comprising a certain trait may thus comprise additional traits.

It is understood that when referring to a word in the singular (e.g. plant or root), the plural is also included herein (e.g. a plurality of plants, a plurality of roots). Thus, reference to an element by the indefinite article “a” or “an” does not exclude the possibility that more than one of the element is present, unless the context clearly requires that there be one and only one of the elements. The indefinite article “a” or “an” thus usually means “at least one”.

For the purpose of this invention, the “sequence identity” of two related nucleotide or amino acid sequences, expressed as a percentage, refers to the number of positions in the two optimally aligned sequences which have identical residues (×100) divided by the number of positions compared. A gap, i.e., a position in an alignment where a residue is present in one sequence but not in the other, is regarded as a position with non-identical residues. The “optimal alignment” of two sequences is found by aligning the two sequences over the entire length according to the Needleman and Wunsch global alignment algorithm (Needleman and Wunsch, 1970, J Mol Biol 48(3):443-53) in The European Molecular Biology Open Software Suite (EMBOSS, Rice et al., 2000, Trends in Genetics 16(6): 276-277; see e.g. http://www.ebi.ac.uk/emboss/align/index.html) using default settings (gap opening penalty=10 (for nucleotides)/10 (for proteins) and gap extension penalty=0.5 (for nucleotides)/0.5 (for proteins)). For nucleotides the default scoring matrix used is EDNAFULL and for proteins the default scoring matrix is EBLOSUM62.

“Substantially identical” or “essentially similar”, as used herein, refers to sequences, which, when optimally aligned as defined above, share at least a certain minimal percentage of sequence identity (as defined above further below).

Whenever reference to a “plant” or “plants” according to the invention is made, it is understood that also plant parts cells, tissues or organs, seed pods, seeds, severed parts such as roots, leaves, flowers, pollen, etc. are included. Whenever reference to a “plant” or “plants” according to the invention is made, it is understood that also progeny of the plants which retain the distinguishing characteristics of the parents (especially modulated flowering time, seed development, seed maturation or modulated seed germination), such as seed obtained by selfing or crossing, e.g. hybrid seed (obtained by crossing two inbred parental lines), hybrid plants and plant parts derived there from are encompassed herein, such as progeny comprising a chimeric gene or mutant/knock-out TATPP allele according to the invention, unless otherwise indicated.

Creating propagating material”, as used herein, relates to any means know in the art to produce further plants, plant parts or seeds and includes inter alia vegetative reproduction methods (e.g. air or ground layering, division, (bud) grafting, micropropagation, stolons or runners, storage organs such as bulbs, corms, tubers and rhizomes, striking or cutting, twin-scaling), sexual reproduction (crossing with another plant) and asexual reproduction (e.g. apomixis, somatic hybridization).

Unless stated otherwise in the Examples, all recombinant DNA techniques are carried out according to standard protocols as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Croy, jointly published by BIOS Scientific Publications Ltd (UK) and Blackwell Scientific Publications, UK. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

All patents, patent applications, and publications or public disclosures (including publications on internet) referred to or cited herein are incorporated by reference in their entirety.

The work underlying the present invention has been supported by the project “Molecular Basis of Formation of Main Crop Yield Traits” (Project lot number: 2016YFD0100402, Task Leader: Hongxia LIU) in the 13^{th} Five-Year Plan by the Ministry of Science and Technology and by the National Natural Fund “Functional Analysis of Important Candidate Genes Associated with Wheat 5DS Grain Yield and Study on the Regulatory Mechanism Thereof” (Project lot number: 31471492; Project Leader: Hongxia LIU)”.

Throughout the specification reference is made to the following entries in the Sequence Listing:

SEQ ID No. 1: amino acid sequence of TaTPP-7A

SEQ ID No. 2: nucleotide sequence of the coding region (cDNA) for TaTPP-7A

SEQ ID No. 3: nucleotide sequence of the genomic region (gDNA) for TaTPP-7A

SEQ ID No. 4: forward primer TaTPP-F1

SEQ ID No. 5: reverse primer TaTPP-R1

SEQ ID No. 6: forward primer TaTPPcDNA-F1

SEQ ID No. 7: reverse primer TaTPPcDNA-R1

SEQ ID No. 8: forward primer QST-TPP-7A-F

SEQ ID No. 9: reverse primer QST-TPP-7A-R

SEQ ID No. 10: forward primer (cloning) TPP-TaA-F

SEQ ID No. 11: reverse primer (cloning) TPP-TaA-R

SEQ ID No 12: forward primer TPP-P-1F (promoter amplification)

SEQ ID No 13: reverse primer TPP-P-1R (promoter amplification)

SEQ ID No 14: TPP-7A promoter version 1

SEQ ID No 15: TPP-7A promoter version 2

SEQ ID No 16: forward primer TPP-P-TF

SEQ ID No 17: reverse primer TPP-P-TR

SEQ ID No 18: nucleotide sequence between SNP 493 and SNP 1980 as in SEQ ID No. 14

SEQ ID No 19: nucleotide sequence between positions 467-514 of the 5′ end of PCR amplification TaTPP version 1

SEQ ID No. 20: nucleotide sequence between positions 467-514 of the 5′ end of PCR amplification TaTPP version 2

SEQ ID No. 21: nucleotide sequence of KASP based primer 488F1

SEQ ID No. 22: nucleotide sequence of KASP based primer 488F2

SEQ ID No. 23: nucleotide sequence of KASP primer 488C

SEQ ID No. 24: nucleotide sequence of SNP 488 marker

SEQ ID No 25: nucleotide sequence between positions 2121-2168 of the 5′ end of PCR amplification TaTPP version 1

SEQ ID No. 26: nucleotide sequence between positions 2121-2168 of the 5′ end of PCR amplification TaTPP version 2

SEQ ID No. 27: nucleotide sequence of KASP based primer 2144F1

SEQ ID No. 28: nucleotide sequence of KASP based primer 2144F2

SEQ ID No. 29: nucleotide sequence of KASP primer 2144C

SEQ ID No. 30: nucleotide sequence of SNP 2144C marker

### EXAMPLES

The following examples are provided to facilitate a better understanding of the present invention, but are not intended to limit the invention. The experimental methods in the following examples are conventional methods, unless otherwise specified. The test materials used in the following examples are commercially available from conventional biochemical reagent stores, unless otherwise specified. In the following examples, each quantitative test is repeated thrice, and the results are averaged.

Vector PCambia3301: YouBio, product number VT1386.

Vector PWMB003: Maoyun YU, Guixiang YIN, Pingzhi ZHANG Xingguo YE, Construction and Validation of Three Vectors for Genetic Transformation of Crops, 2014 Annual Conference: Transgenic Crop Research and Safety Management, 58-67.

Agrobacterium tumefaciens GV3101: Reference literature: Yadav S, Sharma P, Srivastava A, Desai P, Shrivastava N. Strain specific Agrobacterium-mediated genetic transformation of Bacopa monnieri. Journal of Genetic Engineering and Biotechnology. 2014, 12:89-94.

Wheat Fielder: Reference literature: Richardson T, Thistleton J, Higgins T J, Howitt C, Ayliffe M. Efficient Agrobacterium transformation of elite wheat germplasm without selection. Plant Cell Tiss Organ Cult. 2014, DOI 10.1007/s11240-014-0564-7.

### Example 1. Cloning of Protein TaTPP-7A and Coding Gene Thereof

According to the kernel weight correlation analysis in a wheat natural population (239 wheat lines), the fine localization analysis of SSR molecular markers in a mapping population (wheat kernel weight F2 segregating population), the genomic sequence information of candidate genes obtained by BAC library screening and comparative genomic approaches in the early stage in the lab, primers were designed to amplify the target TPP genes from the diploid ancestor A genomic wheat (Triticum urartu) and common hexaploid wheat (Chinese Spring Wheat), respectively.

The genomic DNA of Triticum urartu was extracted, subjected to PCR amplification with a primer pair composed of TaTPP-F1 and TaTPP-R1. The PCR amplification products were subjected to TA cloning sequencing, and 15 positive clones were selected for sequencing.

The genomic DNA of Chinese Spring Wheat was extracted, subjected to a first cycle of PCR amplification with a primer pair composed of TaTPP-F1 and TaTPP-R1, and then to a second cycle of PCR amplification with a primer pair composed of TaTPP-F1cDNA-F1 and TaTPP-R1cDNA-R1, using the amplification product of the first cycle as template. The PCR amplification products were subjected to TA cloning sequencing, and 15 positive clones were selected for sequencing.

The sequencing results showed that the corresponding PCR amplification product of Triticum urartu was as shown by SEQ ID NO:3 in Sequence Listing, and the product of second cycle of PCR amplification corresponding to Chinese Spring Wheat was as shown by the nucleotides at positions 23-2115 from 5′ terminal of SEQ ID NO:3 in Sequence Listing.

The protein as shown by SEQ ID NO:1 in Sequence Listing was designated as protein TaTPP-7A. The gene encoding the protein TaTPP-7A was designated as gene TaTPP-7A, whose genomic sequence was as shown by SEQ ID NO:3 in Sequence Listing, and cDNA sequence was as shown by SEQ ID NO:2 in Sequence Listing.

Specific subgenomic locating primers (QST-TPP-7A-F and QST-TPP-7A-R) were designed by alignment analysis, the above sequences were further subjected to chromosomal localization analysis using the nullisomic-tetrasomic material from 7^{th} homologous group of wheat to locate the gene TaTPP-7A on the wheat chromosome 7A, and further finely locate the gene TaTPP-7A on wheat 7As.

TaTPP-F1:
(SEQ ID NO: 4)
5′-CGTGTGGTTGTTTGCGTG-3′;
TaTPP-R1:
(SEQ ID NO: 5)
5′-CTAGATATAGGCGAGGGTTATTAC-3′;
TaPP1cDNA-F1:
(SEQ ID NO: 6)
5′-ATGGCGAACCAGGACGT-3′;
TaPP1cDNA-R1:
(SEQ ID NO: 7)
5′-CTACACTCTTGCGCGCAT-3′;
QST-TPP-TA-F:
(SEQ ID NO: 8)
5′-CCATGCCTTGTCCTTGATGT-3′;
QST-TPP-TA-R:
(SEQ ID NO: 9)
5′-AAACCAAGAAAAGCGAGAGATC-3′.

### Example 2. Production and Identification of Transgenic Wheat Plants Overexpressing TaTPP

I. Construction of Recombinant Plasmids

1. A double-stranded DNA molecule comprising the nucleotide sequence of SEQ ID NO: 2 in Sequence Listing was synthesized.

2. Using the DNA molecule synthesized from step 1 as template, a primer set composed of TPP-TaA-F and TPP-TaA-R was used for PCR amplification.

TPP-TaA-F:
(SEQ ID NO: 10)
5′-CGGGATCCATGGCGAACCAGGACGT-3′
TPP-TaA-R:
(SEQ ID NO: 11)
5′-CGGAATTCCTACACTCTTGCGCGCAT-3′.

3. The PCR amplification product obtained from step 2 was subjected to a double enzyme cut by using the restriction endonucleases Bam HI and Eco RI, and the enzyme cutting product was recovered.

4. Construction of Recombinant Plasmid pWMB110

(1) The vector pCambia3301 was selected, subjected to a double enzyme cut by using the restriction endonucleases EcoRI and Pm11, and the vector backbone (about 8.5 kb) was recovered.

(2) The vector pWMB003 was selected, subjected to a double enzyme cut by using the restriction endonucleases HindIII and EocRI, and about 2.2 kb of Ubi-MCS-Nos fragment was recovered.

(3) The vector backbone obtained from step (1) and the Ubi-MCS-Nos fragment obtained from step (2) were connected via In-Fusion HD Cloning Kit (a product from Company Takara), resulting in the recombinant plasmid pWMB110.5.

5. The recombinant plasmid pWMB110 was selected and subject to a double enzyme cut by using the restriction endonucleases Bam HI and Eco RI, and a vector backbone of about 10.6 kb was recovered.

6. The enzyme cutting product from step 3 and the vector backbone from step 5 were connected to give a recombinant plasmid pWMB110-TaTPP-7A. According to the sequencing results, the structure of recombinant plasmid pWMB110-TaTPP-7A was described as follows: the small fragment between the Bam HI and Eco RI enzyme cutting sites was as shown by SEQ ID NO:2 in Sequence Listing.

II. Production of Transgenic Plants

1. The recombinant plasmid pWMB110-TaTPP-7A was introduced into Agrobacterium tumefaciens GV3101 to obtain a recombinant Agrobacterium.

2. The recombinant Agrobacterium obtained from step 1 was used for genetic transformation of the immature embryo callus of wheat Fielder and then cultivated to obtain T₀ regenerated plants. The T₀ regenerated plants were self-bred to give T₁ generation plants. The T₁ generation plants were self-bred to obtain T₂ generation plants.

The T₀ regenerated plants, T₁ generation plants and T₂ generation plants were subjected to “Bar gene” identification and target gene identification. The specific steps were as follows: The leaves of the plants were first taken and subjected to gene Bar identification using Envirologix® PAT/bar transgenic kit operated according to the instructions; the plants shown to be positive according to gene Bar identification was further subjected to target gene identification (the genomic DNA of leaves was extracted and subjected to PCR identification using a primer pair composed of TPP-TaA-F and TPP-TaA-R, and if 1.1 kb of amplification product was obtained, then the plants were considered positive according to PCR identification). If the identification was positive for particular T₀ and T₁ generation plants at a plant separation ratio of 3:1, and the T₂ generation plant is positive according to PCR identification and no segregation of traits occurs in the progeny, then the T₂ and its self-bred progeny is considered to a homozygous transgenic line.

Three homozygous transgenic lines (TaTPP-5-3 line, TaTPP-10-4 line and TaTPP-13-7 line) were randomly selected for trait identification.

III. Production of Control Plants Transformed with an Empty Vector

The recombinant plasmid pWMB110 was used in place of the recombinant plasmid pWMB110-TaTPP-7A, to transform wheat plants as described in section II, giving a control line transformed with an empty vector.

IV. Trait Identification

The tested transgenic lines were: T₂ generation plants of TaTPP-5-3 line, T₂ generation plants of TaTPP-10-4 line, T₂ generation plants of TaTPP-13-7 cell, T₂ generation plant line transformed with empty vector and wheat Fielder as control plants.

Each line consisted of 50 plants.

Each test line was cultured in parallel (i.e., cultivated in the same land and cultured under exactly the same conditions), and grains were harvested at harvest time. The average kernel length, average kernel width, average kernel thickness and average thousand-kernel weight of grains in each line were measured.

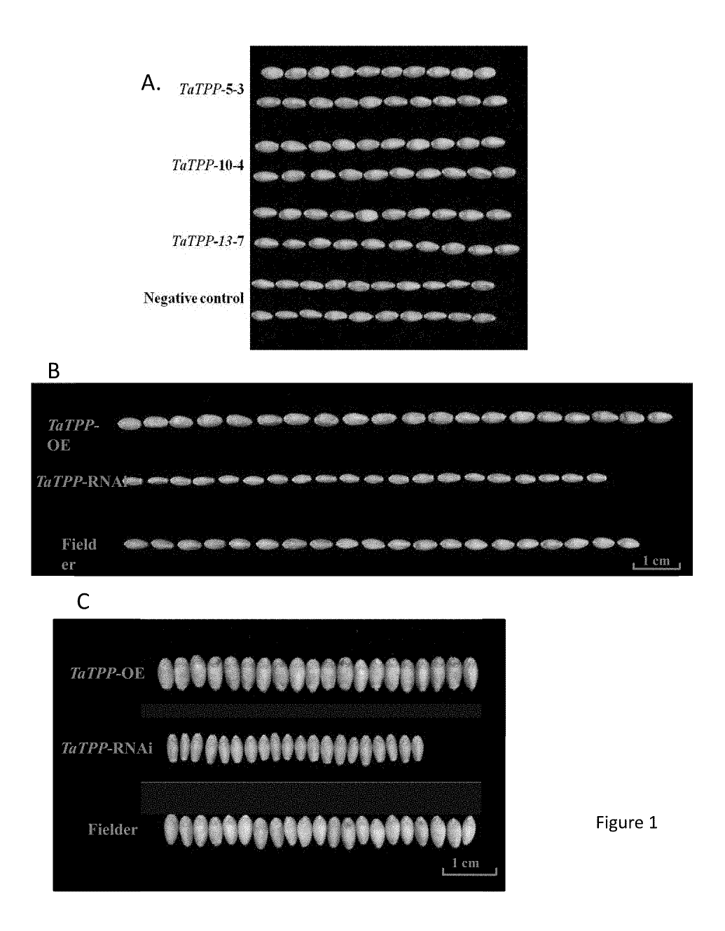

FIG. 1 shows photographs of grains from transgenic wheat lines overexpressing TaTPP as compared to untransformed control plants (Fielder) and transformed control plants wherein the expression of TaTPP was reduced. The phenotype of grains from TaTPP-10-4 line, and the phenotype of grains from TaTPP-13-7 line did not exhibit any significant difference from the phenotype of grains from TaTPP-5-3 line in FIG. 1. The phenotype of grains from the line transformed with empty vector control plants did not exhibit any significant difference from the phenotype of grains from untransformed control wheat Fielder in FIG. 1. Grains from TaTPP overexpressing wheat lines did show an increase in grain length, thousand kernel weight and grain width relative to the control plants.

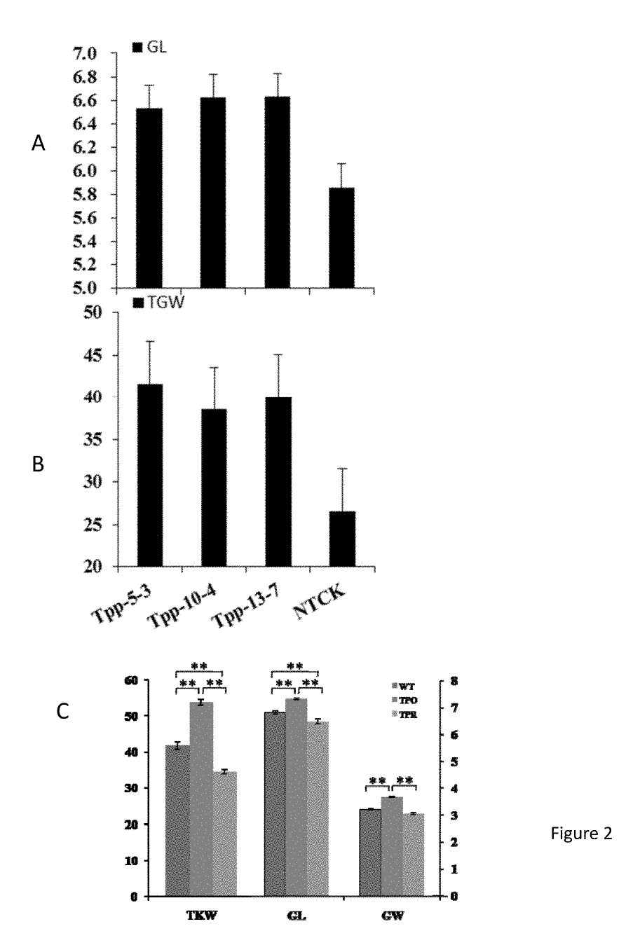

FIG. 2 shows the measurements for grain length, thousand kernel weight for from transgenic wheat lines overexpressing TaTPP as compared to untransformed control plants (Fielder) and transformed control plants wherein the expression of TaTPP was reduced.

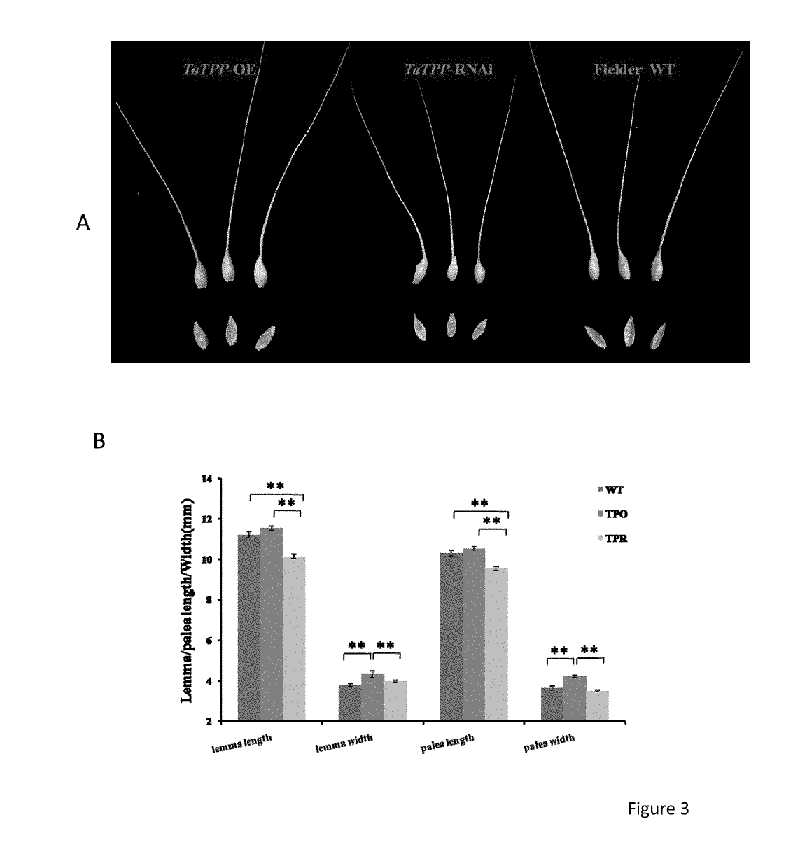

FIG. 3 shows measurements and photographs demonstrating that transgenic plants overexpressing Ta TPP had increased lemma length, width, palea length and palea width.

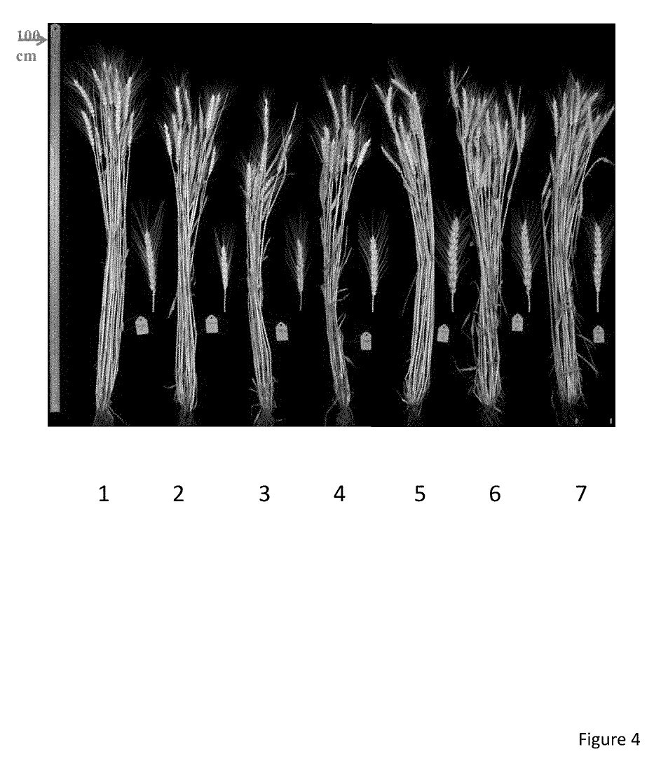

FIG. 4 shows photographs of the increased tiller length, and spike length in transgenic plants overexpressing TaTPP as compared to untransformed control plants (Fielder) and transformed control plants wherein the expression of TaTPP was reduced.

The average kernel length, average kernel width, average kernel thickness and average thousand-kernel weight of grains in each line were as shown in Table 1. Some results were as shown in FIG. 2. The kernel length, kernel width and kernel thickness of grains in each transgenic line were all higher than those in wheat Fielder, showing significant differences. The kernel length, kernel width and kernel thickness of grains in the line transformed with empty vector were essentially consistent with those in wheat Fielder. The average thousand-kernel weight of three transgenic lines was 41.6 g, 38.53 g and 40.1 g, respectively, which had been greatly improved compared to wheat Fielder (26.5 g), showing a remarkably significant difference (P<0.001). The results showed that protein TaTPP-7A had a positive regulatory effect on wheat yield, and was capable of increasing thousand-kernel weight and kernel length.

TABLE 1

| | | | | | Line transformed |
|---|---|---|---|---|---|
| | TaTPP-5-3 | TaTPP-10-4 | TaTPP-13-7 | Fielder | with empty vector |
| Average kernel | 6.53 | 6.568 | 6.625 | 5.863 | 5.658 |
| length (cm) | | | | | |
| Average kernel | 3.40 | 3.33 | 3.495 | 2.884 | 2.879 |
| width (cm) | | | | | |
| Average kernel | 3.10 | 3.06 | 3.0575 | 2.483 | 2.469 |
| thickness (cm) | | | | | |
| Average | 41.6 | 38.53 | 40.1 | 26.5 | 26.3 |
| thousand-kernel | | | | | |
| weight (g) | | | | | |

### Example 3 Production and Identification of Transgenic Arabidopsis Plants Overexpressing TaTPP

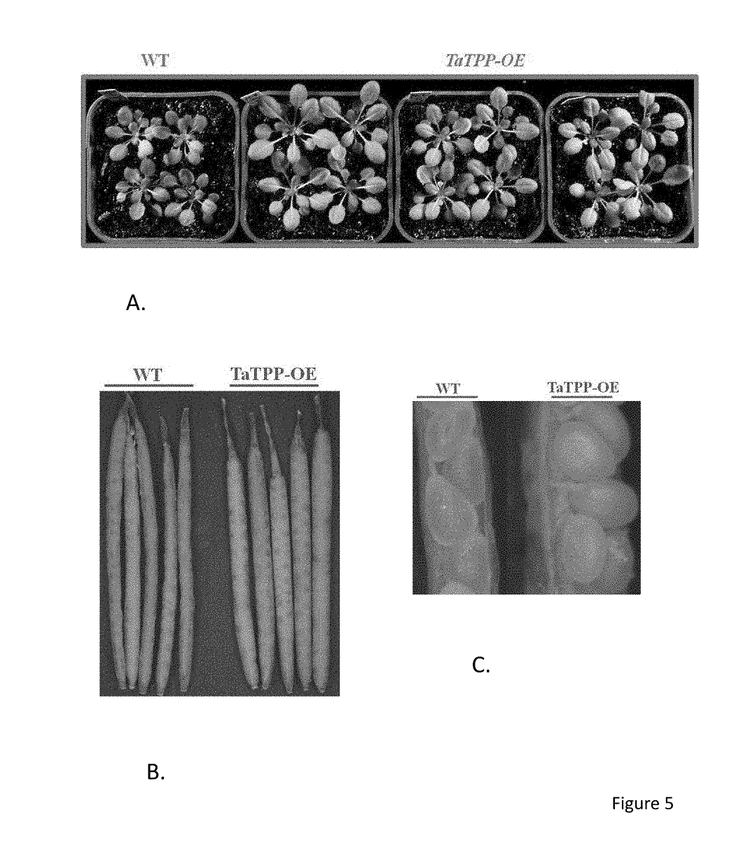

Recombinant vectors and Agrobacteria as described in Example 2 were also used to generate transgenic Arabidopsis plants overexpressing TaTPP. As shown in FIG. 5, these transgenic plants exhibited an increased biomass production of vegetative growth, altered pod morphology and increased seed size when compared to untransformed Arabidopsis control plants.

### Example 4. Isolation of Promoter Regions from TaTPP from Various Wheat Varieties

I. Material and Methods

Vector pDONR207: product of Invitrogen Corporation, plasmid map accession number: 02352

pGWB35: BioVector NTCC Liu J, Zhang T R, Jia J Z, Sun J Q. 2016. The wheat mediator subunit TaMED25 interacts with the transcription factor TaEIL1 to negatively regulate disease resistance against Powdery Mildew. Plant Physiology. 170: 1799-1816.

Tobacco used in these examples is Nicotiana benthamiana. References: Agrobacterium-mediated factors influencing transient expression in tobacco; Sun Manli, Meng Yu, Zhang Qiang, Huang Guiyan, Shan Weixing; Northwest China Journal of Agricultural Sciences, 2015, 24 1): 161-165.

The plant imaging system used in the examples was Nightshade LB985, Berthold technologies

II. Isolation of Two Different Types of Promoters for Ta TPP-7A from Wheat.

34 wheat lines with different grain traits (numbered C1-34 see Table 2) were selected as the materials for isolation of the promoter regions for TaTPP-7A.

Each of the test lines were subjected to the following steps:

1. extracting the genomic DNA for the tested wheat line

2. Using the genomic DNA extracted in step 1 as a template, PCR amplification was carried out by using primer pairs consisting of TPP-P-1F and TPP-P-1R to obtain PCR amplification products.

TPP-P-1F (SEQ ID No: 12 of Sequence Listing):
5′-GAATGTAGCAGTCCACCTAT-3′;
TPP-P-1R(SEQ ID No: 13 of the Sequence Listing):
5′-ACGCAGATCAATCATCAGAA-3″.

3 take the PCR amplification product obtained in step 2, clone and sequence. Twenty-five clones per wheat line.

4. Assemble the sequences and compare.

Twenty-five clones of each wheat material were sequenced and analyzed for the A genome promoter sequence of TaTPP. The PCR amplification product consists of two parts, one part is the promoter region (from the 5′ end until the ATG start codon) and the other part is the coding region (from the ATG to the 3′ end) Two versions of the TaTPP-7A promoters were found from 34 wheat cultivars, one shown in SEQ ID No 14 (named P1 promoter) and the other as shown in SEQ ID No 15 (named P2 promoter).

III. Functional Verification of the Promoter Regions

Recombinant Plasmids

1. Double stranded DNA molecule as shown in SEQ ID NO: 14 were synthesized.

2. Using the DNA molecule obtained in step 1 as a template, PCR amplification was carried out by using primer pairs consisting of TPP-P-TF and TPP-P-TR to obtain PCR amplification products. TPP-P-TF, the attB1 sequence is underlined. In TPP-P-TR, the attB2 sequence is underlined.

TPP-P-TF (SEQ ID NO: 16)
5′-GGGGACAAGTTTGTACAAAAAAGCAGGCTTCCTCTTGATAAGTGTC
GGAGGACC-3′;
TPP-P-TR (SEQ ID NO: 17):
5′-GGGGACCACTTTGTACAAGAAAGCTGGGTCGGCGCACGCAAACAAC
C-3′

3. The PCR amplification product obtained in Step 2 was subjected to BP recombination with the vector pDONR207 to obtain a recombinant plasmid having the DNA molecule shown in the 217th to 4997th nucleotides of SEQ ID No:14.

4. The recombinant plasmid obtained in step 3 undergoes an LR reaction with the vector pGWB35 to obtain a recombinant plasmid with the DNA molecule shown by the 217th to 4997th nucleotides of the SEQ ID No:14 operably linked in the forward direction of the pGWB35 vector to the fluorescent gene resulting in Recombinant plasmid-P1. The pGWB35 vector has a fluorescent gene, and the DNA molecule shown by the 217th to the 497th nucleotides of SEQ ID No: 14 is inserted in front of the fluorescent gene to verify its promoter activity.

5. Double stranded DNA molecules shown in SEQ ID NO: 15 are synthesized.

6. Using the DNA molecule obtained in step 5 as a template, PCR amplification was carried out by using primer pairs consisting of TPP-P-TF and TPP-P-TR to obtain PCR amplification products.

7. The PCR amplification product obtained in Step 6 was subjected to BP recombination with the vector pDONR207 to obtain a recombinant plasmid having the DNA molecule shown by the nucleotide numbers 217-2498 of SEQ ID NO: 15.

8. The recombinant plasmid obtained in step 7 undergoes an LR reaction with the vector pGWB35 to obtain a recombinant plasmid with the DNA molecule shown by the 217th to 4997th nucleotides of the SEQ ID No:15 operably linked in the forward direction of the pGWB35 vector to the fluorescent gene resulting in Recombinant plasmid-P2. The pGWB35 vector has a fluorescent gene, and the DNA molecule shown by the 217th to the 497th nucleotides of SEQ ID No: 15 is inserted in front of the fluorescent gene to verify its promoter activity.

Functional Verification of the Promoter Regions

The tested plasmids were: recombinant plasmid-P1 or recombinant plasmid-P2 or vector pGWB35 (empty vector as control).

1. The test plasmid was introduced into Agrobacterium strain GV3101 to obtain recombinant Agrobacterium.

2. the recombinant Agrobacterium obtained in step 1 were resuspended in a solution, to obtain a bacterial suspension with an OD600 nm=1. The solution contained 10 mM MES (2-(N-morphine) ethanesulfonic acid), 10 mM MgCl2 and 200 μmol/L acetosyringone

3. Tobacco plants grown to the 4-6 leaf stage were used to inject the bacterial suspension obtained in step 2 onto the back of tobacco leaves (2-3 leaves of each tobacco plant were inoculated by inoculation, the injection volume per leaf is 200-300 μl).

4. The tobacco plants after completion of step 3, were kept in the dark for 24 hours, then subjected to light culture for 36 hours, at about 22)C

5. After step 4, the leaves of the tobacco plants were cut and cultured on MS medium flat and 20 μL of a substrate solution (Beetle Luciferin (Potassium Salt, Promega, cat # E1601) diluted to 10 volumes with sterile ddH2O water.) was applied to the entire inoculation area and left in the dark for 2-3 min. Afterwards the plant imaging system was used to obtain photographs and allow fluorescence value calculation.

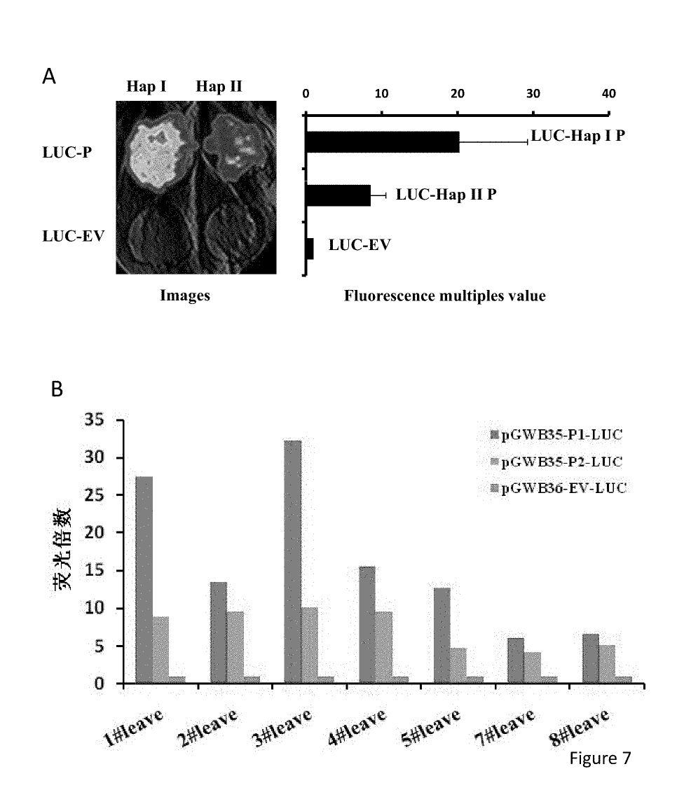

The results are shown in FIG. 7. In FIG. 7, P1 represents the recombinant plasmid-P1, P2 represents the recombinant plasmid-P2, and EV represents the vector pGWB35. In Panel B, the corresponding fluorescence value of the vector pGWB35 is 1, the vertical axis is the fluorescence multiple, and the numbers 1 # to 8 # respectively represent different leaves. The fluorescence generated by P1 promoter was significantly higher than that by P2 promoter. In some leaves, the activity of P1 promoter was more than 3 times higher than that of P2 promoter. The results showed that both P1 and P2 were active promoters, but the P1 promoter had a significantly higher promoter activity than the P2 promoter. The images in FIG. 7 panel A (HAPI corresponding to P1 and Hap II corresponding to P2) show a similar result.

### Example 5. Identification of SNPs in the Promoter Region of TaTPP-7A and Correlation to Grain Traits in Various Wheat Varieties

There are 5 SNP differences between P1 promoter (SEQ ID No: 14) and P2 promoter. (SEQ ID No: 15). Using the P1 promoter as a standard, the P2 promoter differs in the following nucleotide positions:
1) Insertion of a nucleotide “C” between the 409th and 410th nucleotides;
2) SNP at the 493th nucleotide of SEQ ID No: 14: the polymorphic form is T/C (T in SEQ ID No: 14; C in SEQ ID No. 15)
3) SNP at the nucleotide of 1208 of SEQ ID No: 14, the polymorphic form is A/G (A in SEQ ID No: 14; Gin SEQ ID No. 15);
4) SNP at the 1708th nucleotide, the polymorphic form is T/G; (T in SEQ ID No: 14; G in SEQ ID No. 15)
5) SNP at the 1980th nucleotide, the polymorphic form is G/A (G in SEQ ID No: 14; A in SEQ ID No. 15)

5. The wheat lines for testing were planted in the yard of the Institute of Crop Science, Chinese Academy of Agricultural Sciences in October 2012, subjected to conventional irrigation and fertilization management, grains were harvested in July 2013 and their thousand-kernel weight was measured.

The thousand-kernel weight of each wheat material for testing is shown in Table 2.

TABLE 2

| | | | Promoter | Genotype | Genotype |
|---|---|---|---|---|---|
| No. | Name | TGW | type | SNP488 | SNP2144 |
| C1 | Zhongyou 9507 | 51.7 g | P1 | AA | AA |
| C2 | Zhengmai 9023 | 44.1 g | P1 | AA | AA |
| C3 | Pan 86001-3 | 52.8 g | P1 | AA | AA |
| C4 | Jinmai No. 8 | 41.3 g | P1 | AA | AA |
| C5 | Laizhou 953 | 42.05 g | P1 | AA | AA |
| C6 | Xiaobaimang | 44.42 g | P1 | AA | AA |
| C7 | Sankecun | 53.66 g | P1 | AA | AA |
| C8 | Zijiehong | 44.35 g | P1 | AA | AA |
| C9 | Hongmangzi | 37.54 g | P1 | AA | AA |
| C10 | Yuqiumai | 44.29 g | P1 | AA | AA |
| C11 | Lumai No. 1 | 45.658 g | P1 | AA | AA |
| C12 | Beijing 15 | 28.55 g | P2 | CC | TT |
| C13 | Shijiazhuang | 33.28 g | P2 | CC | TT |
| | 54 | | | | |
| C14 | Xuzhou 22 | 51.3 g | P1 | AA | AA |
| C15 | Wenmai No. 8 | 51.7 g | P1 | AA | AA |
| C16 | Lankao 906 | 51.7 g | P1 | AA | AA |
| C17 | Aifeng No. 3 | 34.464 g | P2 | CC | TT |
| C18 | Lumai No. 9 | 26.45 g | P2 | CC | TT |
| C19 | Mingxian 169 | 33.2 g | P2 | CC | TT |
| C20 | Anhui No. 3 | 18.29 g | P2 | CC | TT |
| C21 | Qiangchangmai | 30.4 g | P2 | CC | TT |
| C22 | Baidongmai | 15.75 g | P2 | CC | TT |
| C23 | Lanhuamai | 28.6 g | P2 | CC | TT |
| C24 | Baimangmai | 29.85 g | P2 | CC | TT |
| C25 | Baihuamai | 24.45 g | P2 | CC | TT |
| C26 | Chinese Spring | 27.35 g | P2 | CC | TT |
| C27 | Lvhan 328 | 33.7 g | P2 | CC | TT |
| C28 | Nongda 139 | 32.05 g | P1 | AA | AA |
| C29 | Jingyang 60 | 27.3 g | P2 | CC | TT |
| C30 | Yannong 15 | 34.05 g | P2 | CC | TT |
| C31 | Baimaizi | 24.45 g | P2 | CC | TT |
| C32 | Mahuaban | 20.9 g | P2 | CC | TT |
| C33 | Hongjinmai | 23.4 g | P2 | CC | TT |
| C34 | Sanyuehuang | 28.85 g | P2 | CC | TT |

Among the 34 tested wheat cultivars, 15 genotypes were homozygous for the P1 promoter, and 19 were homozygous for the P2 promoter. The average thousand-kernel weight of grains in wheat comprising the P1 promoter was 45.91 g, and the average thousand-kernel weight of grains in wheat comprising the P2 promoter was 27.54 g.

Using a thousand-kernel weight of 35 g as threshold, the wheat having a thousand-kernel weight of above 35 g was called wheat of high thousand-kernel weight, and the wheat having a thousand-kernel weight lower than 35 g was called wheat of low thousand-kernel weight. If the genotype of the wheat to be tested is homozygous for the P1 promoter, the wheat line is classified as candidate for wheat of high thousand-kernel weight; If the genotype of the wheat to be tested is homozygous for the P1 promoter, the wheat line to be tested is classified as candidate for wheat of low thousand-kernel weight. The accuracy of this method for identification of wheat of high thousand-kernel weight from the 34 tested wheat samples was 93% (14/15), and the accuracy of this method for identification of wheat of low thousand-kernel weight from the 34 tested wheat samples was 100% (19/19).

In 2002, 2005 and 2006, the wheat materials for testing were planted in Luoyang, Henan, and subjected to conventional water and fertilizer management. The grains were harvested and measured in terms of thousand-kernel weight (TKW), kernel length (KL) and kernel width (KW).

The results for tested wheat materials of P1 genotype were as shown in Table 3 (including the results for each tested wheat, and the average value for all the tested wheat having said genotype). The results for tested wheat materials of P1/P2 genotype were as shown in Table 4 (including the results for each tested wheat, and the average value for all the tested wheat having said genotype). The results for tested wheat materials of P2 genotype were as shown in Table 5 (including the results for each tested wheat, and the average value for all the tested wheat having said genotype). From the general trend, the wheat of the P1 genotype had a heavier thousand-kernel weight than the wheat of P2 genotype, and the wheat of P1 genotype had a longer kernel length than the wheat of P2 genotype.

A thousand-kernel weight ≥35 g was defined as high thousand-kernel weight; a thousand-kernel weight <35 g was defined as low thousand-kernel weight. A kernel length ≥0.65 mm was defined as long kernel length; kernel length <0.65 mm was defined as short kernel length. The wheat of P1 genotype was identified as wheat of high thousand-kernel weight, long kernel length, with the accuracy result being shown in Table 3. The wheat of P2 genotype was identified as wheat of low thousand-kernel weight, short kernel length, with the accuracy result being shown in Table 5.

TABLE 3

| | | Genotype | | | | | | | | | |
|---|---|---|---|---|---|---|---|---|---|---|---|
| | | SNP488 | 2006 | | | 2005 | | | 2002 | | |
| | | Promoter | TKW | KL | KD | TKW | KL | KD | TKW | KL | KD |
| | Bank No. | SNP2144 | (g) | (mm) | (mm) | (g) | (mm) | (mm) | (g) | (mm) | (mm) |
| Dahongmai | ZM010600 | AA | 50.764 | 0.79 | 0.33 | 31.71 | 0.79 | 0.32 | 53.4 | 0.79 | 0.335 |
| | | P1 | | | | | | | | | |
| | | AA | | | | | | | | | |
| Laomai | ZM003512 | AA | 37.338 | 0.746667 | 0.3 | | | | 39.96 | 0.66 | 0.27 |
| | | P1 | | | | | | | | | |
| | | AA | | | | | | | | | |
| Xiaobaimang | ZM000556 | AA | 43.646 | 0.67 | 0.34 | 45.19 | 0.673333 | 0.34 | 34.88 | 0.7 | 0.3 |
| | | P1 | | | | | | | | | |
| | | AA | | | | | | | | | |
| Zhongyou | Unknown | AA | 53.428 | 0.78 | 0.34 | 48.845 | 0.776667 | 0.33 | | 0.79 | 0.36 |
| 9507 | | P1 | | | | | | | | | |
| | | AA | | | | | | | | | |
| Jinmai No.8 | ZM009368 | AA | 44.738 | 0.673333 | 0.33 | 38.69 | 0.656667 | 0.32 | 42.62 | 0.63 | 0.31 |
| (Jinzhong 849) | | P1 | | | | | | | | | |
| | | AA | | | | | | | | | |
| Fengkang No. 2 | ZM013100 | AA | 42.376 | 0.67 | 0.31 | 41.5 | 0.656667 | 0.35 | 43.7 | 0.63 | 0.33 |
| (5248) | | P1 | | | | | | | | | |
| | | AA | | | | | | | | | |
| Changzhi 6406 | ZM014022 | AA | 47.002 | 0.703333 | 0.33 | 46.94 | 0.713333 | 0.35 | 52.7 | 0.58 | 0.335 |
| | | P1 | | | | | | | | | |
| | | AA | | | | | | | | | |
| Beijing No.8 | ZM008963 | AA | 38.354 | 0.64 | 0.33 | 33.27 | 0.616667 | 0.32 | 37.06 | 0.63 | 0.32 |
| | | P1 | | | | | | | | | |
| | | AA | | | | | | | | | |
| Yan'an 11 | ZM009627 | AA | 42.754 | 0.713333 | 0.32 | 40.65 | 0.726667 | 0.31 | 41.3 | 0.77 | 0.305 |
| | | P1 | | | | | | | | | |
| | | AA | | | | | | | | | |
| Nongda 183 | ZM009027 | AA | 34.962 | 0.636667 | 0.29 | 31.5 | 0.63 | 0.29 | 33.96 | 0.605 | 0.29 |
| | | P1 | | | | | | | | | |
| | | AA | | | | | | | | | |
| Nongda 311 | ZM009028 | AA | 35.128 | 0.626667 | 0.3 | 35.655 | 0.63 | 0.3 | 41.62 | 0.635 | 0.31 |
| | | P1 | | | | | | | | | |
| | | AA | | | | | | | | | |
| Nongda 139 | ZM009018 | AA | 36.646 | 0.71 | 0.29 | 31.495 | 0.716667 | 0.28 | 35.48 | 0.66 | 0.295 |
| | | P1 | | | | | | | | | |
| | | AA | | | | | | | | | |
| Dongfanghong | ZM009038 | AA | 35.896 | 0.653333 | 0.32 | 42.92 | 0.686667 | 0.33 | 45.42 | 0.665 | 0.33 |
| No. 3 | | P1 | | | | | | | | | |
| | | AA | | | | | | | | | |
| Dahuangpi | ZM006499 | AA | 33.79 | 0.6 | 0.32 | 30.69 | 0.596667 | 0.3 | 36.9 | 0.64 | 0.315 |
| | | P1 | | | | | | | | | |
| | | AA | | | | | | | | | |
| Sankecun | ZM011213 | AA | 53.656 | 0.79 | 0.32 | 53.935 | 0.816667 | 0.33 | 57.56 | 0.815 | 0.33 |
| | | P1 | | | | | | | | | |
| | | AA | | | | | | | | | |
| Paozimai | ZM007298 | AA | 37.62 | 0.706667 | 0.32 | 35.17 | 0.72 | 0.29 | 39.34 | 0.705 | 0.335 |
| | | P1 | | | | | | | | | |
| | | AA | | | | | | | | | |
| Huadong No.6 | ZM010184 | AA | 34.786 | 0.61 | 0.33 | 35.41 | 0.593333 | 0.32 | 36.82 | 0.62 | 0.35 |
| | | P1 | | | | | | | | | |
| | | AA | | | | | | | | | |
| Sumai No.3 | ZM010242 | AA | 37.698 | 0.643333 | 0.34 | 36.95 | 0.646667 | 0.32 | 39.34 | 0.63 | 0.345 |
| | | P1 | | | | | | | | | |
| | | AA | | | | | | | | | |
| Yangmai 158 | H01094 | AA | 45.07 | 0.696667 | 0.34 | 45.41 | 0.713333 | 0.36 | 49.658 | 0.69 | 0.345 |
| | | P1 | | | | | | | | | |
| | | AA | | | | | | | | | |
| Enmai No.4 | ZM016244 | AA | 45.378 | 0.71 | 0.33 | 42.955 | 0.696667 | 0.32 | 49.2 | 0.63 | 0.365 |
| | | P1 | | | | | | | | | |
| | | AA | | | | | | | | | |
| Emai No.6 | ZM010314 | AA | 40.32 | 0.67 | 0.35 | 42.505 | 0.67 | 0.33 | 42.48 | 0.675 | 0.35 |
| | | P1 | | | | | | | | | |
| | | AA | | | | | | | | | |
| Guangtou | ZM004338 | AA | 30.08 | 0.626667 | 0.29 | 36.61 | 0.65 | 0.28 | 31.5 | 0.615 | 0.32 |
| | | P1 | | | | | | | | | |
| | | AA | | | | | | | | | |
| Kefeng No.3 | ZM014679 | AA | 37.244 | 0.613333 | 0.32 | 33.6 | 0.603333 | 0.31 | 36.2 | 0.595 | 0.32 |
| | | P1 | | | | | | | | | |
| | | AA | | | | | | | | | |
| Xinshuguang | ZM009662 | AA | 37.676 | 0.663333 | 0.34 | 41.02 | 0.68 | 0.34 | 42.56 | 0.63 | 0.305 |
| No. 6 | | P1 | | | | | | | | | |
| | | AA | | | | | | | | | |
| Akagomughi | MY000019 | AA | 43.668 | 0.72 | 0.35 | 39.425 | 0.72 | 0.34 | 48.58 | 0.71 | 0.345 |
| (Chixiaomai) | | P1 | | | | | | | | | |
| | | AA | | | | | | | | | |
| Funo (Afu) | MY001072 | AA | 36.414 | 0.646667 | 0.33 | 34.16 | 0.643333 | 0.32 | 37.1 | 0.62 | 0.31 |
| | | P1 | | | | | | | | | |
| | | AA | | | | | | | | | |
| KaBka3 | MY003290 | AA | 33.79 | 0.626667 | 0.34 | 28.915 | 0.606667 | 0.31 | 35.78 | 0.61 | 0.335 |
| (Gaojiasuo) | | P1 | | | | | | | | | |
| | | AA | | | | | | | | | |
| St 2422/464 | MY002776 | AA | 41.088 | 0.69 | 0.32 | 40.31 | 0.7 | 0.33 | 38.38 | 0.69 | 0.31 |
| (Zhengyin No. 4) | | P1 | | | | | | | | | |
| | | AA | | | | | | | | | |
| Mentana (Nanda | MY001904 | AA | 38.772 | 0.706667 | 0.32 | 44.145 | 0.723333 | 0.32 | 34.82 | 0.68 | 0.34 |
| 2419) | | P1 | | | | | | | | | |
| | | AA | | | | | | | | | |
| Orofen (Ourou) | MY002255 | AA | 34.2 | 0.67 | 0.29 | 32.755 | 0.676667 | 0.3 | 37.5 | 0.67 | 0.3 |
| | | P1 | | | | | | | | | |
| | | AA | | | | | | | | | |
| Nonglin No. 10 | MY000054 | AA | 36.288 | 0.676667 | 0.34 | 38.645 | 0.676667 | 0.32 | 33.7 | 0.655 | 0.31 |
| | | P1 | | | | | | | | | |
| | | AA | | | | | | | | | |
| Atlas 66 (Atelasi | MY000295 | AA | 41.156 | 0.713333 | 0.33 | 36.705 | 0.713333 | 0.32 | 39.48 | 0.685 | 0.305 |
| 66) | | P1 | | | | | | | | | |
| | | AA | | | | | | | | | |
| Taishan No. 1 | ZM009405 | AA | 42.902 | 0.686667 | 0.34 | 41.53 | 0.68 | 0.32 | 43.68 | 0.735 | 0.355 |
| | | P1 | | | | | | | | | |
| | | AA | | | | | | | | | |
| Ji'nan No. 2 | ZM009391 | AA | 41.756 | 0.646667 | 0.32 | 40.1 | 0.643333 | 0.33 | 41.94 | 0.685 | 0.34 |
| | | P1 | | | | | | | | | |
| | | AA | | | | | | | | | |
| Youbao | ZM009411 | AA | 38.578 | 0.663333 | 0.31 | 35.84 | 0.696667 | 0.3 | 36.16 | 0.635 | 0.31 |
| | | P1 | | | | | | | | | |
| | | AA | | | | | | | | | |
| Xiannong 39 | ZM017208 | AA | 42.722 | 0.726667 | 0.32 | 38.39 | 0.736667 | 0.31 | | 0.715 | 0.33 |
| | | P1 | | | | | | | | | |
| | | AA | | | | | | | | | |
| Ji'nan 17 | Unknown | AA | 42.72 | 0.683333 | 0.35 | 41.225 | 0.71 | 0.33 | | 0.71 | 0.325 |
| | | P1 | | | | | | | | | |
| | | AA | | | | | | | | | |
| Xiaoyan No. 6 | ZM017079 | AA | 41.712 | 0.68 | 0.32 | 39.165 | 0.66 | 0.32 | 40.76 | 0.68 | 0.355 |
| | | P1 | | | | | | | | | |
| | | AA | | | | | | | | | |
| Shannong 7859 | ZM017231 | AA | 50.572 | 0.783333 | 0.35 | 46.765 | 0.786667 | 0.33 | 55.2 | 0.815 | 0.385 |
| | | P1 | | | | | | | | | |
| | | AA | | | | | | | | | |
| Lumai No.1 | ZM015830 | AA | 45.658 | 0.6%667 | 0.37 | 43.67 | 0.69 | 0.36 | 47.64 | 0.73 | 0.345 |
| (Aimengniu) | | P1 | | | | | | | | | |
| | | AA | | | | | | | | | |
| Laizhou 953 | ZM022727 | AA | 51.328 | 0.706667 | 0.36 | 49.91 | 0.68 | 0.35 | 52.2 | 0.66 | 0.355 |
| | | P1 | | | | | | | | | |
| | | AA | | | | | | | | | |
| Zijiehong | ZM002272 | AA | 43.944 | 0.666667 | 0.32 | 44.76 | 0.673333 | 0.34 | | 0.665 | 0.31 |
| | | P1 | | | | | | | | | |
| | | AA | | | | | | | | | |
| Zangdong No. 4 | ZM010580 | AA | 42.012 | 0.71 | 0.34 | 34.155 | 0.693333 | 0.32 | 40.76 | 0.695 | 0.33 |
| | | P1 | | | | | | | | | |
| | | AA | | | | | | | | | |
| Rikaze No. 8 | ZM010589 | AA | 44.82 | 0.646667 | 0.36 | 42.685 | 0.63 | 0.35 | 42.26 | 0.635 | 0.36 |
| | | P1 | | | | | | | | | |
| | | AA | | | | | | | | | |
| Hongmaimang | ZM020720 | AA | 35.04 | 0.67 | 0.31 | 34.66 | | | | 0.635 | 0.285 |
| | | P1 | | | | | | | | | |
| | | AA | | | | | | | | | |
| Dabaimai | ZM005102 | AA | 39.926 | 0.683333 | 0.33 | 35.84 | 0.65 | 0.31 | 35.86 | 0.63 | 0.29 |
| | | P1 | | | | | | | | | |
| | | AA | | | | | | | | | |
| Baiqitou | ZM012810 | AA | 44.608 | 0.673333 | 0.36 | 39.37 | 0.643333 | 0.34 | 42.12 | 0.695 | 0.345 |
| | | P1 | | | | | | | | | |
| | | AA | | | | | | | | | |
| Ganmai No. 8 | ZM009803 | AA | 48.07 | 0.7 | 0.35 | 44.485 | 0.72 | 0.32 | 50.88 | 0.705 | 0.345 |
| | | P1 | | | | | | | | | |
| | | AA | | | | | | | | | |
| Gaoyuan 506 | ZM010116 | AA | 42.266 | 0.673333 | 0.35 | 30.825 | 0.666667 | 0.33 | 41.68 | 0.675 | 0.335 |
| | | P1 | | | | | | | | | |
| | | AA | | | | | | | | | |
| Qingchun 28 | ZM017383 | AA | 49.622 | 0.72 | 0.35 | 45.41 | 0.723333 | 0.36 | 46.98 | 0.72 | 0.345 |
| | | P1 | | | | | | | | | |
| | | AA | | | | | | | | | |
| Ningchun No. 4 | ZM017424 | AA | 46.684 | 0.656667 | 0.36 | 42.635 | 0.656667 | 0.35 | 48.88 | 0.655 | 0.34 |
| (Yongliang No. 4) | | P1 | | | | | | | | | |
| | | AA | | | | | | | | | |
| Huzhuhong | ZM017354 | AA | 36.462 | 0.64 | 0.32 | 32.215 | 0.62 | 0.3 | 33.5 | 0.635 | 0.325 |
| Jinmai No. 4 | ZM009972 | AA | 51.466 | 0.723333 | 0.34 | 43 | 0.73 | 0.33 | 52.58 | 0.76 | 0.34 |
| | | P1 | | | | | | | | | |
| | | AA | | | | | | | | | |
| Dingxi 24 | ZM009893 | AA | 34.328 | 0.673333 | 0.31 | 31.225 | 0.656667 | 0.29 | 43.24 | 0.71 | 0.31 |
| | | P1 | | | | | | | | | |
| | | AA | | | | | | | | | |
| Shuwan No.8 | ZM010490 | AA | 48.452 | 0.72 | 0.34 | 46.67 | 0.73 | 0.34 | 44.52 | 0.685 | 0.355 |
| | | P1 | | | | | | | | | |
| | | AA | | | | | | | | | |
| Bimai 26 | ZM023312 | AA | 41.96 | 0.723333 | 0.35 | 38.72 | 0.716667 | 0.32 | | 0.735 | 0.295 |
| | | P1 | | | | | | | | | |
| | | AA | | | | | | | | | |
| Guinong No.10 | ZM023371 | AA | 47.936 | 0.696667 | 0.34 | 39.8 | 0.683333 | 0.32 | | 0.715 | 0.305 |
| | | P1 | | | | | | | | | |
| | | AA | | | | | | | | | |
| Yunmai 34 | ZM016965 | AA | 44.288 | 0.7 | 0.31 | | 0.686667 | 0.32 | 41.9 | 0.715 | 0.305 |
| | | P1 | | | | | | | | | |
| | | AA | | | | | | | | | |
| Xingyi No. 4 | ZM023315 | AA | 49.84 | 0.783333 | 0.34 | 48.32 | 0.75 | 0.32 | | 0.785 | 0.31 |
| | | P1 | | | | | | | | | |
| | | AA | | | | | | | | | |
| Fengmai 11 | ZM010564 | AA | 44.156 | 0.713333 | 0.32 | 45.775 | 0.69 | 0.33 | 45.74 | 0.71 | 0.33 |
| | | P1 | | | | | | | | | |
| | | AA | | | | | | | | | |
| Hongmangzi | ZM020144 | AA | 39.69 | 0.663333 | 0.33 | 35.38 | 0.66 | 0.3 | 48.02 | 0.715 | 0.355 |
| | | P1 | | | | | | | | | |
| | | AA | | | | | | | | | |
| Yuqiumai | ZM008636 | AA | 37.16 | 0.603333 | 0.34 | 28.9 | 0.593333 | 0.3 | | 0.605 | 0.31 |
| | | P1 | | | | | | | | | |
| | | AA | | | | | | | | | |
| Hongdongmai | ZM005188 | AA | 26.644 | 0.636667 | 0.29 | 25.595 | 0.656667 | 0.28 | | 0.72 | 0.235 |
| | | P1 | | | | | | | | | |
| | | AA | | | | | | | | | |
| Wumangchunmai | ZM005336 | AA | 33.368 | 0.663333 | 0.31 | 34.06 | 0.636667 | 0.3 | 30.76 | 0.67 | 0.26 |
| | | P1 | | | | | | | | | |
| | | AA | | | | | | | | | |
| Xindong No. 2 | ZM010146 | AA | 30.956 | 0.6 | 0.33 | 37.045 | 0.64 | 0.33 | 39.2 | 0.6 | 0.335 |
| | | P1 | | | | | | | | | |
| | | AA | | | | | | | | | |
| Zhengmai 9023 | Unknown | AA | 51.762 | 0.723333 | 0.36 | 49.51 | 0.73 | 0.31 | | | |
| | | P1 | | | | | | | | | |
| | | AA | | | | | | | | | |
| Yanzhan No. 1 | Unknown | AA | 49.358 | 0.673333 | 0.36 | | | | | | |
| | | P1 | | | | | | | | | |
| | | AA | | | | | | | | | |

| | | | | | | | | | |
|---|---|---|---|---|---|---|---|---|---|
| Average value | 41.5596 | 0.6836 | 0.33 | 39.1455 | 0.6819 | 0.3213 | 42.0992 | 0.6792 | 0.3243 |
| Accuracy | 50/67 | 51/67 | | 47/64 | 48/64 | | 38/45 | 44/65 | |

TABLE 4

| | | Genotype | | | | | | | | | |
|---|---|---|---|---|---|---|---|---|---|---|---|
| | | SNP488 | 2006 | | | 2005 | | | 2002 | | |
| | | Promoter | TKW | KL | KW | TKW | KL | KW | TKW | KL | KW |
| | Bank No. | SNP2144 | (g) | (mm) | (mm) | (g) | (mm) | (mm) | (g) | (mm) | (mm) |
| Jinghong No.5 | ZM008934 | AC | 44.94 | 0.76 | 0.34 | 46.36 | | | 47.8 | | |
| | | PJ/P2 | | | | | | | | | |
| | | AT | | | | | | | | | |
| Youmangbaifu | ZM004418 | AC | 22.646 | 0.59 | 0.26 | 22.625 | 0.583333 | 0.28 | | 0.55 | 0.255 |
| | | PJ/P2 | | | | | | | | | |
| | | AT | | | | | | | | | |
| Wangshuibai | ZM005740 | AC | 39.748 | 0.693333 | 0.31 | 34.235 | 0.676667 | 0.31 | 42.62 | 0.645 | 0.335 |
| | | PJ/P2 | | | | | | | | | |
| | | AT | | | | | | | | | |
| Yangmai | ZM004358 | AC | 29.346 | 0.616667 | 0.31 | 30.325 | 0.623333 | 0.3 | 32.66 | 0.585 | 0.3 |
| | | PJ/P2 | | | | | | | | | |
| | | AT | | | | | | | | | |
| Dunhuachunmai | ZM010769 | AC | 32.748 | 0.61 | 0.32 | 31.065 | 0.623333 | 0.31 | 35.98 | 0.645 | 0.32 |
| | | PJ/P2 | | | | | | | | | |
| | | AT | | | | | | | | | |
| Daqingmang | ZM010715 | AC | 27.8 | 0.66 | 0.29 | | 0.68 | 0.28 | 30.1 | 0.635 | 0.295 |
| | | PJ/P2 | | | | | | | | | |
| | | AT | | | | | | | | | |
| Xinkehan No. 9 | ZM022178 | AC | 46.102 | 0.736667 | 0.33 | 42.67 | 0.736667 | 0.32 | 48.6 | 0.695 | 0.315 |
| | | PJ/P2 | | | | | | | | | |
| | | AT | | | | | | | | | |
| Xinshuguang | ZM009657 | AC | 39.366 | 0.716667 | 0.34 | 39.235 | 0.7 | 0.33 | 49.18 | 0.685 | 0.305 |
| No. 1 | | PJ/P2 | | | | | | | | | |
| | | AT | | | | | | | | | |
| Dongnong 101 | ZM009732 | AC | 27.898 | 0.566667 | 0.31 | 29.02 | 0.573333 | 0.3 | 30.14 | 0.56 | 0.31 |
| | | PJ/P2 | | | | | | | | | |
| | | AT | | | | | | | | | |
| Jichun 1016 | ZM021929 | AC | 42.944 | 0.696667 | 0.35 | 42.715 | 0.696667 | 0.34 | 43.4 | 0.655 | 0.305 |
| | | PJ/P2 | | | | | | | | | |
| | | AT | | | | | | | | | |
| Triumph | MY002966 | AC | 38.288 | 0.67 | 0.3 | 39.59 | 0.7 | 0.3 | 41.86 | 0.71 | 0.315 |
| (Shenglimai) | | PJ/P2 | | | | | | | | | |
| | | AT | | | | | | | | | |
| Loynn 10 | MY001759 | AC | 45.8 | 0.67 | 0.32 | 37.715 | 0.676667 | 0.32 | 45.26 | 0.705 | 0.345 |
| (Luofulin | | PJ/P2 | | | | | | | | | |
| No. 10) | | AT | | | | | | | | | |
| Taizhong 23 | ZM013082 | AC | 39.574 | 0.67 | 0.34 | 33.715 | 0.63 | 0.29 | 37.32 | | |
| | | PJ/P2 | | | | | | | | | |
| | | AT | | | | | | | | | |
| Dixiuzao | ZM010368 | AC | 50.39 | 0.733333 | 0.36 | 47.725 | 0.68 | 0.33 | 48.3 | 0.68 | 0.345 |
| | | PJ/P2 | | | | | | | | | |
| | | AT | | | | | | | | | |
| Bima No. 1 | ZM009591 | AC | 39.07 | 0.626667 | 0.34 | 40.12 | 0.643333 | 0.34 | 40.54 | 0.625 | 0.345 |
| | | PJ/P2 | | | | | | | | | |
| | | AT | | | | | | | | | |
| Bainong 3217 | ZM017936 | AC | 39.874 | 0.693333 | 0.33 | 37.55 | 0.713333 | 0.33 | 45.34 | 0.68 | 0.305 |
| | | PJ/P2 | | | | | | | | | |
| | | AT | | | | | | | | | |
| Shijiazhuang | ZM009099 | AC | 35.388 | 0.656667 | 0.31 | 35.655 | 0.663333 | 0.33 | 38.76 | 0.645 | 0.31 |
| 407 | | PJ/P2 | | | | | | | | | |
| | | AT | | | | | | | | | |
| Wenmai No. 6 | ZM025398 | AC | 48.24 | 0.656667 | 0.37 | 47.25 | 0.64 | 0.36 | 47.74 | 0.605 | 0.335 |
| (Yumai 49) | | PJ/P2 | | | | | | | | | |
| | | AT | | | | | | | | | |
| Zhengzhou | ZM015988 | AC | 38.686 | 0.743333 | 0.31 | 37.565 | 0.726667 | 0.32 | 35.72 | 0.725 | 0.315 |
| 741 | | PJ/P2 | | | | | | | | | |
| | | AT | | | | | | | | | |
| Baibiansui | ZM001782 | AC | 34.802 | 0.583333 | 0.33 | 34.08 | 0.59 | 0.32 | 38.96 | 0.585 | 0.305 |
| | | PJ/P2 | | | | | | | | | |
| | | AT | | | | | | | | | |
| Geerhongmai | ZM019809 | AC | 34.932 | 0.723333 | 0.3 | 33.03 | 0.753333 | 0.26 | 37.42 | 0.725 | 0.285 |
| | | PJ/P2 | | | | | | | | | |
| | | AT | | | | | | | | | |
| Jiangmai | ZM011774 | AC | 35.138 | 0.643333 | 0.31 | 28.25 | 0.65 | 0.28 | | 0.61 | 0.3 |
| | | PJ/P2 | | | | | | | | | |
| | | AT | | | | | | | | | |
| Yangmai | ZM011644 | AC | 36.512 | 0.686667 | 0.33 | 38.455 | 0.663333 | 0.32 | 43.68 | 0.68 | 0.33 |
| | | PJ/P2 | | | | | | | | | |
| | | AT | | | | | | | | | |
| Tuokexun | ZM010136 | AC | 36.386 | 0.643333 | 0.33 | 37.19 | 0.666667 | 0.3 | 42.28 | 0.645 | 0.345 |
| No.1 | | PJ/P2 | | | | | | | | | |
| | | AT | | | | | | | | | |

TABLE 5

| | | Genotype | | | | | | | | | |
|---|---|---|---|---|---|---|---|---|---|---|---|
| | | SNP488 | 206 | | | 2005 | | | 2002 | | |
| | | Promoter | TKW | KL | KW | TKW | KL | KW | TKW | KL | KW |
| | Bank No. | SNP2144 | (g) | (mm) | (mm) | (g) | (mm) | (mm) | (g) | (mm) | (mm) |
| Neimai 11 | ZM017834 | CC | 34.934 | 0.613333 | 0.33 | 28.83 | 0.59 | 0.31 | 47.22 | 0.69 | 0.335 |
| | | P2 | | | | | | | | | |
| | | TT | | | | | | | | | |
| Jinchun No. 3 | ZM014440 | CC | 44.373 | 0.67 | 0.35 | 35.585 | 0.63 | 0.32 | 44.16 | 0.61 | 0.33 |
| (Xichun'ai No. 2) | | P2 | | | | | | | | | |
| | | TT | | | | | | | | | |
| Lianglaiyou baipi | ZM009771 | CC | 36.097 | 0.653333 | 0.32 | | | | 34.92 | 0.645 | 0.305 |
| wheat | | P2 | | | | | | | | | |
| | | TT | | | | | | | | | |
| Bihongsui | ZM009772 | CC | 30.56 | 0.63 | 0.3 | | 0.633333 | 0.27 | 31.38 | 0.67 | 0.295 |
| | | P2 | | | | | | | | | |
| | | TT | | | | | | | | | |
| Xiaobaimai | ZM004615 | CC | 30.906 | 0.68 | 0.32 | 38.565 | 0.66 | 0.3 | 35.52 | 0.675 | 0.31 |
| | | P2 | | | | | | | | | |
| | | TT | | | | | | | | | |
| Hongpi wheat | ZM004594 | CC | 30.576 | 0.68 | 0.3 | 25.21 | 0.68 | 0.28 | 28.62 | 0.61 | 0.26 |
| | | P2 | | | | | | | | | |
| | | TT | | | | | | | | | |
| Dabaipi | ZM017481 | CC | 33.512 | 0.686667 | 0.29 | 34.555 | 0.68 | 0.3 | 35.36 | 0.66 | 0.295 |
| | | P2 | | | | | | | | | |
| | | TT | | | | | | | | | |
| Xiaohongpi | ZM004634 | CC | 32.452 | 0.67333 | 0.31 | 41.375 | 0.71 | 0.29 | 27.62 | 0.67 | 0.27 |
| | | P2 | | | | | | | | | |
| | | TT | | | | | | | | | |
| Dingxingzhai | ZM010639 | CC | 27.272 | 0.6133333 | 0.29 | 38.575 | 0.61 | 0.27 | 27.7 | 0.61 | 0.285 |
| | | P2 | | | | | | | | | |
| | | TT | | | | | | | | | |
| Honglidangnianlao | ZM003515 | CC | 31.346 | 0.61 | 0.3 | 30.885 | 0.61 | 0.26 | 32.38 | 0.63 | 0.325 |
| | | P2 | | | | | | | | | |
| | | TT | | | | | | | | | |
| Spring wheat | ZM012632 | CC | 36.908 | 0.67 | 0.33 | 36.625 | 0.666667 | 0.29 | 34.06 | 0.31 | 0.15 |
| | | P2 | | | | | | | | | |
| | | TT | | | | | | | | | |
| Huoliaomai | ZM004550 | CC | 30.88 | 0.65 | 0.31 | 28.445 | 0.643333 | 0.31 | 30.7 | 0.635 | 0.26 |
| | | P2 | | | | | | | | | |
| | | TT | | | | | | | | | |
| Shaanxibaimai | ZM004922 | CC | 28.138 | 0.59 | 0.29 | 25.17 | 0.616667 | 0.28 | | 0.625 | 0.285 |
| | | P2 | | | | | | | | | |
| | | TT | | | | | | | | | |
| Niuzhijia | ZM001259 | CC | 35.25 | 0.696667 | 0.32 | 37.835 | 0.726667 | 0.31 | | 0.74 | 0.305 |
| | | P2 | | | | | | | | | |
| | | TT | | | | | | | | | |
| Mahuaban | ZM004422 | CC | 23.13 | 0.59 | 0.29 | 23.155 | 0.596667 | 0.28 | | 0.59 | 0.26 |
| | | P2 | | | | | | | | | |
| | | TT | | | | | | | | | |
| Jiahongmai | ZM001284 | CC | 32.724 | 0.59 | 0.31 | 31.74 | 0.596667 | 0.31 | | 0.64 | 0.28 |
| | | P2 | | | | | | | | | |
| | | TT | | | | | | | | | |
| Hongjinmai | ZM020735 | CC | 26.206 | 0.563333 | 0.28 | 21.03 | 0.573333 | 0.26 | 35.42 | 0.595 | 0.26 |
| | | P2 | | | | | | | | | |
| | | TT | | | | | | | | | |
| Baiqimai | ZM005012 | CC | 30.674 | 0.586667 | 0.31 | 24.835 | 0.59 | 0.27 | | 0.59 | 0.27 |
| | | P2 | | | | | | | | | |
| | | TT | | | | | | | | | |
| Xiaokouong | ZM004454 | CC | 31.916 | 0.61 | 0.3 | 27.83 | 0.59 | 0.29 | 30.56 | 0.615 | 0.31 |
| | | P2 | | | | | | | | | |
| | | TT | | | | | | | | | |
| Lanhuamai | ZM005017 | CC | 25.818 | 0.546667 | 0.3 | 23.305 | 0.55 | 0.28 | | 0.56 | 0.26 |
| | | P2 | | | | | | | | | |
| | | TT | | | | | | | | | |
| Daimanghongmai | ZM000156 | CC | 29.934 | 0.603333 | 0.29 | 28.925 | 0.62 | 0.3 | 29.58 | 0.64 | 0.31 |
| | | P2 | | | | | | | | | |
| | | TT | | | | | | | | | |
| Zhuoludongmai | ZM000474 | CC | 30.03 | 0.61 | 0.3 | 30.215 | 0.596667 | 0.29 | | 0.58 | 0.3 |
| | | P2 | | | | | | | | | |
| | | TT | | | | | | | | | |
| Hongmai | ZM017549 | CC | 34.498 | 0.626667 | 0.32 | 29.5 | 0.683333 | 0.3 | | 0.635 | 0.31 |
| | | P2 | | | | | | | | | |
| | | TT | | | | | | | | | |
| Honglaomai | ZM004174 | CC | 33.05 | 0.696667 | 0.3 | 33.365 | | | | 0.63 | 0.225 |
| | | P2 | | | | | | | | | |
| | | TT | | | | | | | | | |
| Hongpidongmai | ZM001138 | CC | 29.084 | 0.643333 | 0.31 | 28.355 | 0.613333 | 0.29 | | 0.64 | 0.265 |
| | | P2 | | | | | | | | | |
| | | TT | | | | | | | | | |
| Panshiwumang | ZM004412 | CC | 28.148 | 0.653333 | 0.32 | | | | 27.64 | 0.595 | 0.28 |
| | | P2 | | | | | | | | | |
| | | TT | | | | | | | | | |
| Youmangbaifu | ZM004444 | CC | 23.542 | 0.58 | 0.29 | 21.565 | 0.586667 | 0.27 | | 0.585 | 0.245 |
| | | P2 | | | | | | | | | |
| | | TT | | | | | | | | | |
| Baiqiumai | ZM000540 | CC | 29.296 | 0.64 | 0.28 | 28.61 | 0.673333 | 0.27 | | 0.645 | 0.295 |
| | | P2 | | | | | | | | | |
| | | TT | | | | | | | | | |
| Yuandong 822 | ZM013548 | CC | 38.424 | 0.656667 | 0.31 | 38.145 | 0.63 | 0.32 | 41.2 | 0.645 | 0.315 |
| | | P2 | | | | | | | | | |
| | | TT | | | | | | | | | |
| Lvhan 328 | ZM014050 | CC | 37.95 | 0.626667 | 0.33 | 33.655 | 0.606667 | 0.32 | 38 | 0.695 | 0.345 |
| | | P2 | | | | | | | | | |
| | | TT | | | | | | | | | |
| Mingxian 169 | ZM009379 | CC | 31.652 | 0.61 | 0.32 | 27.885 | 0.656667 | 0.32 | 30.54 | 0.63 | 0.305 |
| | | P2 | | | | | | | | | |
| | | TT | | | | | | | | | |
| Xianmai | ZM003498 | CC | 30.528 | 0.603333 | 0.3 | 28.285 | 0.583333 | 0.28 | | 0.63 | 0.285 |
| | | P2 | | | | | | | | | |
| | | TT | | | | | | | | | |
| Jianxizao | ZM003464 | CC | 27.064 | 0.616667 | 0.29 | 25.665 | 0.58 | 0.25 | | 0.615 | 0.29 |
| | | P2 | | | | | | | | | |
| | | TT | | | | | | | | | |
| Honghuazao | ZM011345 | CC | 27.51 | 0.56 | 0.28 | 20.885 | 0.543333 | 0.27 | 23.9 | 0.57 | 0.28 |
| | | P2 | | | | | | | | | |
| | | TT | | | | | | | | | |
| Jiangdongmen | ZM005871 | CC | 31.22 | 0.613333 | 0.29 | 27.03 | 0.586667 | 0.29 | 29.92 | 0.625 | 0.31 |
| | | P2 | | | | | | | | | |
| | | TT | | | | | | | | | |
| Chongyanghongmai 1 | ZM011446 | CC | 31.096 | 0.636667 | 0.3 | 26.04 | 0.606667 | 0.26 | 32.8 | 0.62 | 0.33 |
| | | P2 | | | | | | | | | |
| | | TT | | | | | | | | | |
| Zaowutian | ZM005992 | CC | 29.49 | 0.576667 | 0.29 | 26.165 | 0.553333 | 0.3 | 26.8 | 0.58 | 0.305 |
| | | P2 | | | | | | | | | |
| | | TT | | | | | | | | | |
| Liuzhutou | ZM005540 | CC | 35.82 | 0.63 | 0.32 | 28.925 | 0.586667 | 0.3 | 34.58 | 0.64 | 0.295 |
| | | P2 | | | | | | | | | |
| | | TT | | | | | | | | | |
| Chanbuzi | ZM006465 | CC | 37.63 | 0.653333 | 0.33 | 32.77 | 0.623333 | 0.31 | 41.46 | | |
| | | P2 | | | | | | | | | |
| | | TT | | | | | | | | | |
| Zhumaoyuaniou | ZM006479 | CC | 27.14 | 0.573333 | 0.31 | 16.465 | 0.536667 | 0.29 | 27.4 | 0.55 | 0.285 |
| | | P2 | | | | | | | | | |
| | | TT | | | | | | | | | |
| Shuilizhan | ZM007343 | CC | 33.524 | 0.633333 | 0.32 | 30.17 | 0.636667 | 0.29 | 34.68 | 0.635 | 0.325 |
| | | P2 | | | | | | | | | |
| | | TT | | | | | | | | | |
| Huangshuibai | ZM010980 | CC | 32.004 | 0.603333 | 0.3 | 28.355 | 0.59 | 0.31 | 32.78 | 0.65 | 0.325 |
| | | P2 | | | | | | | | | |
| | | TT | | | | | | | | | |
| Baipu (Luoqing) | ZM007246 | CC | 37.374 | 0.663333 | 0.31 | 29.245 | 0.626667 | 0.28 | 36.82 | 0.63 | 0.325 |
| | | P2 | | | | | | | | | |
| | | TT | | | | | | | | | |
| Zaoxianomai | ZM007209 | CC | 39.07 | 0.626667 | 0.34 | 29.91 | 0.616667 | 0.3 | 35.12 | 0.57 | 0.315 |
| | | P2 | | | | | | | | | |
| | | TT | | | | | | | | | |
| Lanxi zaoxiaomai | ZM007052 | CC | 33.296 | 0.61 | 0.32 | 28.205 | 0.606667 | 0.29 | 35.74 | 0.585 | 0.32 |
| | | P2 | | | | | | | | | |
| | | TT | | | | | | | | | |
| Wuyuanmai | ZM011087 | CC | 36.156 | 0.7 | 0.3 | | | | 39.28 | 0.66 | 0.305 |
| | | P2 | | | | | | | | | |
| | | TT | | | | | | | | | |
| Chejianzi | ZM006027 | CC | 30.922 | 0.646667 | 0.31 | 28.875 | 0.66 | 0.28 | 31.66 | 0.6 | 0.285 |
| | | P2 | | | | | | | | | |
| | | TT | | | | | | | | | |
| Heshangmai | ZM007486 | CC | 29.408 | 0.60667 | 0.3 | 22.75 | 0.576667 | 0.26 | 32.44 | 0.545 | 0.29 |
| | | P2 | | | | | | | | | |
| | | TT | | | | | | | | | |
| Nuomai | ZM007438 | CC | 32.982 | 0.656667 | 0.32 | 31.66 | 0.63 | 0.3 | 37.96 | 0.66 | 0.31 |
| | | P2 | | | | | | | | | |
| | | TT | | | | | | | | | |
| Mangxiaomai | ZM006015 | CC | 28.694 | 0.616667 | 0.23 | 22.67 | 0.58 | 0.25 | 27.98 | 0.67 | 0.3 |
| | | P2 | | | | | | | | | |
| | | TT | | | | | | | | | |
| Liying No. 5 | ZM015113 | CC | 44.854 | 0.673333 | 0.32 | 39.72 | 0.653333 | 0.32 | 49.36 | 0.66 | 0.36 |
| | | P2 | | | | | | | | | |
| | | TT | | | | | | | | | |
| Anhui No. 3 | ZM010261 | CC | 36.57 | 0.623333 | 0.34 | | | | 42.96 | 0.614 | 0.34 |
| | | P2 | | | | | | | | | |
| | | TT | | | | | | | | | |
| Zhemai No. 1 | H01219 | CC | 31.832 | 0.64 | 0.31 | 29.87 | 0.613333 | 0.31 | 32.6 | 0.625 | 0.315 |
| | | P2 | | | | | | | | | |
| | | TT | | | | | | | | | |
| Baiyoumai | ZM004326 | CC | 27.556 | 0.62 | 0.32 | | 0.646667 | 0.31 | 30.56 | 0.62 | 0.27 |
| | | P2 | | | | | | | | | |
| | | TT | | | | | | | | | |
| Huoqiu | ZM004433 | CC | 33.366 | 0.66 | 0.3 | 31.015 | 0.686667 | 0.29 | 31.3 | 0.655 | 0.3 |
| | | P2 | | | | | | | | | |
| | | TT | | | | | | | | | |
| Kelao No. 4 | ZM014682 | CC | 38.26 | 0.663333 | 0.31 | 34.57 | 0.65 | 0.28 | 39.24 | 0.62 | 0.295 |
| | | P2 | | | | | | | | | |
| | | TT | | | | | | | | | |
| Suwon 86 | MY000140 | CC | 28.388 | 0.66 | 0.28 | 24.495 | 0.643333 | 0.26 | | 0.575 | 0.21 |
| (Shuiyuan 86) | | P2 | | | | | | | | | |
| | | TT | | | | | | | | | |
| Cheyenne × early | MY000663 | CC | 36.684 | 0.673333 | 0.3 | 31.44 | 0.673333 | 0.27 | 37.78 | 0.675 | 0.3 |
| Blackhull | | P2 | | | | | | | | | |
| (Qianjianmai) | | TT | | | | | | | | | |
| Early Premium | MY000898 | CC | 37.094 | 0.686667 | 0.29 | 33.76 | 0.66 | 0.3 | 40.38 | 0.66 | 0.295 |
| (Zaoyangmai) | | P2 | | | | | | | | | |
| | | TT | | | | | | | | | |
| Odessa No. 3 | MY003347 | CC | 32.934 | 0.626667 | 0.3 | 28.445 | 0.616667 | 0.28 | 33.16 | 0.605 | 0.305 |
| | | P2 | | | | | | | | | |
| | | TT | | | | | | | | | |
| Tanori F71 | MY002877 | CC | 36.586 | | | | 0.68 | 0.28 | 34.32 | 0.65 | 0.295 |
| (Taruorui F71) | | P2 | | | | | | | | | |
| | | TT | | | | | | | | | |
| Villa Glori | MY003061 | CC | 35.414 | 0.693333 | 0.32 | 26.5 | 0.703333 | 0.28 | 33.28 | 0.61 | 0.305 |
| (Zhongnong 28) | | P2 | | | | | | | | | |
| | | TT | | | | | | | | | |
| CI12203 (Gansu | ZM009832 | CC | 29.114 | 0.573333 | 0.32 | 25.76 | 0.59 | 0.31 | 31.36 | 0.605 | 0.315 |
| 96) | | P2 | | | | | | | | | |
| | | TT | | | | | | | | | |
| Chaoan wheat | ZM007601 | CC | 36.442 | 0.623333 | 0.3 | 26.605 | 0.653333 | 0.3 | 32.08 | 0.605 | 0.3 |
| | | P2 | | | | | | | | | |
| | | TT | | | | | | | | | |
| Chike | ZM007616 | CC | 37.876 | 0.68 | 0.33 | 31.865 | 0.663333 | 0.29 | 33.64 | 0.625 | 0.285 |
| | | P2 | | | | | | | | | |
| | | TT | | | | | | | | | |
| Songnuimai (No. 4) | ZM007552 | CC | 42.028 | 0.643333 | 0.34 | 28.105 | 0.606667 | 0.28 | 34.62 | 0.575 | 0.345 |
| | | P2 | | | | | | | | | |
| | | TT | | | | | | | | | |
| Shengan | ZM007521 | CC | 40.77 | 0.67 | 0.32 | 33.465 | 0.65 | 0.3 | 41.38 | 0.675 | 0.345 |
| | | P2 | | | | | | | | | |
| | | TT | | | | | | | | | |
| Shanglin wheat | ZM007719 | CC | 35.276 | 0.696667 | 0.3 | 26.525 | 0.7 | 0.25 | 27.42 | 0.545 | 0.275 |
| | | P2 | | | | | | | | | |
| | | TT | | | | | | | | | |
| Kangxiu No. 10 | ZM010352 | CC | 35.046 | 0.626667 | 0.33 | 34.86 | 0.626667 | 0.33 | 38.86 | | |
| | | P2 | | | | | | | | | |
| | | TT | | | | | | | | | |
| Jinmai 2148 | ZM010375 | CC | 47.118 | 0.716667 | 0.35 | 41.305 | 0.703333 | 0.33 | 46.66 | 0.66 | 0.33 |
| | | P2 | | | | | | | | | |
| | | TT | | | | | | | | | |
| Jingyang 60 (Xibei | ZM009648 | CC | 27.696 | 0.54 | 0.32 | 23.455 | 0.556667 | 0.31 | 23.5 | 0.53 | 0.295 |
| 60) | | P2 | | | | | | | | | |
| | | TT | | | | | | | | | |
| Shite 14 | ZM009097 | CC | 31 | 0.636667 | 0.27 | 24.415 | 0.623333 | 0.27 | 33.18 | 0.605 | 0.325 |
| | | P2 | | | | | | | | | |
| | | TT | | | | | | | | | |
| Fuzhuang 30 | ZM017213 | CC | 28.846 | 0.59 | 0.3 | | | | 28.62 | 0.595 | 0.315 |
| | | P2 | | | | | | | | | |
| | | TT | | | | | | | | | |
| Bima No. 4 | ZM009594 | CC | 38.726 | 0.66 | 0.33 | 37.71 | 0.643333 | 0.31 | 29.84 | 0.585 | 0.305 |
| | | P2 | | | | | | | | | |
| | | TT | | | | | | | | | |
| Shijiazhuang 54 | ZM009101 | CC | 36.944 | 0.616667 | 0.32 | 29.625 | 0.606667 | 0.3 | 37.94 | 0.63 | 0.32 |
| | | P2 | | | | | | | | | |
| | | TT | | | | | | | | | |
| Pingyang 27 | ZM014027 | CC | 48.19 | 0.673333 | 0.33 | 45.155 | 0.653333 | 0.34 | 50.18 | 0.705 | 0.356 |
| | | P2 | | | | | | | | | |
| | | TT | | | | | | | | | |
| Fengchan No. 3 | ZM009600 | CC | 38.068 | 0.66 | 0.31 | 40.07 | 0.66 | 0.31 | 37.04 | 0.61 | 0.31 |
| | | P2 | | | | | | | | | |
| | | TT | | | | | | | | | |
| Yannong 15 | ZM015719 | CC | 36.012 | 0.556667 | 0.34 | 35.72 | 0.57 | 0.32 | 34.2 | 0.565 | 0.33 |
| | | P2 | | | | | | | | | |
| | | TT | | | | | | | | | |
| Xinong 6028 | ZM009597 | CC | 28.692 | 0.59 | 0.3 | | 0.6 | 0.27 | 44.4 | 0.635 | 0.31 |
| | | P2 | | | | | | | | | |
| | | TT | | | | | | | | | |
| 12040 (Jimai No. 2) | ZM009126 | CC | 45.276 | 0.683333 | 0.36 | 41.85 | 0.703333 | 0.34 | | 0.675 | 0.335 |
| | | P2 | | | | | | | | | |
| | | TT | | | | | | | | | |
| Neixiang No. 5 | ZM009523 | CC | 45.52 | 0.716667 | 0.32 | 44.255 | 0.733333 | 0.33 | 55.68 | 0.705 | 0.35 |
| | | P2 | | | | | | | | | |
| | | TT | | | | | | | | | |
| Zhengzhou No. 6 | ZM009463 | CC | 42.91 | 0.706667 | 0.35 | 38.225 | 0.676667 | 0.34 | 41.62 | 0.68 | 0.34 |
| | | P2 | | | | | | | | | |
| | | TT | | | | | | | | | |
| Aifeng No. 3 | ZM009603 | CC | 34.464 | 0.603333 | 0.31 | 32.37 | 0.593333 | 0.29 | 35.46 | 0.605 | 0.325 |
| | | P2 | | | | | | | | | |
| | | TT | | | | | | | | | |
| Baimangmai | ZM000215 | CC | 33.548 | 0.633333 | 0.3 | 28.74 | 0.6 | 0.29 | 32.5 | 0.625 | 0.29 |
| | | P2 | | | | | | | | | |
| | | TT | | | | | | | | | |
| Huangguaxian | ZM003050 | CC | 33.058 | 0.663333 | 0.29 | 23.65 | 0.613333 | 0.24 | | 0.645 | 0.205 |
| | | P2 | | | | | | | | | |
| | | TT | | | | | | | | | |
| Banjiemang | ZM002569 | CC | 28.61 | 0.606667 | 0.33 | 26.615 | 0.59 | 0.31 | | 0.58 | 0.265 |
| | | P2 | | | | | | | | | |
| | | TT | | | | | | | | | |
| Laolaixia | ZM001912 | CC | 34.6 | 0.606667 | 0.32 | 31.565 | 0.616667 | 0.31 | 31.56 | 0.61 | 0.305 |
| | | P2 | | | | | | | | | |
| | | TT | | | | | | | | | |
| Louguding | ZM001674 | CC | 30.522 | 0.586667 | 0.32 | 28.165 | 0.576667 | 0.3 | 31.94 | 0.6 | 0.31 |
| | | P2 | | | | | | | | | |
| | | TT | | | | | | | | | |
| Xishanbiansui | ZM001846 | CC | 32.284 | 0.586667 | 0.32 | | | | 31.32 | 0.58 | 0.305 |
| | | P2 | | | | | | | | | |
| | | TT | | | | | | | | | |
| Honggoudou | ZM002681 | CC | 33.116 | 0.55 | 0.32 | 29 | 0.55 | 0.31 | 34.34 | 0.535 | 0.325 |
| | | P2 | | | | | | | | | |
| | | TT | | | | | | | | | |
| Baihuomai | ZM001499 | CC | 28.634 | 0.546667 | 0.29 | 26.075 | 0.543333 | 0.3 | | 0.545 | 0.275 |
| | | P2 | | | | | | | | | |
| | | TT | | | | | | | | | |
| Sanyuehuang | ZM002685 | CC | 27.68 | 0.563333 | 0.3 | 21.75 | 0.55 | 0.26 | 25.18 | 0.565 | 0.285 |
| | | P2 | | | | | | | | | |
| | | TT | | | | | | | | | |
| Hongqiangchang | ZM003747 | CC | 28.636 | 0.573333 | 0.3 | 22.325 | 0.556667 | 0.28 | 25.24 | 0.575 | 0.275 |
| | | P2 | | | | | | | | | |
| | | TT | | | | | | | | | |
| Youzimai | ZM002668 | CC | 34.396 | 0.593333 | 0.32 | 27.33 | 0.573333 | 0.3 | 30.32 | 0.595 | 0.285 |
| | | P2 | | | | | | | | | |
| | | TT | | | | | | | | | |
| Pingyuan 50 | ZM002974 | CC | 40.224 | 0.62 | 0.34 | 33.775 | 0.593333 | 0.32 | | 0.605 | 0.305 |
| | | P2 | | | | | | | | | |
| | | TT | | | | | | | | | |
| Baiqimai | ZM017630 | CC | 27.684 | 0.603333 | 0.31 | | | | | 0.575 | 0.28 |
| | | P2 | | | | | | | | | |
| | | TT | | | | | | | | | |
| Baituzitou | ZM002330 | CC | 29.43 | 0.556667 | 0.31 | 25.99 | 0.563333 | 0.31 | | 0.545 | 0.265 |
| | | P2 | | | | | | | | | |
| | | TT | | | | | | | | | |
| Youmangsaogudan | ZM002659 | CC | 29.514 | 0.553333 | 0.33 | 27.01 | 0.59 | 0.3 | 28.84 | 0.56 | 0.3 |
| | | P2 | | | | | | | | | |
| | | TT | | | | | | | | | |
| Fuyanghong | ZM011007 | CC | 30.854 | 0.603333 | 0.31 | | | | 30.54 | 0.605 | 0.295 |
| | | P2 | | | | | | | | | |
| | | TT | | | | | | | | | |
| Mazhamai | ZM003807 | CC | 30.03 | 0.57 | 25.94 | 0.563333 | | 0.3 | 32.14 | 0.565 | 0.35 |
| | | P2 | | | | | | | | | |
| | | TT | | | | | | | | | |
| Qingchangmai | ZM003793 | CC | 24.166 | 0.556667 | 0.3 | 23.775 | 0.58 | 0.27 | 25.66 | 0.58 | 0.285 |
| | | P2 | | | | | | | | | |
| | | TT | | | | | | | | | |
| Huomai | ZM020632 | CC | 22.954 | 0.553333 | 0.29 | | | | | 0.585 | 0.26 |
| | | P2 | | | | | | | | | |
| | | TT | | | | | | | | | |
| Meiqianwu | ZM006160 | CC | 29.99 | 0.636667 | 0.3 | 28.6 | 0.596667 | 0.29 | | 0.635 | 0.3 |
| | | P2 | | | | | | | | | |
| | | TT | | | | | | | | | |
| Jianmai | ZM003080 | CC | 30.926 | 0.613333 | 0.31 | 32.425 | 0.623333 | 0.32 | | 0.635 | 0.255 |
| | | P2 | | | | | | | | | |
| | | TT | | | | | | | | | |
| Sanyuehuang | ZM011120 | CC | 31.022 | 0.606667 | 0.32 | 28.71 | 0.593333 | 0.28 | 29.64 | 0.595 | 0.285 |
| | | P2 | | | | | | | | | |
| | | TT | | | | | | | | | |
| Xiaofoshou | ZM002686 | CC | 28.31 | 0.56 | 0.3 | 26.33 | 0.54 | 0.31 | 29.72 | 0.58 | 0.315 |
| | | P2 | | | | | | | | | |
| | | TT | | | | | | | | | |
| Hongheshangtou | ZM003393 | CC | 35.58 | 0.59 | 0.32 | 31.03 | 0.61 | 0.31 | | 0.595 | 0.28 |
| | | P2 | | | | | | | | | |
| | | TT | | | | | | | | | |
| Dakoumai | ZM003131 | CC | 30.756 | 0.593333 | 0.31 | 26.905 | 0.563333 | 0.29 | | 0.575 | 0.265 |
| | | P2 | | | | | | | | | |
| | | TT | | | | | | | | | |
| Tumangmai | ZM004154 | CC | 32.884 | 0.586667 | 0.31 | 28.855 | 0.586667 | 0.3 | | 0.55 | 0.275 |
| | | P2 | | | | | | | | | |
| | | TT | | | | | | | | | |
| Baitiaoyu | ZM003069 | CC | 28.606 | 0.573333 | 0.3 | 25.07 | | | | 0.58 | 0.295 |
| | | P2 | | | | | | | | | |
| | | TT | | | | | | | | | |
| Baimangmai | ZM003650 | CC | 26.98 | 0.59 | 0.29 | 28.685 | 0.6 | 0.29 | 28.38 | 0.585 | 0.275 |
| | | P2 | | | | | | | | | |
| | | TT | | | | | | | | | |
| Dayuhua | ZM006348 | CC | 30.738 | 0.566667 | 0.31 | 27.885 | 0.56 | 0.3 | | 0.59 | 0.305 |
| | | P2 | | | | | | | | | |
| | | TT | | | | | | | | | |
| Fumai | ZM003145 | CC | 34.164 | 0.613333 | 0.31 | 28.245 | 0.59333 | 0.29 | | 0.64 | 0.29 |
| | | P2 | | | | | | | | | |
| | | TT | | | | | | | | | |
| Laoqimai | ZM003663 | CC | 33.552 | 0.66 | 0.34 | 29.225 | 0.613333 | 0.3 | 34.82 | 0.62 | 0.33 |
| | | P2 | | | | | | | | | |
| | | TT | | | | | | | | | |
| Chushanbao | ZM003138 | CC | 37.904 | 0.63 | 0.34 | | | | | 0.615 | 0.305 |
| | | P2 | | | | | | | | | |
| | | TT | | | | | | | | | |
| Dalibanmang | ZM001742 | CC | 38.622 | 0.626667 | 0.34 | 41.05 | 0.656667 | 0.35 | 41.78 | 0.625 | 0.34 |
| | | P2 | | | | | | | | | |
| | | TT | | | | | | | | | |
| Liuyuehuang | ZM005141 | CC | 41.752 | 0.74 | 0.32 | 30.59 | 0.723333 | 0.33 | 38 | 0.705 | 0.605 |
| | | P2 | | | | | | | | | |
| | | TT | | | | | | | | | |
| Gejiaxiang | ZM012971 | CC | 41.702 | 0.736667 | 0.32 | 37.38 | 0.736667 | 0.31 | 49.5 | 0.765 | 0.325 |
| | | P2 | | | | | | | | | |
| | | TT | | | | | | | | | |
| Dachunbaisilengmai 2 | ZM011525 | CC | 40.936 | 0.723333 | 0.31 | 37.695 | 0.726667 | 0.31 | 42.52 | 0.715 | 0.325 |
| | | P2 | | | | | | | | | |
| | | TT | | | | | | | | | |
| Bailanghuimai | ZM018849 | CC | 37.18 | 0.676667 | 0.32 | 37.7 | 0.696667 | 0.32 | 38.56 | 0.695 | 0.37 |
| | | P2 | | | | | | | | | |
| | | TT | | | | | | | | | |
| Bendilhuanghuamai | ZM011565 | CC | 36.292 | 0.64 | 0.34 | 40.915 | 0.65 | 0.34 | 31.2 | 0.635 | 0.33 |
| | | P2 | | | | | | | | | |
| | | TT | | | | | | | | | |
| Zhahong | ZM018528 | CC | 37.982 | 0.71333 | 0.33 | 39.765 | 0.703333 | 0.31 | 36.3 | 0.68 | 0.315 |
| | | P2 | | | | | | | | | |
| | | TT | | | | | | | | | |
| Motuo wheat | ZM019907 | CC | 27.44 | 0.633333 | 0.3 | | 0.636667 | 0.29 | 25.68 | 0.625 | 0.275 |
| | | P2 | | | | | | | | | |
| | | TT | | | | | | | | | |
| Bianbachunmai-6 | ZM018930 | CC | 29.752 | 0.646667 | 0.3 | 31.875 | 0.653333 | 0.29 | 26.824 | 0.625 | 0.27 |
| | | P2 | | | | | | | | | |
| | | TT | | | | | | | | | |
| Baimang wheat | ZM008341 | CC | 30.708 | 0.61 | 0.3 | 26.88 | 0.596667 | 0.27 | 27.42 | 0.62 | 0.28 |
| | | P2 | | | | | | | | | |
| | | TT | | | | | | | | | |
| Wujiangzhuo | ZM020305 | CC | 39.61 | 0.726667 | 0.32 | 33.51 | 0.733333 | 0.3 | 43.4 | 0.75 | 0.33 |
| | | P2 | | | | | | | | | |
| | | TT | | | | | | | | | |
| Muzongzhuoga | ZM018569 | CC | 32.164 | 0.6 | 0.32 | 30.66 | 0.606667 | 0.29 | 31.04 | 0.595 | 0.31 |
| | | P2 | | | | | | | | | |
| | | TT | | | | | | | | | |
| Kangding wheat | ZM008347 | CC | 40.994 | 0.626667 | 0.33 | 38.65 | 0.61 | 0.31 | 39.5 | 0.59 | 0.355 |
| | | P2 | | | | | | | | | |
| | | TT | | | | | | | | | |
| Rikaze No. 54 | ZM010591 | CC | 39.87 | 0.68 | 0.31 | 32.65 | 0.68 | 0.29 | 40.98 | 0.695 | 0.345 |
| | | P2 | | | | | | | | | |
| | | TT | | | | | | | | | |
| Shanmai | ZM020774 | CC | 36.138 | 0.68 | 0.31 | 36.28 | 0.63 | 0.28 | 38.36 | 0.675 | 0.605 |
| | | P2 | | | | | | | | | |
| | | TT | | | | | | | | | |
| Yizhimai | ZM004779 | CC | 37.226 | 0.67 | 0.32 | 41.27 | 0.686667 | 0.29 | | 0.675 | 0.29 |
| | | P2 | | | | | | | | | |
| | | TT | | | | | | | | | |
| Dabaimai | ZM012760 | CC | 38.965 | 0.683333 | 0.34 | 38.03 | 0.71 | 0.31 | | 0.725 | 0.29 |
| | | P2 | | | | | | | | | |
| | | TT | | | | | | | | | |
| Galaohan | ZM005105 | CC | 37.453 | 0.70333 | 0.31 | 37.85 | 0.72 | 0.3 | 38.86 | 0.72 | 0.295 |
| | | P2 | | | | | | | | | |
| | | TT | | | | | | | | | |
| Huoliyan | ZM012793 | CC | 39.079 | 0.696667 | 0.31 | 37.555 | 0.68 | 0.28 | | 0.69 | 0.28 |
| | | P2 | | | | | | | | | |
| | | TT | | | | | | | | | |
| Shanmai | ZM020770 | CC | 36.682 | 0.76 | 0.3 | 23.135 | | | 35.26 | 0.705 | 0.31 |
| | | P2 | | | | | | | | | |
| | | TT | | | | | | | | | |
| Hongtuzi | ZM020815 | CC | 31.918 | 0.686667 | 0.32 | 29.225 | 0.673333 | 0.31 | 39.98 | 0.645 | 0.285 |
| | | P2 | | | | | | | | | |
| | | TT | | | | | | | | | |
| Baidatou | ZM004862 | CC | 39.268 | 0.613333 | 0.34 | 47.505 | 0.606667 | 0.33 | | | |
| | | P2 | | | | | | | | | |
| | | TT | | | | | | | | | |
| Jinhuangmai | ZM004780 | CC | 42.294 | 0.70333 | 0.34 | 28.91 | 0.72 | 0.33 | | 0.71 | 0.305 |
| | | P2 | | | | | | | | | |
| | | TT | | | | | | | | | |
| Baimazha | ZM020808 | CC | 31.126 | 0.576667 | 0.32 | 29.245 | 0.59 | 0.28 | 31.6 | 0.595 | 0.32 |
| | | P2 | | | | | | | | | |
| | | TT | | | | | | | | | |
| Laotutou | ZM004670 | CC | 32.752 | 0.666667 | 0.31 | 27.184 | 0.75 | 0.27 | 33.4 | 0.615 | 0.26 |
| | | P2 | | | | | | | | | |
| | | TT | | | | | | | | | |
| Huining No. 10 | ZM017313 | CC | 42.66 | 0.66 | 0.34 | 36.475 | 0.683333 | 0.31 | 47.54 | 0.735 | 0.33 |
| | | P2 | | | | | | | | | |
| | | TT | | | | | | | | | |
| Fan 6 | ZM010450 | CC | 38.66 | 0.63 | 0.35 | 38.415 | 0.623333 | 0.34 | 36.92 | 0.6 | 0.33 |
| | | P2 | | | | | | | | | |
| | | TT | | | | | | | | | |
| Tongjiaba wheat | ZM007916 | CC | 29.916 | 0.64 | 0.31 | 32.88 | 0.646667 | 0.31 | 35.02 | 0.625 | 0.305 |
| | | P2 | | | | | | | | | |
| | | TT | | | | | | | | | |
| Honghuamai | ZM007925 | CC | 32.86 | 0.646667 | 0.36 | 31.135 | 0.646667 | 0.32 | | 0.63 | 0.295 |
| | | P2 | | | | | | | | | |
| | | TT | | | | | | | | | |
| Baimaizi | ZM008547 | CC | 25.444 | 0.603333 | 0.32 | 26.425 | 0.6 | 0.3 | 29.3 | 0.555 | 0.33 |
| | | P2 | | | | | | | | | |
| | | TT | | | | | | | | | |
| Chengdu guangtou | ZM008365 | CC | 31.698 | 0.643333 | 0.32 | 28.015 | 0.62 | 0.3 | 33.6 | 0.64 | 0.335 |
| | | P2 | | | | | | | | | |
| | | TT | | | | | | | | | |
| Baihuamai | ZM008598 | CC | 23.704 | 0.563333 | 0.28 | 19.38 | 0.556667 | 0.27 | 24.04 | 0585 | 0.27 |
| | | P2 | | | | | | | | | |
| | | TT | | | | | | | | | |
| Huanxiangguo | ZM008249 | CC | 34.84 | 0.613333 | 0.31 | 31.11 | 0.613333 | 0.31 | 33.5 | 0.6 | 0.305 |
| | | P2 | | | | | | | | | |
| | | TT | | | | | | | | | |
| Hanzhongbai | ZM004029 | CC | 31.29 | 0.63 | 0.32 | 30.595 | 0.643333 | 0.33 | 32.52 | 0.575 | 0.305 |
| | | P2 | | | | | | | | | |
| | | TT | | | | | | | | | |
| Xiaosanyuehuang | ZM012165 | CC | 31.93 | 0.613333 | 0.31 | 33.18 | 0.63 | 0.31 | 38.34 | 0.645 | 0.32 |
| | | P2 | | | | | | | | | |
| | | TT | | | | | | | | | |
| Suotiaohongmai | ZM012711 | CC | 30.642 | 0.616667 | 0.3 | 31.455 | 0.646667 | 0.31 | 32.62 | 0.655 | 0.295 |
| | | P2 | | | | | | | | | |
| | | TT | | | | | | | | | |
| Hongxumai | ZM012545 | CC | 32.878 | 0.623333 | 0.31 | 32.63 | 0.65 | 0.31 | 33.28 | 0.67 | 0.315 |
| | | P2 | | | | | | | | | |
| | | TT | | | | | | | | | |
| Zipi | ZM011741 | CC | 26.502 | 0.61 | 0.26 | 21.395 | 0.623333 | 0.23 | 25.468 | 0.655 | 0.26 |
| | | P2 | | | | | | | | | |
| | | TT | | | | | | | | | |
| Baimangmai | ZM008732 | CC | 26.842 | 0.56 | 0.3 | 21.42 | 0.55 | 0.26 | 26.56 | 0.575 | 0.29 |
| | | P2 | | | | | | | | | |
| | | TT | | | | | | | | | |
| Yangmai | ZM012030 | CC | 27.73 | 0.586667 | 0.29 | 23.325 | 0.606667 | 0.27 | 29.02 | 0.62 | 0.285 |
| | | P2 | | | | | | | | | |
| | | TT | | | | | | | | | |
| Zhushimai | ZM008809 | CC | 33.664 | 0.66 | 0.3 | 28.83 | 0.643333 | 0.28 | 32.34 | 0.645 | 0.27 |
| | | P2 | | | | | | | | | |
| | | TT | | | | | | | | | |
| Biantouguangkemai | ZM012032 | CC | 25.848 | 0.573333 | 0.31 | 25.85 | 0.59 | 0.3 | 27.99 | 0.605 | 0.305 |
| | | P2 | | | | | | | | | |
| | | TT | | | | | | | | | |
| Changmangshibiantou | ZM011859 | CC | 24.094 | 0.573333 | 0.28 | 22.385 | 0.606667 | 0.26 | 30.76 | 0.605 | 0.285 |
| | | P2 | | | | | | | | | |
| | | TT | | | | | | | | | |
| Zhugoumai | ZM012061 | CC | 31.776 | 0.643333 | 0.3 | 30.025 | 0.663333 | 0.28 | 33.26 | 0.645 | 0.295 |
| | | P2 | | | | | | | | | |
| | | TT | | | | | | | | | |
| Dianxihongkeyangmai | ZM012096 | CC | 36.996 | 0.653333 | 0.32 | | 0.68 | 0.3 | 39.92 | 0.655 | 0.31 |
| | | P2 | | | | | | | | | |
| | | TT | | | | | | | | | |
| Baidongmai | ZM005439 | CC | 25.724 | 0.593333 | 0.3 | 29.07 | 0.61 | 0.31 | 23.78 | 0.625 | 0.27 |
| | | P2 | | | | | | | | | |
| | | TT | | | | | | | | | |
| Hongchunmai | ZM005294 | CC | 41.998 | 0.736667 | 0.29 | 43.505 | 0.74 | 0.31 | 37.7 | 0.76 | 0.275 |
| | | P2 | | | | | | | | | |
| | | TT | | | | | | | | | |
| Chunmai | ZM013048 | CC | 36.618 | 0.696667 | 0.31 | | | | | 0.67 | 0.3 |
| | | P2 | | | | | | | | | |
| | | TT | | | | | | | | | |
| Hongdongmai | ZM005241 | CC | 32.712 | 0.69 | 0.31 | 32.325 | 0.696667 | 0.29 | | 0.705 | 0.26 |
| | | P2 | | | | | | | | | |
| | | TT | | | | | | | | | |
| Hongchunmai | ZM005317 | CC | 34.068 | 0.64 | 0.31 | 39.36 | 0.683333 | 0.32 | | 0.645 | 0.28 |
| | | P2 | | | | | | | | | |
| | | TT | | | | | | | | | |
| Hongjinbaoyin | ZM013034 | CC | 49.944 | 0.73 | 0.34 | 44.72 | 0.74 | 0.33 | | 0.695 | 0.325 |
| | | P2 | | | | | | | | | |
| | | TT | | | | | | | | | |
| Hongdongmai | ZM005176 | CC | 27.096 | 0.706667 | 0.31 | 30.725 | 0.696667 | 0.28 | | 0.695 | 0.255 |
| | | P2 | | | | | | | | | |
| | | TT | | | | | | | | | |
| Wumangchunmai | ZZ005330 | CC | 33.494 | 0.643333 | 0.31 | 36.7 | 0.683333 | 0.3 | | 0.64 | 0.3 |
| | | P2 | | | | | | | | | |
| | | TT | | | | | | | | | |
| Kashi No. 1 | H02027 | CC | 36.364 | 0.676667 | 0.33 | | | | | 0.66 | 0.305 |
| | | P2 | | | | | | | | | |
| | | TT | | | | | | | | | |
| Kashi Baipi | ZM010128 | CC | 43.326 | 0.723333 | 0.32 | 33.8 | 0.73 | 0.3 | 42.92 | 0.72 | 0.345 |
| | | P2 | | | | | | | | | |
| | | TT | | | | | | | | | |
| Chinese Spring | ZM005452 | CC | 29.336 | 0.596667 | 0.31 | 25.065 | 0.573333 | 0.29 | 25.62 | 0.58 | 0.33 |
| | | P2 | | | | | | | | | |
| | | TT | | | | | | | | | |

| | | | | | | | | | |
|---|---|---|---|---|---|---|---|---|---|
| Average value | 33.4308 | 0.6330 | 0.3119 | 30.7148 | 0.6306 | 0.2960 | 34.2130 | 0.6260 | 0.2992 |
| Accuracy | 109/171 | 109/170 | | 120/153 | 100/156 | | 78/126 | 120/168 | |

### Example 5 Identification of SNP 488 and SNP 2144 and Design of Specific Primer Sets

I. Exploration of Specific SNPs

Wheat lines for testing: 34 wheat lines which distributed over different wheat regions of China with greatly different grain traits (No. C1-34, see Table 21 for specific information on materials) were selected as materials for exploring polymorphic site.

2. Sequence Alignment

Each wheat line for testing was subjected to the following steps:

1. Genomic DNA from wheat materials for testing was extracted.

2. Using the genomic DNA extracted from step 1 as template, a primer set composed of TaTPP-F1 and TaTPP-R1 was used for PCR amplification, giving a PCR amplification product.

TaTPP-F1 (SEQ ID NO: 4):
5′-CGTGTGGTTGTTTGCGTG-3′;
TaTPP-R1 (SEQ ID NO: 5):
5′-CTAGATATAGGCGAGGGTTATTAC-3′.

3. The PCR amplification product obtained from step 2 was subjected to cloning and sequencing. 24 clones were sequenced for each wheat line.

4. The sequences were assembled and aligned.

The sequencing results of 24 clones of each wheat material were subjected to genome A sequence assembly and alignment analysis. Two PCR amplification products for genome A from different wheat lines were obtained. The two PCR amplification products were both 2254 bp in length, both have 5′ terminal being consistent with TaTPP-F1, and 3′ terminal being reverse complementary to TaTPP-R1, butt one PCR amplification product comprised the nucleotides at positions 467-514 from 5′ terminal, as shown by SEQ ID NO:19, and the other PCR amplification product comprised the nucleotides at positions 467-514 from the 5 end as shown by SEQ ID NO:20. A similar result was observed when analyzing positions 2121-2168. One PCR amplification product comprised the nucleotides at positions 2121-2168 from the 5′ end, as shown by SEQ ID NO:25, and the other PCR amplification product comprised the nucleotides at positions 2121-2168 from the 5 end as shown by SEQ ID NO:26.

Based on the sequence alignment of PCR amplification products from all tested wheat lines, one SNP was discovered and designated as 488 SNP, with A/C polymorphism, and another SNP was discover and designated as 2144 SNP with A/T polymorphism. The 488 SNP corresponded to the nucleotide at position 22 from 5′ end of SEQ ID NO:24, and the 2144 SNP corresponded to the nucleotide at position 30 from the 5′ end of SEQ ID NO: 30.

The 488 SNP-based genotype and 2144 SNP based genotype of each tested wheat line is shown in Table 1.

II. Design of Specific Primer Sets

Based on the specific SNPs as described above, the following KASP-based primer sets were designed:

488F1 (SEQ ID NO: 21):
5′-GAAGGTGACCAAGTTCATGCTGGTCGTGTTCCTGGACTACGAC-3′;
488F2 (SEQ ID NO: 22):
5′-GAAGGTCGGAGTCAACGGATTGGTCGTGTTCCTGGACTACGAA-3′;
488C (SEQ ID NO: 23):
5′-TCGGCGACGATGGGCGAGAGCGT-3′

Based on the specific SNPs as described above, the following KASP-based primer sets were designed:

2144F1 (SEQ ID NO: 27):
5′-GAAGGTGACCAAGTTCATGCTTCACAGACTGCCACATCAGCGGC
T-3′;
2144F2 (SEQ ID NO: 28):
5′-GAAGGTCGGAGTCAACGGATTTCACAGACTGCCACATCAGCGGC
A-3′;
2144C (SEQ ID NO: 29):
5′-TCTTGATAAATCAGTGCCAGGAG-3′;

III. Use of the Specific Primer Sets for Analyzing a Larger Collection of Wheat Lines.

The primers were used to analyze the different wheat varieties of Tables 3, 4 and 5 and the results are summarized therein.

The results for tested wheat materials of AA genotype of SNP 488 or AA genotype for SNP 2144 were as shown in Table 3 (including the results for each tested wheat, and the average value for all the tested wheat having said genotype). The results for tested wheat materials of A/C genotype for SNP 488 or A/T genotype for SNP 2144 were as shown in Table 4 (including the results for each tested wheat, and the average value for all the tested wheat having said genotype). The results for tested wheat materials of CC genotype for SNP 488 or TT genotype for SNP 2144 were as shown in Table 5 (including the results for each tested wheat, and the average value for all the tested wheat having said genotype). From the general trend, the wheat of the AA genotype for SNP 488 or AA genotype for SNP 2144 had a heavier thousand-kernel weight than the wheat of CC genotype for SNP 488 or TT genotype for SNP 2144, and the wheat of AA genotype for SNP 488 or AA genotype for SNP 2144 had a longer kernel length than the wheat of CC genotype for SNP 488 or TT genotype for SNP 2144.

IV. Correlation Analysis for SNP 488

For the tested wheat materials, the correlation in varieties for breeding was analyzed, with the results being shown in Table 6. According to the results, the three-year average thousand-kernel weight was 41.50 g for tested wheat of AA genotype, and 36.45 g for tested wheat of CC genotype, showing a remarkably significant difference (P<0.01); with regard to the kernel length trait, the material of wheat of AA genotype had a longer kernel length than the material of wheat of CC genotype, showing a significant or remarkably significant difference (P<0.05 or P<0.01). As can be seen, compared with the CC genotype, the AA genotype is a genotype with excellent grain traits.

TABLE 6

| Varieties for | 2002 | | | 2005 | | | 2006 | | |
|---|---|---|---|---|---|---|---|---|---|
| breeding | AA | CC | P | AA | CC | P | AA | CC | P |
| thousand-kernel | 42.39 ± 5.74 | 38.50 ± 6.97 | 0.018* | 39.87 ± 5.46 | 33.84 ± 5.93 | 0.000** | 42.25 ± 5.58 | 37.03 ± 5.55 | 0.000** |
| weight (g) | | | | | | | | | |
| kernel length | 0.68 ± 0.054 | 0.64 ± 0.05 | 0.002** | 0.68 ± 0.04 | 0.64 ± 0.04 | 0.000** | 0.69 ± 0.04 | 0.65 ± 0.04 | 0.000** |
| (mm) | | | | | | | | | |
| kernel width | 0.33 ± 0.02 | 0.32 ± 0.03 | 0.053 | 0.32 ± 0.02 | 0.31 ± 0.02 | 0.000** | 0.33 ± 0.02 | 0.32 ± 0.02 | 0.009** |
| (mm) | | | | | | | | | |

Note:
*P < 0.05, **P < 0.01.

For the tested wheat materials, the correlation in local varieties was analyzed, with the results being shown in Table 7. According to the results, the three-year average thousand kernel weight was 38.9 g for tested wheat of AA genotype, and 31.55 g for tested wheat of CC genotype, showing a remarkably significant difference (P<0.01); with regard to the kernel length trait, the material of wheat of AA genotype had a longer kernel length than the wheat material of CC genotype, showing a significant or remarkably significant difference (P<0.05 or P<0.01). As can be seen, compared with the CC genotype, the AA genotype is a genotype with excellent grain traits.

TABLE 7

| | 2002 | | | 2005 | | | 2006 | | |
|---|---|---|---|---|---|---|---|---|---|
| Local varieties | AA | CC | P | AA | CC | P | AA | CC | P |
| thousand-kernel | 40.94 ± 8.71 | 32.57 ± 5.00 | 0.000** | 36.56 ± 7.35 | 29.68 ± 6.29 | 0.001** | 39.15 ± 7.30 | 32.40 ± 4.80 | 0.000** |
| weight (g) | | | | | | | | | |
| kernel length | 0.68 ± 0.60 | 0.62 ± 0.06 | 0.010** | 0.67 ± 0.07 | 0.63 ± 0.05 | 0.009** | 0.68 ± 0.06 | 0.63 ± 0.05 | 0.001** |
| (mm) | | | | | | | | | |
| kernel width | 0.31 ± 0.03 | 0.30 ± 0.03 | 0.258 | 0.31 ± 0.02 | 0.29 ± 0.02 | 0.020 | 0.32 ± 0.02 | 0.31 ± 0.02 | 0.065 |
| (mm) | | | | | | | | | |

Note:
*P < 0.05, **P < 0.01.

V. Correlation Analysis for SNP 2144

A similar analysis was conducted for SNP 2144

| | 2002 | | | 2005 | | | 2006 | | |
|---|---|---|---|---|---|---|---|---|---|
| Local varieties | AA | TT | P | AA | TT | P | AA | TT | P |
| thousand-kernel | 42.39 ± 5.74 | 38.50 ± 6.97 | 0.018* | 39.87 ± 5.46 | 33.84 ± 5.93 | 0.000** | 42.25 ± 5.58 | 37.03 ± 5.55 | 0.000** |
| weight (g)) | | | | | | | | | |
| kernel length | 0.68 ± 0.054 | 0.64 ± 0.05 | 0.002** | 0.68 ± 0.04 | 0.64 ± 0.04 | 0.000** | 0.69 ± 0.04 | 0.65 ± 0.04 | 0.000** |
| (mm) | | | | | | | | | |
| kernel width | 0.33 ± 0.02 | 0.32 ± 0.03 | 0.053 | 0.32 ± 0.02 | 0.31 ± 0.02 | 0.000** | 0.33 ± 0.02 | 0.32 ± 0.02 | 0.009** |
| (mm) | | | | | | | | | |

Note:
*P < 0.05, **P < 0.01.

VI. Correlation Analysis for P 1/P2 Promoters

A similar analysis was conducted for P 1/P2

| | 2002 | | | 2005 | | | 2006 | | |
|---|---|---|---|---|---|---|---|---|---|
| Local varieties | P1 | P2 | P | P1 | P2 | P | P1 | P2 | P |
| thousand-kernel | 42.39 ± 5.74 | 38.50 ± 6.97 | 0.018* | 39.87 ± 5.46 | 33.84 ± 5.93 | 0.000** | 42.25 ± 5.58 | 37.03 ± 5.55 | 0.000** |
| weight (g)) | | | | | | | | | |
| kernel length | 0.68 ± 0.054 | 0.64 ± 0.05 | 0.002** | 0.68 ± 0.04 | 0.64 ± 0.04 | 0.000** | 0.69 ± 0.04 | 0.65 ± 0.04 | 0.000** |
| (mm) | | | | | | | | | |
| kernel width | 0.33 ± 0.02 | 0.32 ± 0.03 | 0.053 | 0.32 ± 0.02 | 0.31 ± 0.02 | 0.000** | 0.33 ± 0.02 | 0.32 ± 0.02 | 0.009** |
| (mm) | | | | | | | | | |

Note:
*P < 0.05, **P < 0.01.

### Example 6 Identification of Different Haplotypes Based on SNPs in TaTPP-7A Promoter Region and Coding Sequence

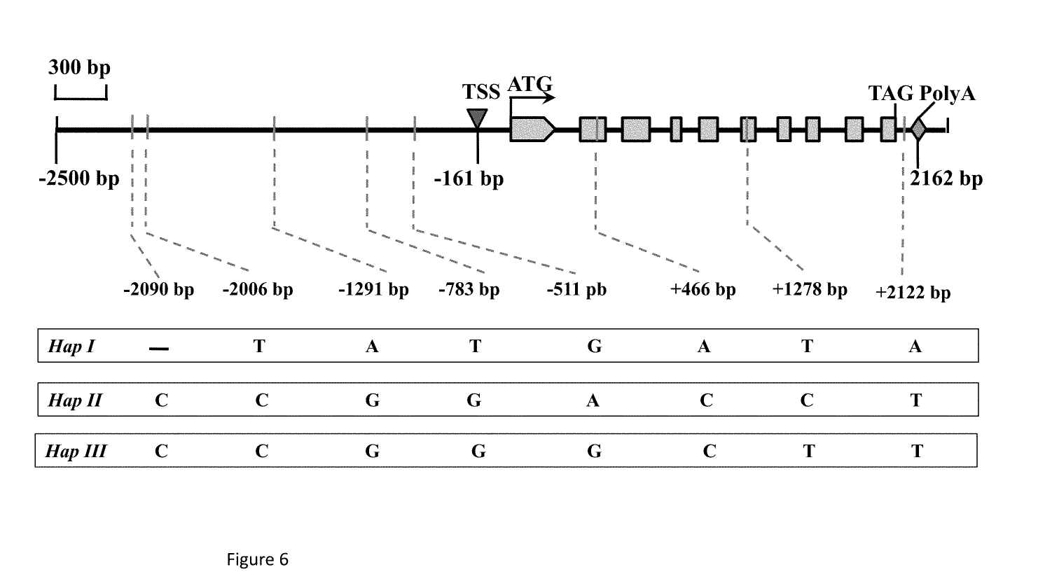

FIG. 6 summarizes the different haplotypes for SNPs found in the TaTPP-7A promoter region and coding sequence which could be identified when analyzing a large panel of wheat varieties.

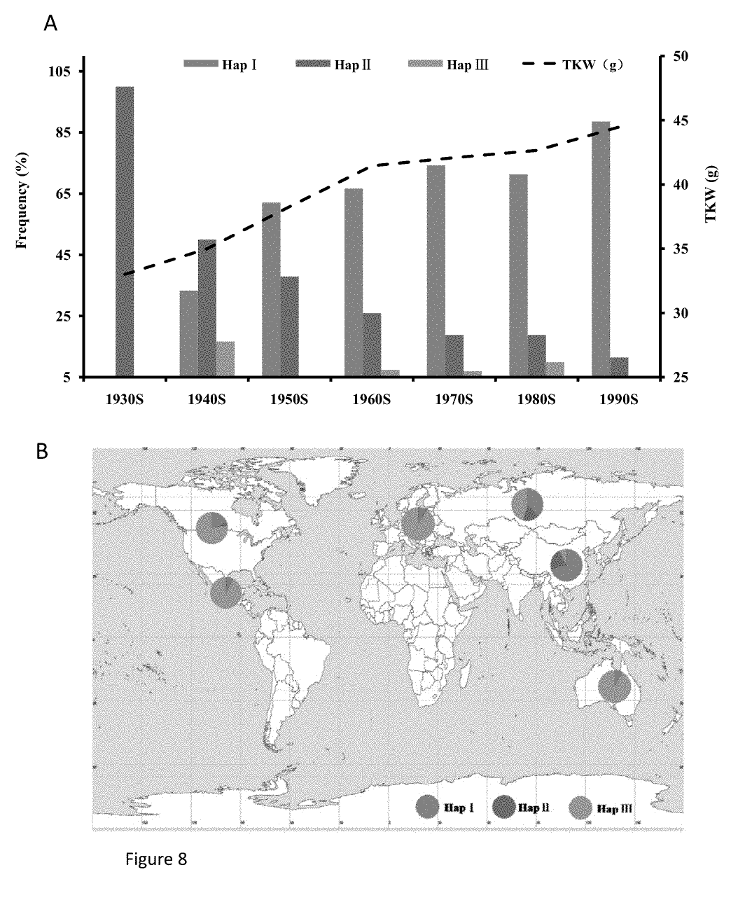

Haplotype I (Hap I) represents the following alleles for the different SNPs
SNP 409/410: TG
SNP 493 T
SNP 1208: A
SNP 1708: T
SNP 1980: G
SNP 488: A
SNP 1300: T
SNP 2144: A
   Haplotype II (Hap II) represents the following alleles for the different SNPs
SNP 409/410: TCG
SNP 493 C
SNP 1208: G
SNP 1708: G
SNP 1980: A
SNP 488: C
SNP 1300: C
SNP 2144: T
   Haplotype III (Hap III) represents the following alleles for the different SNPs
SNP 409/410: TCG
SNP 493 C
SNP 1208: G
SNP 1708: G
SNP 1980: G
SNP 488: C
SNP 1300: T
SNP 2144: T
   FIG. 8 a indicates the relative occurrence of the haplotypes in Chinese wheat varieties over time. Whereas in the 1930s all Chinese varieties analyzed had Hap II haplotype (middle bar), from the 1940s on, the relative occurrence of Hap I haplotype increased steadily (left bar) while HapII (middle bar) and Hap III occurrence gradually decreased. This correlated with the increase in Thousand Kernel Weight (indicated by the dashed line) over time. FIG. 8 Panel B. represents the geographic distribution of the different Haplotypes. In China, the majority of the analyzed wheat lines exhibit Hap I haplotype. In the Russian Federation, the Hap I haplotype is also predominantly present, but Hap III presence is also significant, and even Hap II is represented. In North and Middle America, Europe and Australia, the predominant haplotype of the analyzed lines is Hap III, with only a minor relative occurrence of HapI.

1-26. (canceled)

27. A protein comprising an amino acid sequence having trehalose-6 phosphate phosphatase enzymatic activity, wherein the amino acid sequence is selected from the group consisting of:
a) an amino acid sequence of SEQ ID NO: 1;
b) an amino acid sequence having at least 90% sequence identity to the amino acid sequence of SEQ ID No: 1; and
c) an amino acid sequence of SEQ ID NO: 1 wherein one or more amino acid residues are substituted or deleted or inserted, and wherein the presence of the protein is associated with increased grain length, grain width or increased thousand kernel weight, wherein the Asp residue at position 112 is substituted by a Glu residue, and/or wherein the Ala residue at position 241 is substituted by a Val residue.

28. A nucleic acid molecule comprising a nucleotide sequence encoding the protein of claim 27.

29. The nucleic acid molecule of claim 28, wherein the nucleic acid molecule is selected from the group consisting of:
a) a nucleotide sequence of SEQ ID NO: 2;
b) a nucleotide sequence of SEQ ID NO: 3 from nucleotide positions 23 to nucleotide position 2115;
c) a nucleotide sequence of SEQ ID NO: 3
d) a nucleotide sequence which hybridizes with a DNA molecule according to any one of a to c above under stringent conditions, wherein the nucleotide sequence codes for a protein comprising an amino acid sequence having trehalose-6 phosphate phosphatase enzymatic activity, wherein the amino acid sequence is selected from the group consisting of:
i) an amino acid sequence of SEQ ID NO: 1;
ii) an amino acid sequence having at least 90% sequence identity to the amino acid sequence of SEQ ID No: 1; and
iii) an amino acid sequence of SEQ ID NO: 1 wherein one or more amino acid residues are substituted or deleted or inserted, and wherein the presence of the protein is associated with increased grain length, grain width or increased thousand kernel weight, wherein the Asp residue at position 112 is substituted by a Glu residue, and/or wherein the Ala residue at position 241 is substituted by a Val residue; and
e) a nucleotide sequence having at least 90% sequence identity to the nucleotide sequence of SEQ ID NO: 3 from nucleotide positions 23 to nucleotide position 2115 or the nucleotide sequence of SEQ ID NO: 2.

30. A recombinant expression cassette comprising the following operably linked DNA elements:
a) a plant-expressible promoter;
b) a DNA region encoding the protein of claim 27; and
c) a DNA region comprising a transcription termination and polyadenylation region.

31. A recombinant expression vector, transgenic cell line, transgenic plant tissue, transgenic plant or recombinant strain, or grain or seed comprising the nucleic acid molecule of claim 28.

32. The transgenic plant of claim 31, wherein the plant comprises a cereal plant.

33. A recombinant expression vector, transgenic cell line, transgenic plant tissue, transgenic plant or recombinant strain, or grain or seed comprising the recombinant expression cassette of claim 30.

34. A method of modulating plant grains by expressing the nucleic acid molecule of claim 28 in plants comprising the step of regulating:
a) size;
b) thousand-kernel weight;
c) kernel weight;
d) kernel length;
e) kernel width; and
f) kernel thickness.

35. A method of modulating plants by expressing the nucleic acid molecule of claim 28 in plants comprising the step of regulating:
a) tiller length;
b) spike length; and
c) grain yield.

36. A method of producing plants comprising the steps of:
a) increasing the level or activity of the protein of claim 27;
b) introducing into a plant cell or a plant, to obtain a transgenic plant, a recombinant expression cassette comprising the following operably linked DNA elements:
i) a plant-expressible promoter;
ii) a DNA region encoding the protein of claim 27; and
iii) a DNA region comprising a transcription termination and polyadenylation region
wherein, as compared to a starting plant or control plant, the plant comprises
1) an increased thousand-kernel weight in grains;
2) an increased kernel weight in grains;
3) a larger size in grains;
4) a longer kernel length in grains;
5) a wider kernel width in grains;
6) a thicker kernel thickness in grains;
7) an increased tiller length;
8) an increased spike length;
9) an increased grain number; and
10) an increased grain yield.

37. A method for increasing the content or activity of the nucleic acid of claim 28 in grains comprising increasing:
a) thousand-kernel weight;
b) kernel weight;
c) size;
d) length;
e) width; and
f) thickness.

38. A method for increasing the content or activity of the nucleic acid of claim 28 in plants comprising increasing:
a) tiller length;
b) spike length;
c) grain number; and
d) grain yield.

39. A method of plant breeding comprising expressing in a plant or cell thereof the protein of claim 27.

40. An isolated promoter region comprising:
a. a nucleotide sequence of SEQ ID NO:14 or SEQ ID NO: 15; and
b. a nucleotide sequence comprising at least 90%, 95% or 99% sequence identity thereto.

41. A recombinant gene comprising the following operably linked DNA fragments:
a. the promoter region of claim 40;
b. a DNA region encoding an RNA molecule or a protein of interest; and
c. a transcription termination and polyadenylation region functional in plant cells.

42. A plant comprising the recombinant gene of claim 41.

43. A method for identifying or assisting in identifying, wheat grain traits, comprising the step of:
detecting whether the genotype based on a 488 SNP site in the genomic DNA of the wheat to be tested is AA genotype, AC genotype or CC genotype;
wherein
the wheat of AA genotype has better grain traits than the wheat of CC genotype;
the better grain traits are higher thousand-kernel weight and/or longer kernel length;
the 488 SNP site refers to the nucleotide at position 22 from 5′ end of SEQ ID NO: 24.

44. A method for identifying or assisting in identifying the thousand-kernel weight of wheat grains, comprising the steps of:
detecting whether the genotype based on a 488 SNP site in the genomic DNA of the wheat to be tested is AA genotype, AC genotype or CC genotype;
wherein
if the genotype is AA genotype, the wheat to be tested is selected as a candidate for wheat of high thousand-kernel weight;
if the genotype is CC genotype, the wheat to be tested is selected as candidate for wheat of low thousand-kernel weight;
the wheat of high thousand-kernel weight refers to wheat whose grains have a thousand-kernel weight ≥35 g;
the wheat of low thousand-kernel weight refers to wheat whose grains have a thousand-kernel weight <35 g;
the 488 SNP site refers to the nucleotide at position 22 from 5′ terminal of SEQ ID NO:24.

45. A method for identifying or assisting in identifying the kernel length of wheat grains, comprising the steps of:
detecting whether the genotype based on a 488 SNP site in the genomic DNA of the wheat to be tested is AA genotype, AC genotype or CC genotype;
wherein
if the genotype is AA genotype, the wheat to be tested is selected as a candidate for wheat of long kernel length;
if the genotype is CC genotype, the wheat to be tested is selected as a candidate for wheat of short kernel length;
the wheat of long kernel length refers to wheat whose grains have a kernel length ≥0.65 mm;
the wheat of short kernel length refers to wheat whose grains have a kernel length <0.65 mm;
the 488 SNP site refers to the nucleotide at position 22 from 5′ terminal of SEQ ID NO:24.

46. A primer set I, selected from the group consisting of 488F1, 488F2 and 488C;
wherein the primer 488F1 comprises:
(b1) a single-stranded DNA molecule comprising SEQ ID NO:21; or
(b2) a DNA molecule obtained by subjecting SEQ ID NO: 21 to substitution or deletion or addition of one or several nucleotides and having the same function as SEQ ID NO:21;
wherein the primer 488F2 comprises:
(b3) a single-stranded DNA molecule comprising SEQ ID NO:22; or
(b4) a DNA molecule obtained by subjecting SEQ ID NO: 22 to substitution or deletion or addition of one or several nucleotides and having the same function as SEQ ID NO:22;
wherein the primer 488C comprises:
(b5) a single-stranded DNA molecule comprising SEQ ID NO:23; or
(b6) a DNA molecule obtained by subjecting SEQ ID NO:23 to substitution and/or deletion and/or addition of one or several nucleotides and having the same function as SEQ ID NO:23.

47. A method for identifying or assisting in identifying wheat grain traits, comprising the steps of:
detecting whether the genotype based on 2144 SNP site in the genomic DNA of the wheat to be tested is AA genotype, AT genotype or TT genotype;
wherein
the wheat of AA genotype has better grain traits than the wheat of TT genotype;
the better grain traits are shown as higher thousand-kernel weight and/or longer kernel length;
the 2144 SNP site refers to the nucleotide at position 24 from 5′ end of SEQ ID NO: 30.

48. A method for identifying or assisting in identifying the thousand-kernel weight of wheat grains, comprising the steps of:
detecting whether the genotype based on a 2144 SNP site in the genomic DNA of the wheat to be tested is AA genotype, AT genotype or TT genotype;
wherein
if the genotype is AA genotype, the wheat to be tested is selected as a candidate for wheat of high thousand-kernel weight;
if the genotype is TT genotype, the wheat to be tested is selected as a candidate for wheat of low thousand-kernel weight;
the wheat of high thousand-kernel weight refers to wheat whose grains have a thousand-kernel weight ≥35 g;
the wheat of low thousand-kernel weight refers to wheat whose grains have a thousand-kernel weight <35 g;
the 2144 SNP site refers to the nucleotide at position 24 from 5′ terminal of SEQ ID NO:30.

49. A method for identifying or assisting in identifying the kernel length of wheat grains, comprising the steps of:
detecting whether the genotype based on a 2144 SNP site in the genomic DNA of the wheat to be tested is AA genotype, AT genotype or TT genotype;
wherein
if the genotype is AA genotype, the wheat to be tested is selected as a candidate for wheat of long kernel length;
if the genotype is TT genotype, the wheat to be tested is selected as a candidate for wheat of short kernel length;
the wheat of long kernel length refers to wheat whose grains have a kernel length ≥0.65 mm;
the wheat of short kernel length refers to wheat whose grains have a kernel length <0.65 mm;
the 2144 SNP site refers to the nucleotide at position 24 from 5′ terminal of SEQ ID NO:30.

50. A primer set I, selected from the group consisting of 2144F1, 2144F2 and 2144C;
wherein primer 2144F1 comprises:
(b1) a single-stranded DNA molecule comprising SEQ ID NO:27; or
(b2) a DNA molecule obtained by subjecting SEQ ID NO: 27 to substitution or deletion or addition of one or several nucleotides and having the same function as SEQ ID NO:21;
wherein primer 2144F2 comprises:
(b3) a single-stranded DNA molecule as shown by SEQ ID NO:28; or
(b4) a DNA molecule obtained by subjecting SEQ ID NO: 28 to substitution or deletion or addition of one or several nucleotides and having the same function as SEQ ID NO:22;
wherein primer 2144C comprises:
(b5) a single-stranded DNA molecule comprising SEQ ID NO:29; or
(b6) a DNA molecule obtained by subjecting SEQ ID NO:29 to substitution and/or deletion and/or addition of one or several nucleotides and having the same function as SEQ ID NO:29.

51. A method for obtaining a wheat plant comprising the step of:
selecting a wheat plant with haplotype Hap I, wherein the wheat plan comprises:
(1) increased thousand-kernel weight in grains;
(2) increased kernel weight in grains;
(3) increased size in grains;
(4) increased length in grains;
(5) increased width in grains;
(6) increased thickness in grains;
(7) increased tiller length in plants;
(8) increased spike length in plants;
(9) increased grain number in plants; and
(10) increased grain yield in plants.
